(19) United States
(12) Patent Application Publication
(10) Pub. No.: US 2024/0268966 A1
(43) Pub. Date: Aug. 15, 2024

(54) APPARATUS, SYSTEM, AND METHOD FOR LAPIDUS CORRECTION

(71) Applicant: Treace Medical Concepts, Inc., Ponte Vedra, FL (US)

(72) Inventors: James Q. Spitler, Winter Garden, FL (US); Adam D. Perler, St. Petersburg, FL (US)

(21) Appl. No.: 18/439,454

(22) Filed: Feb. 12, 2024

Related U.S. Application Data
(60) Provisional application No. 63/484,492, filed on Feb. 11, 2023.

(51) Int. Cl. A61F 2/42; A61B 17/17; A61F 2/30; A61F 2/46

(52) U.S. Cl. CPC A61F 2/4225; A61B 17/1775; A61F 2/30942; A61F 2/4606; A61F 2002/30948; A61F 2002/30952; A61F 2002/4238; A61F 2002/4687

(57) ABSTRACT

An apparatus, system, and method are disclosed for remediating a condition present in a patient. In some implementations, the device may include a body. In addition, the device may include a bone engagement member on a bone-facing side of the body, the bone engagement member is configured to engage a first bone and a second bone such that when engaged by the device the first bone and a second bones are in a predetermined position. The device may include a reference feature guide configured with the body to guide formation of a reference feature for one of the first bone and the second bone.

### RELATED APPLICATIONS

This application claims the benefit of U.S. Provisional Application No. 63/484,492, filed Feb. 11, 2023, which is hereby incorporated by reference in its entirety.

### TECHNICAL FIELD

The present disclosure relates to surgical devices, systems, instruments, and methods. More specifically, the present disclosure relates to patient-specific guides, implants, instruments, and/or methods of designing and using the same.

### BACKGROUND

Various bone conditions may be corrected using surgical procedures, in which one or more tendons, ligaments, and/or bones may be cut, replaced, repositioned, reoriented, reattached, fixated and/or fused. These surgical procedures require the surgeon to properly locate, position, and/or orient one or more osteotomy cuts, fixation guides, fixators, bone tunnels, points of attachment for ends of grafts or soft tissue and the like. Determining and locating an optimal or desired location and trajectory for one or more steps of the surgical procedures can be challenging, given conventional techniques and instruments.

Hallux valgus or bunion conditions can be a source of discomfort, pain, and inconvenience for patients. Among a variety of different approaches for dealing with hallux valgus or bunion conditions, a Lapidus arthrodesis or simply Lapidus procedure is a common surgical procedure to address this condition.

Advancements in medical imaging, preoperative planning, modeling, and the like have led to improvements that help surgeons execute a Lapidus surgical procedure. What is needed is a solution that facilitates implementation of a preoperative plan or modeled correction and/or modeled procedure during the actual surgical procedure. The present disclosure provides such a solution.

### SUMMARY

The various apparatuses, devices, systems and methods of the present disclosure have been developed in response to the present state of the art, and in particular, in response to the problems and needs in the art that have not yet been fully solved by currently available apparatuses, devices, systems, and/or methods.

In one general aspect, the apparatus may include an apparatus for facilitating an osteotomy procedure. The apparatus may also include a body and a bone engagement member on a bone-facing side of the body, the bone engagement member configured to engage a first bone and a second bone. The apparatus may furthermore include a reference feature guide configured with the body to guide formation of a reference feature for one of the first bone and the second bone.

The described implementations may also include one or more of the following features. The apparatus where the bone engagement member is configured to fully engage with both the first bone and the second bone when both bones are in a predetermined position. The apparatus where the bone engagement member is configured to partially engage with one of the first bone and the second bone when one of the first bone and the second bone is in a predetermined position. The apparatus where the bone engagement member may include a bone engagement surface configured to engage with a cortical surface of at least one of the first bone and the second bone, the bone engagement surface having a contour at least partially determined based on a bone model of a patient's foot.

The apparatus where the bone engagement surface may include a first bone engagement surface configured to engage with the first bone and a second bone engagement surface configured to engage with the second bone. The apparatus where the first bone engagement surface engages with the first bone and the second bone engagement surface engages with the second bone when at least one of the first bone and the second bone is repositioned from an original position to a predetermined position.

The apparatus where the first bone engagement surface engages with the first bone and the second bone engagement surface engages with the second bone when the first bone maintains an original position and the second bone is repositioned from an original position to a predetermined position. The apparatus where the second bone engagement surface is configured to provide palpable feedback to an user when the second bone moves to a predetermined position and the second bone engagement surface engages with the second bone. The apparatus where the bone engagement surface may include a three-dimensional surface having an aspect that is patient-specific. The apparatus where the bone engagement member may include an opening in the body, the opening configured to accept at least a portion of one of the first bone and the second bone and at least partially determined based on a bone model. The apparatus where the reference feature guide may include at least one opening in the body that extends from a bone-facing side to a side opposite the bone-facing side.

The apparatus further may include: a handle configured with the body to extend the handle away from the body; and a landmark registration feature that extends from a bone-facing side of the body, the landmark registration feature configured to engage with a landmark of a patient. The apparatus further may include: a position indicator that indicates a position of the body in relation to the first bone and the second bone; and a window extending from a bone-facing side of the body to an opposite side of the body.

Some implementations herein relate to a system. For example, a system for an osteotomy procedure may include a positioner that provides a reference feature for an osteotomy procedure, the reference feature corresponding to a model reference of a model, the positioner having: a positioner body having a bone-facing side and a non-bone-facing side; a first bone engagement surface of the bone-facing side, the first bone engagement surface shaped to engage a surface of a first bone of a patient, the surface shaped based at least in part on a model of the first bone; a second bone engagement surface of the bone-facing side, the second bone engagement surface shaped to engage a surface of a second bone of a patient, the surface shaped based at least in part on a model of the second bone; a first reference feature guide formed in the positioner body and configured to guide formation of a first reference feature for the first bone; a second reference feature guide formed in the positioner body and configured to guide formation of a second reference feature for the second bone; and/or a handle coupled to the positioner body.

A system for an osteotomy procedure may also include a resection guide that couples to the reference feature, the resection guide having: a resection body having a bone-facing side and a non-bone-facing side; a first resection feature extending through the resection body from the non-bone-facing side of the resection body to the bone-facing side, the first resection feature configured to guide a cutting tool to form a first osteotomy in the first bone; a second resection feature extending through the resection body from the non-bone-facing side of the resection body to the bone-facing side, the second resection feature configured to guide a cutting tool to form a second osteotomy in the second bone; and a bone attachment feature configured to secure the resection guide to at least one of the first bone and the second bone.

The described implementations may also include one or more of the following features. A system where the first bone engagement surface is configured to engage the first bone when the first bone maintains an original position and the second bone engagement surface does not engage the second bone until the second bone is repositioned to a predetermined position. A system further may include: a compressor configured to compress a cut face of the first bone against a cut face of the second bone by engaging the first reference feature of the first bone and the second reference feature of the second bone; and an alignment guide configured to couple to the positioner and to secure a position indicator, the position indicator identifying a trajectory for one of the first bone and second bone after resecting the first bone and the second bone. A system where the first reference feature guide and second reference feature guide may include a set of holes that extend from the non-bone-facing side to the bone-facing side of the positioner body and where at least a first hole of the set of holes has a first trajectory that intersects a surface of the first bone at a perpendicular angle and at least a second hole of the set of holes has a second trajectory that intersects a surface of the second bone at a perpendicular angle. A system where the bone attachment feature may include a hole that extends from the non-bone-facing side to the bone-facing side of the resection body and has a third trajectory that intersects a surface of at least one of the first bone and the second bone at an oblique angle. A system where: the positioner is a patient-specific instrument made from a polymer; the first resection feature is configured to form a straight cut relative to a distal end of the first bone; the second resection feature is configured to form a straight cut relative to a proximal end of the second bone; and where the bone-facing side of the resection guide is planar. A system having a plurality of positioners each configured to engage the first bone in an original position and engage the second bone in a different predetermined position.

Some implementations herein relate to a method. For example, a method for remediating a bone condition present in a patient's foot, the method may include dissecting soft tissue around a tarsometatarsal (TMT) joint of a patient such that a metatarsal of the TMT joint can be repositioned.

The method may also include positioning a positioner such that a first bone engagement surface of the positioner engages a cuneiform of the TMT joint. A method may furthermore include moving the metatarsal such that a second bone engagement surface of the positioner engages the metatarsal. A may in addition include deploying a first set of guide pins through a first reference feature guide of the positioner and into the cuneiform such that the first set of guide pins forms a first reference feature.

A method may moreover include deploying a second set of guide pins through a second reference feature guide of the positioner and into the metatarsal such that the second set of guide pins forms a second reference feature. A method may also include positioning a non-patient-specific resection guide against the cuneiform and the metatarsal by way of the first reference feature and the second reference feature and securing the non-patient-specific resection guide to the cuneiform and to the metatarsal. A method may furthermore include resecting the cuneiform by way of a first resection feature of the non-patient-specific resection guide and the metatarsal by way of a second resection feature of the non-patient-specific resection guide. A method may in addition include deploying fixation to secure the cuneiform to the metatarsal.

### BRIEF DESCRIPTION OF THE DRAWINGS

The advantages, nature, and additional features of exemplary embodiments of the disclosure will become more fully apparent from the following description and appended claims, taken in conjunction with the accompanying drawings. Understanding that these drawings depict only exemplary embodiments and are, therefore, not to be considered limiting of the disclosure's scope, the exemplary embodiments of the disclosure will be described with additional specificity and detail through use of the accompanying drawings.

FIG. 1A is a flowchart diagram depicting a method for remediating a condition, according to one embodiment.

FIG. 1B is a flowchart diagram depicting a method for remediating a condition, according to one embodiment.

FIG. 2A is a dorsal perspective view of bones of a foot.

FIG. 2B is a lateral perspective view of bones of a foot.

FIG. 2C is a medial perspective view of bones of a foot.

FIG. 2D is a dorsal perspective view of bones of a foot.

FIG. 2E is a view of a foot illustrating common planes of reference for a human foot.

FIG. 3 is a flowchart diagram depicting a method for generating one or more patient-specific instruments configured to address a bone condition, according to one embodiment.

FIG. 4 illustrates an exemplary system configured to generate one or more patient-specific instruments configured to address a bone condition, according to one embodiment.

FIG. 5 illustrates an exemplary apparatus configured to determine a location and/or a trajectory for instrumentation, according to one embodiment.

FIG. 6 illustrates an exemplary provision module configured to provide a preliminary guide model, according to one embodiment.

FIG. 7 illustrates an exemplary design module configured to design a patient-specific guide model, according to one embodiment.

FIG. 8 illustrates an exemplary system configured to generate one or more patient-specific instruments configured to address a bone condition, according to one embodiment.

FIG. 9 illustrates a method for mapping from a model reference to a reference feature, according to one embodiment.

FIG. 10 illustrates a method for facilitating a surgical procedure, according to one embodiment.

FIG. 11 illustrates a method for implementing a surgical procedure, according to one embodiment.

FIG. 12 illustrates a method for implementing a surgical procedure, according to one embodiment.

FIG. 13 illustrates an exemplary system, according to one embodiment.

FIG. 14 illustrates an exemplary system, according to one embodiment.

FIGS. 15A-15G are top perspective, top, bottom, anterior, posterior, medial, and lateral views respectively, of an instrument according to one embodiment

FIGS. 16A-16G are top perspective, top, bottom, anterior, posterior, medial, and lateral views respectively, of an instrument according to one embodiment

FIGS. 17A-17F illustrate different views of stages of providing instrumentation, according to one embodiment.

FIGS. 18A-18F illustrate different views of stages of a surgical procedure, according to one embodiment.

FIG. 19 illustrates an exemplary system, according to one embodiment.

FIGS. 20A-20C illustrate different views of instrumentation of the system of FIG. 19, according to one embodiment.

FIG. 21 illustrates a method for a surgical procedure, according to one embodiment.

### DETAILED DESCRIPTION

Exemplary embodiments of the disclosure will be best understood by reference to the drawings, wherein like parts are designated by like numerals throughout. It will be readily understood that the components, as generally described and illustrated in the Figures herein, could be arranged and designed in a wide variety of different configurations. Thus, the following more detailed description of the embodiments of the apparatus, system, and method is not intended to limit the scope of the disclosure but is merely representative of exemplary embodiments.

The phrases “connected to,” “coupled to” and “in communication with” refer to any form of interaction between two or more entities, including mechanical, electrical, magnetic, electromagnetic, fluid, and thermal interaction. Two components may be functionally coupled to each other even though they are not in direct contact with each other. The term “abutting” refers to items that are in direct physical contact with each other, although the items may not necessarily be attached together. The phrase “fluid communication” refers to two features that are connected such that a fluid within one feature can pass into the other feature.

As used herein, “coupling”, “coupling member”, or “coupler” refers to a mechanical device, apparatus, member, component, system, assembly, or structure, that is organized, configured, designed, arranged, or engineered to connect, or facilitate the connection of, two or more parts, objects, or structures. In certain embodiments, a coupling can connect adjacent parts or objects at their ends. In certain embodiments, a coupling can be used to connect two shafts together at their ends for the purpose of transmitting power. In other embodiments, a coupling can be used to join two pieces of rotating equipment while permitting some degree of misalignment or end movement or both. In certain embodiments, couplings may not allow disconnection of the two parts, such as shafts during operation. (Search “coupling” on Wikipedia.com Jul. 26, 2021. CC-BY-SA 3.0 Modified. Accessed Jul. 27, 2021.) A coupler may be flexible, semiflexible, pliable, elastic, or rigid. A coupler may join two structures either directly by connecting directly to one structure and/or directly to the other or indirectly by connecting indirectly (by way of one or more intermediary structures) to one structure, to the other structure, or to both structures.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in drawings, the drawings are not necessarily drawn to scale unless specifically indicated.

Standard medical planes of reference and descriptive terminology are employed in this disclosure. While these terms are commonly used to refer to the human body, certain terms are applicable to physical objects in general. A standard system of three mutually perpendicular reference planes is employed. A sagittal plane divides a body into right and left portions. A coronal plane divides a body into anterior and posterior portions. A transverse plane divides a body into superior and inferior portions. A mid-sagittal, mid-coronal, or mid-transverse plane divides a body into equal portions, which may be bilaterally symmetric. The intersection of the sagittal and coronal planes defines a superior-inferior or cephalad-caudal axis. The intersection of the sagittal and transverse planes defines an anterior-posterior axis. The intersection of the coronal and transverse planes defines a medial-lateral axis. The superior-inferior or cephalad-caudal axis, the anterior-posterior axis, and the medial-lateral axis are mutually perpendicular.

Anterior means toward the front of a body. Posterior means toward the back of a body. Superior or cephalad means toward the head. Inferior or caudal means toward the feet or tail. Medial means toward the midline of a body, particularly toward a plane of bilateral symmetry of the body. Lateral means away from the midline of a body or away from a plane of bilateral symmetry of the body. Axial means toward a central axis of a body. Abaxial means away from a central axis of a body. Ipsilateral means on the same side of the body. Contralateral means on the opposite side of the body from the side which has a particular condition or structure. Proximal means toward the trunk of the body. Proximal may also mean toward a user, viewer, or operator. Distal means away from the trunk. Distal may also mean away from a user, viewer, or operator. Dorsal means toward the top of the foot or other body structure. Plantar means toward the sole of the foot or toward the bottom of the body structure.

Antegrade means forward moving from a proximal location/position to a distal location/position or moving in a forward direction. Retrograde means backward moving from a distal location/position to a proximal location/position or moving in a backwards direction. Sagittal refers to a midline of a patient's anatomy, which divides the body into left or right halves. The sagittal plane may be in the center of the body, splitting it into two halves. Prone means a body of a person lying face down. Supine means a body of a person lying face up.

“Patient-specific guide” refers to a guide designed, engineered, and/or fabricated for use with a specific patient. In one aspect, a patient-specific guide is unique to a patient and may include features unique to the patient such as a surface contour or other features.

“Patient-specific cutting guide” refers to a cutting guide designed, engineered, and/or fabricated for use with a specific patient. In one aspect, a patient-specific cutting guide is unique to a patient and may include features unique to the patient such as a surface contour or other features.

“Patient-specific resection guide” refers to a guide designed, engineered, and/or fabricated for use in resection for a specific patient. In one aspect, a patient-specific resection guide is unique to a patient and may include features unique to the patient such as a surface contour or other features.

“Patient specific instrument” (PSI) refers to a structure, device, guide, tool, instrument, apparatus, member, component, system, assembly, module, or subsystem that is adjusted, tailored, modified, organized, configured, designed, arranged, engineered, and/or fabricated to specifically address the anatomy, physiology, condition, abnormalities, needs, or desires of a particular patient. In certain aspects, one patient. In one aspect, a patient specific instrument is unique to a single patient and may include features unique to the patient such as a surface contour, component position, component orientation, and/or other features. In other aspects, one patient specific instrument may be useable with a number of patients having a particular class of characteristics.

“Instrument” refers to any apparatus, device, of object that can be used by a user. An instrument may be used for a specific or a generic purpose. An instrument may also be referred to as instrumentation. Instrumentation may refer to a single instrument and/or a plurality of instruments. An instrument may be specifically designed, constructed or fabricated for use by a specific user and/or for a single use. A patient specific instrument is one example of an instrument.

“Positioner” or “positioning guide” refers to any structure, apparatus, surface, device, system, feature, or aspect configured to position, move, translate, manipulate, or arrange one object in relation to another. In certain embodiments, a positioner can be used for one step in surgical procedure to position, arrange, orient, and/or reduce one bone or bone fragment relative to another. In such embodiments, the positioner may be referred to as a bone positioner. In certain embodiments, the term positioner or positioning guide may be preceded by an adjective that identifies the structure, implement, component, or instrument that may be used with, positioned by, and/or guided by with the positioner. For example, a “pin positioner” may be configured to accept a pin or wire such as a K-wire and serve to position or place the pin relative to another structure such as a bone.

“Compressor” refers to any apparatus, device, or system that can function as an active compression instrument. A compressor functions to bring two objects closer to or in contact with each other.

“Post” refers to any apparatus, structure, device, system, and/or component that extends from another structure. In certain embodiments, a post can be cylindrical.

“Cut surface” or “cut face” refers to a surface of an object that is created or formed by the removal of one or more parts of the object that includes the original surface. Cut surfaces or cut faces can be created using a variety of methods, tools, or apparatuses and may be formed using a variety of removal actions, including, but not limited to, fenestrating, drilling, abrading, cutting, sawing, chiseling, digging, scrapping, and the like. Tools and/or methods used for forming a cut surface or cut face can include manual, mechanical, motorized, hydraulic, automated, robotic, and the like. In certain embodiments, the cut surface(s) are planar.

“Reference” refers to any apparatus, structure, device, system, component, marking, and/or indicator organized, configured, designed, engineered, and/or arranged to serve as a source of information or a point of comparison used to support or establish knowledge, truth, or quality. (© ChatGPT January 9 Version, Modified, accessed chat.openai.com/chat Jan. 28, 2023). In certain embodiments, a reference can serve as a starting point or initial position for one or more steps in a surgical procedure. In certain embodiments, “reference” can be combined with an adjective describing the reference. For example, a “model reference” is a reference within a model such as a computer model. A model reference refers to any feature, aspect, and/or component within a model. Examples of a model reference include, but are not limited to, a point, a plane, a line, a plurality of points, a surface, an anatomical structure, a shape, or the like. An “anatomical reference” is a reference within, on, near, or otherwise associated with an anatomical structure such as a bone. A reference (e.g., model, actual, virtual, and/or real) may also be referred to as a reference feature.

“Reference feature” refers to a feature configured for use as a point, plane, axis, or line of reference (aka a reference). A reference can be used to position, measure, orient, fixation, couple, engage, and/or align one object or structure with another object or structure. In certain embodiments, a reference or reference feature can serve as a baseline, a ground truth, a waypoint, a control point, a landmark, and/or the like. A reference feature can facilitate moving from one coordinate system to a position or orientation on or in an actual object, structure, device, apparatus, anatomical structure, or the like. Advantageously, a reference feature can coordinate objects, models, or structures in a digital or virtual model or representation with corresponding objects or structures (e.g., anatomical structures) of actual physical objects or structures. Said another way, a reference feature can serve to map from a virtual or modeled object to an actual or physical object.

“Reference feature guide” refers to a guide that serves to aid in forming and/or deploying one or more reference features. Examples of reference feature guides include but are not limited to a hole, a round hole, a channel, a slot, a plurality of holes, a fence, a backstop, a guard, a fastener, a pilot hole, a blind hole, a chute, a ramp, or the like.

“Anatomical structure” refers to any part or portion of a part of a body of a person, animal, or other patient. Examples of anatomical structures, include but are not limited to, a bone, bones, soft tissue, a joint, joints, skin, hard tissue, teeth, mouth, eyes, hair, nails, fingers, toes, legs, arms, torso, vertebrae, ligaments, tendons, organs, or the like.

“Anatomical reference” or “anatomical landmark” refers to any reference or landmark that is, or is on, or is in, or is otherwise associated, with an anatomical structure. Examples of anatomical structures, include but are not limited to, a bone, bones, soft tissue, a joint, joints, skin, hard tissue, teeth, mouth, eyes, hair, nails, fingers, toes, legs, arms, torso, vertebrae, ligaments, tendons, organs, a hole, a post, a plurality of holes, a plurality of posts, or the like.

“Deformity” refers to an abnormality or deviation from the normal shape, structure, orientation, trajectory, or function of a body part. This can be due to congenital conditions, injuries, diseases, or other factors that alter the normal development or functioning of a part of the body. (© ChatGPT January 30 Version, accessed chat.openai.com/chat Feb. 7, 2023).

“Configuration” refers to an arrangement, setup, or values of one or more parts, features, settings, components, aspects, structures, or the like as a module, component, apparatus, device, system, framework, platform, dashboard, assembly, or the like. Examples of configurations can include how dials are setup on a dashboard, levers are set on a control board, switches are set within a controller, bones are arranged within a hand, foot, or limb, or the like.

“Interconnect” refers to a structure configured to join at least two other structures. In one embodiment, the interconnect may be a mechanical structure that may physically connect one structure to another structure. In other embodiments, an interconnect may be embodied as a fastener that enables permanent or temporary joining of one structure to another structure. In still other embodiments, an interconnect may be embodied as a joint or hinge configured to enable one or both structured joined by the interconnect to move relative to each other while remaining joined. In one embodiment, the interconnect may be configured to convey fluid and/or an electric signal between the at least two other structures. For example, the interconnect may comprise a channel or tube configured to convey air between a first opening and a second opening in the channel or tube. Examples of an interconnect include, but are not limited to, a pipe, a tunnel, a chamber, a channel, or the like. Other examples of an interconnect include, but are not limited to, solid material that can be additively manufactured between two structures, a snap, a hook and loop system, a spring, a tether, or the like.

As used herein, a “handle” refers to a structure used to hold, control, or manipulate a device, apparatus, component, tool, or the like. A “handle” may be designed to be grasped and/or held using one or two hands of a user.

As used herein, an “indicator” refers to an apparatus, device, component, system, assembly, mechanism, hardware, software, firmware, circuit, module, set of data, text, number, code, symbol, a mark, or logic structured, organized, configured, programmed, designed, arranged, or engineered to convey information or indicate a state, condition, mode, context, location, or position to another apparatus, device, component, system, assembly, mechanism, hardware, software, firmware, circuit, module, and/or a user of an apparatus, device, component, system, assembly, mechanism, hardware, software, firmware, circuit, module that includes, or is associated with the indicator. The indicator can include one or more of an audible signal, a token, a presence of a signal, an absence of a signal, a tactile signal, a visual signal or indication, a visual marker, a visual icon, a visual symbol, a visual code, a visual mark, and/or the like. In certain embodiments, “indicator” can be used with an adjective describing the indicator. For example, a “mode indicator” is an indicator that identifies or indicates a mode.

As used herein, a “marking” or “marker” refers to a symbol, letter, lettering, word, phrase, icon, design, color, diagram, indicator, figure, structure, device, apparatus, surface, component, system, or combination of these designed, intended, structured, organized, configured, programmed, arranged, or engineered to communication information and/or a message to a user receiving, viewing, or encountering the marking. The marking or “marker” can include one or more of a tactile signal, a visual signal or indication, an audible signal, and the like. In one embodiment, a marking may comprise a number or set letters, symbols, or words positioned on a surface, structure, color, color scheme, or device to convey a desired message or set of information.

As used herein, a “graft,” “tissue graft,” and/or “bone graft” refers to a surgical procedure to move tissue (hard and/or soft tissue) from one site to another on the body, or from another creature, without bringing its own blood supply with the tissue. Instead, a new blood supply grows in after the tissue is placed. A similar technique where tissue is transferred with the blood supply intact is called a flap. (Search ‘Graft (surgery)’ on Wikipedia.com Apr. 21, 2021. Modified. Accessed Aug. 30, 2021.) “Graft” may also be used to refer to the tissue and/or synthetic composition used for a graft surgical procedure. Bone grafting is a surgical procedure that replaces missing bone in order to repair bone fractures. Bone generally has the ability to regenerate completely but may require a small fracture space and/or a scaffold to do so. Bone grafts may be autologous (bone harvested from the patient's own body, often from the iliac crest), allograft (cadaveric bone usually obtained from a bone bank), or synthetic (often made of hydroxyapatite (HA) or other naturally occurring and biocompatible substances) with similar mechanical properties to bone. Generally, bone grafts are expected to be reabsorbed and replaced as natural bone heals over a few months' time. (Search ‘Bone Grafting’ on Wikipedia.com Apr. 21, 2021. Modified. Accessed Aug. 30, 2021.) Certain grafts may include a combination of autograft, isograft, allograft, xenograft, and/or synthetic materials in a single bone graft composition. An example of such a compositions, include but is not limited to, Demineralized bone matrix (DBM). Bone graft compositions may include bone morphogenetic proteins (BMPs).

As used herein, a “condition” refers to a state of something with regard to its appearance, quality, or working order. In certain aspects, a condition may refer to a patient's state of health or physical fitness or the state of health or physical fitness of an organ or anatomical part of a patient. In certain embodiments, a condition may refer to an illness, defect, disease, or deformity of a patient or of an organ or anatomical part of a patient. (Search “condition” on wordhippo.com. WordHippo, 2021. Web. Accessed 8 Dec. 2021. Modified.)

“Bone condition” refers to any of a variety of conditions of bones of a patient. Generally, a bone condition refers to an orientation, position, and/or alignment of one or more bones of the patient relative to other anatomical structures of the body of the patient. Bone conditions may be caused by or result from deformities, misalignment, malrotation, fractures, joint failure, and/or the like. A bone condition includes, but is not limited to, any angular deformities of one or more bone segments in either the lower or upper extremities (for example, tibial deformities, calcaneal deformities, femoral deformities, and radial deformities). Alternatively, or in addition, “bone condition” can refer to the structural makeup and configuration of one or more bones of a patient. Thus bone condition may refer to a state or condition of regions, a thickness of a cortex, bone density, a thickness and/or porosity of internal regions (e.g. whether it is calcaneus or solid) of the bone or parts of the bone such as a head, a base, a shaft, a protuberance, a process, a lamina, a foramen, and the like of a bone, along the metaphyseal region, epiphysis region, and/or a diaphyseal region.

“Data” refers to a set of information organized in a way that facilitates communication of the information to a receiver. The receiver may be a person or animal or an electronic component, circuit, assembly, or the like. Data can be represented as signal or values represented in any numbering and/or alphabet system. Data can be stored in one representation in an analog or digital format and conveyed to a receiver in another format suitable for the receiver to interpret and understand the data. Data can include both data that stores specific information as well as metadata which is data that describes the data that stores the specific information. Data can be organized in a structured or unstructured format. “Structured data” refers to data within a data structure that is organized according to a predefined format, protocol, or configuration such that the structure may be used to facilitate working with the data. Examples of structured data include, but are not limited to, files, databases, database records, database tables, database schemas, serialized objects, directories, and the like. “Unstructured data” refers to data stored without a particular organization, predefined format, protocol, or configuration. Examples of unstructured data include, but are not limited to, content of a text message, content of an email message, text content of a file, content of a document, and the like. Often the term “data” will be used in connection with one or more adjectives that identify a type or purpose for the data, examples include “user data”, “input data”, “output data”, “sensor data”, “patient data”, “system data”, “map data”, and the like. “Sensor data” refers to any data or information registered by one or more sensors. Examples of sensor data include an amount of current passing through the sensor, an amount of voltage across the sensor, an amount of electrical resistance through the sensor, an amount of strain experienced by the sensor, an acceleration vector, a deceleration vector, an orientation, an orientation angle, a direction, and the like.

As used herein, “implant” refers to a medical device manufactured to replace a missing biological structure, support a damaged biological structure, or enhance an existing biological structure. Often medical implants are man-made devices, but implants can also be natural occurring structures. The surface of implants that contact the body may be made of, or include a biomedical material such as titanium, cobalt chrome, stainless steel, carbon fiber, another metallic alloy, silicone, polymer, Synthetic polyvinyl alcohol (PVA) hydrogels, biomaterials, biocompatible polymers such as PolyEther Ether Ketone (PEEK) or a polylactide polymer (e.g. PLLA) and/or others, or apatite, or any combination of these depending on what is functional and/or economical. Implants can have a variety of configurations and can be wholly, partially, and/or include a number of components that are flexible, semiflexible, pliable, elastic, supple, semi-rigid, or rigid. In some cases, implants contain electronics, e.g. artificial pacemaker and cochlear implants. Some implants are bioactive, such as subcutaneous drug delivery devices in the form of implantable pills or drug-eluting stents. Orthopedic implants may be used to alleviate issues with bones and/or joints of a patient's body. Orthopedic implants can be used to treat bone fractures, osteoarthritis, scoliosis, spinal stenosis, discomfort, and pain. Examples of orthopedic implants include, but are not limited to, a wide variety of pins, rods, screws, anchors, spacers, sutures, all-suture implants, ball all-suture implants, self-locking suture implants, cross-threaded suture implants, plates used to anchor fractured bones while the bones heal or fuse together, and the like. (Search “implant (medicine)” on Wikipedia.com May 26, 2021. CC-BY-SA 3.0 Modified. Accessed Jun. 30, 2021.)

As used herein, a “body” refers to a main or central part of a structure. The body may serve as a structural component to connect, interconnect, surround, enclose, and/or protect one or more other structural components. A body may be made from a variety of materials including, but not limited to, metal, plastic, ceramic, wood, fiberglass, acrylic, carbon, biocompatible materials, biodegradable materials or the like. A body may be formed of any biocompatible materials, including but not limited to biocompatible metals such as Titanium, Titanium alloys, stainless steel alloys, cobalt-chromium steel alloys, nickel-titanium alloys, shape memory alloys such as Nitinol, biocompatible ceramics, and biocompatible polymers such as Polyether ether ketone (PEEK), a polylactide polymer (e.g. PLLA), nylon 12, and/or others. In one embodiment, a body may include a housing or frame, or framework for a larger system, component, structure, or device. A body may include a modifier that identifies a particular function, location, orientation, operation, and/or a particular structure relating to the body. Examples of such modifiers applied to a body, include, but are not limited to, “inferior body,” “superior body,” “lateral body,” “medial body,” and the like.

As used herein, “side” refers to a structure or part of a structure including, but not limited to: one of a longer bounding surfaces or lines of an object especially contrasted with the ends, a line or surface forming a border or face of an object, either surface of a thin object, a bounding line or structure of a geometric figure or shape, and the like. (search “side” on Merriam-Webster.com. Merriam-Webster, 2021. Web. 3 Aug. 2021. Modified.) A side can also refer to a geometric edge of a polygon (two-dimensional shape) and/or a face or surface of a polyhedron (three-dimensional shape). (Search “side” on Wikipedia.com Jul. 21, 2021. CC-BY-SA 3.0 Modified. Accessed Aug. 3, 2021.) Side can also refer to a location on a structure. For example, a side can be a location on a structure at, or near, a furthest position away from a central axis of the structure. As used herein, the term “side” can include one or more modifiers that define and/or orient and/or distinguish the side of an object from others based on based on where and/or how the object is deployed within or in relation to a second object. For example, in the context of an implant for a patient, sides of the implant may be labeled based on where the sides are relative to the patient when the implant is deployed. As one example, an “anterior side” of an implant, instrument, anatomical structure, or other structure refers to a side that is anterior to other sides of the structure in relation to a patient when the structure is deployed in the patient. As another example, in the context of an instrument used with a patient, sides of the instrument may be labeled based on where the sides are when the instrument is being used for its purpose. As one example, a “front side” of an instrument refers to a side that is facing a user of the instrument when the instrument is in use.

As used herein, “bone engagement surface” refers to a surface of an object, instrument, or apparatus, such as an implant that is oriented toward or faces one or more bones of a patient. In one aspect, the bone engagement surface may abut, touch, or contact a surface of a bone. In another aspect, the bone engagement surface or parts of the bone engagement surface may be close to, but not abut, touch, or contact a surface of the bone. In certain aspects, the bone engagement surface can be configured to engage with a surface of one or more bones. Such a bone engagement surface may include projections and recesses that correspond to and match projections and recesses of the one or more bone surfaces.

“Bone engagement member” refers to an apparatus, instrument, structure, device, component, member, system, assembly or module structured, organized, configured, designed, arranged, or engineered to connect, join, link, contact, touch, abut, interface with, couple to, or engage with a bone, a bone part, bony topography (e.g., bone spurs and calcifications), anatomical bone feature, and/or a bone fragment. The connection, coupling, linkage, contact, or engagement may be a mechanical connection or interconnection. A bone engagement member may enable temporary engagement with a bone or bone fragment or permanent engagement with a bone or bone fragment. A bone engagement member may include a bone engagement surface, a bone engagement feature, a body section that supports the bone engagement surface, or the like. In certain embodiments, a bone engagement member may include a bone probe or a joint seeker. In one embodiment, a bone engagement member may include a landmark registration feature. Alternatively, or in addition, a bone engagement member can include a bone attachment feature configured to engage with bone and/or to cooperate with a fastener to engage with bone. A patient-specific bone engagement member is a bone engagement member that includes one or more aspects that are patient-specific. The patient-specific aspects can include, but are not limited to, a surface contour, a contour for a part of a surface, a position for a resection feature, a size, shape and/or configuration of a resection feature, a position, size, shape, and/or number of bone attachment features, or the like.

As used herein, “bone-facing side” refers to a side of an object, structure, instrument, or apparatus, such as an implant or instrument that is oriented toward or faces one or more bones of a patient when a device that includes the bone-facing side is in use. In one aspect, the bone-facing side may abut, touch, or contact a surface of a bone. In another aspect, the bone-facing side or parts of the bone-facing side may be close to, but not abut, touch, or contact a surface of the bone.

“Non-bone-facing side” refers to a side of an object, structure, instrument, or apparatus, such as an implant or instrument that is not oriented toward and/or does not face one or more bones of a patient during use of a device that includes the non-bone-facing side. In certain embodiments, a non-bone-facing side can be a side that is directly opposite a bone-facing side of the same device, object, structure, or apparatus.

“Cortical surface” refers to a surface of cortical bone. “Cortical bone” refers to a type of bone tissue. Cortical bone is a type of bone tissue typically found between an external surface of a bone and an interior area of the bone. Cortical bone is more dense and typically stronger structurally than other types of bone tissue.

“Predetermined Position” refers to a position that is decided, determined, finalized, and/or defined earlier in time. In certain embodiments, a predetermined position is a desired, designed, and/or engineered position of a first object in relation to a second object. Thus, a predetermined position is a planned position for the two objects in relation to each other. In certain embodiments, one or both of the two objects may be moved relative to each other to accomplish the predetermined position and the predetermined position may become the final position. In other embodiments, the two objects may be moved towards the predetermined position but may not reach the exact predetermined position due to some impediment and/or interference or a decision to change the predetermined position to a new position. In certain aspects, a predetermined position may be a position that is decided after a process of recommendation, review, and/or analysis, and final approval such that a position may not become a predetermined position until the process is completed. For example, in a medical patient-specific instrument or technique design process a position may not become the predetermined position until a surgeon or other doctor provides final approval for the position. In certain embodiments, a predetermined position may be indicated, designated, illustrated, defined, and/or explained in a preoperative plan.

As used herein, a “deploy” or “deployment” refers to an act, action, process, system, method, means, or apparatus for inserting an implant or prosthesis into a part, body part, and/or patient. “Deploy” or “deployment” can also refer to an act, action, process, system, method, means, or apparatus for placing something into therapeutic use. A device, system, component, medication, drug, compound, or nutrient may be deployed by a human operator, a mechanical device, an automated system, a computer system or program, a robotic system, or the like.

“Joint” or “Articulation” refers to the connection made between bones in a human or animal body which link the skeletal system to form a functional whole. Joints may be biomechanically classified as a simple joint, a compound joint, or a complex joint. Joints may be classified anatomically into groups such as joints of hand, elbow joints, wrist joints, axillary joints, sternoclavicular joints, vertebral articulations, temporomandibular joints, sacroiliac joints, hip joints, knee joints, articulations of foot, and the like. (Search “joint” on Wikipedia.com Dec. 19, 2021. CC-BY-SA 3.0 Modified. Accessed Jan. 20, 2022.)

“Contour” refers to an outline representing or bounding a shape or form of an object. Contour can also refer to an outside limit of an object, area, or surface of the object. (Search “contour” on wordhippo.com. WordHippo, 2023. Web. Modified. Accessed 13 Jun. 2023.)

“Topographical” refers to the physical distribution of parts, structures, or features on the surface of, or within, an organ or other anatomical structure, or organism. (Search “define topographical” on google.com. Oxford Languages, Copyright 2022. Oxford University Press. Web., Modified. Accessed 15 Feb. 2022.)

“Landmark registration feature” refers to a structure configured to engage, contact, or abut a feature, aspect, attribute, or characteristic of a first object to orient and/or position a second object that includes the landmark registration feature with respect to the first object. A variety of structures can serve as a landmark registration feature. For example, a surface, a probe, a finger, a wing, an arm, an opening, or the like can function as landmark registration features. A landmark registration feature can be of a variety of shapes and thus can include a protrusion, a projection, a tuberosity, a cavity, a void, a divot, a tab, an extension, a hook, a curve, or the like.

“Landmark” refers to a structure on, in, or around a structure that can be used to serve as a reference for positioning, orienting, translating, rotating, or otherwise manipulating a second object or structure. For example, a landmark may include a protrusion, a projection, a tuberosity, a cavity, a void, a divot, a tab, an extension, a hook, a curve, or the like. In the context of bones of a patient, a landmark can include any protuberance, eminence, bony topography, anatomical features, calcifications, void, divot, concave section, sesamoid, bone spur or other feature on, or extending from, a bone of a patient. A landmark refers to any structure of an anatomical structure that is referenced, contacted, engaged with and/or associated with a landmark registration feature. In certain embodiments, a landmark is unique to one patient.

“Bone attachment feature” refers to a structure, feature, component, aspect configured to securely connect, couple, attach, and/or engage a structure, component, object, or body with a bone and/or a bone fragment. Examples of a bone attachment feature, include, but are not limited to, a pin, K-wire, screw, or other fastener alone, or in combination with, a hole, passage, and/or opening.

“Position indicator” refers to any apparatus, structure, device, system, and/or component organized, configured, designed, engineered, and/or arranged to serve as an indicator of a position for one or more things, objects, structures, apparatuses, systems, features, aspects, attributes or the like. Examples of a position indicator include, but are not limited to, a crosshair, cross hairs, a pin, a wire, a fastener, a hole, an opening, a post, a prong, a probe, a needle, an arrow, a marking, or the like. In certain embodiments, an indicator may communicate a position of one structure or component or system in relation to another. A position indicator may indicate a position of one object relative to another, may indicate a relationship between two objects, may indicate a trajectory of one object relative to another, or the like.

As used herein, a “stop” refers to an apparatus, instrument, structure, member, device, component, system, or assembly structured, organized, configured, designed, arranged, or engineered to prevent, limit, impede, stop, or restrict motion or movement and/or operation of the another object, member, structure, component, part, apparatus, system, or assembly.

“Window” refers to an opening and/or a plurality of openings in a body, side, wall, side door, roof, vehicle, system, component, or other structure that allows the passage of electromagnetic radiation including radio ways, x-rays, visible light, light, and the like. A window may also permit passage of sound, gases, fluids, liquids, or other elements. (Search “window” on Wikipedia.com Aug. 31, 2022. Modified. Accessed Sep. 21, 2022.). A window can be opaque, semi-opaque, translucent, radiolucent, or transparent. A window can include a single opening having a single geometric shape or a plurality of openings each of a single geometric shape or combination of a variety of geometric shapes. In certain embodiments, a window may be referred to as a radiolucent window. A radiolucent window may permit passage of some or all radio ways through the window.

“Radiolucent window” refers to a window that permits the passage of radiant energy and electromagnetic radiant energy, in particular, such as x-rays used in an x-ray machine and/or in a fluoroscopy imaging device.

As used herein, a “fastener”, “fixation device”, or “fastener system” refers to any structure configured, designed, or engineered to join two structures. Fasteners may be made of a variety of materials including metal, plastic, composite materials, metal alloys, plastic composites, and the like. Examples of fasteners include, but are not limited to screws, rivets, bolts, nails, snaps, hook and loop, set screws, bone screws, nuts, posts, pins, thumb screws, and the like. Other examples of fasteners include, but are not limited to wires, Kirschner wires (K-wire), anchors, bone anchors, plates, bone plates, intramedullary nails or rods or pins, implants, sutures, soft sutures, soft anchors, tethers, interbody cages, fusion cages, and the like.

In certain embodiments, the term fastener may refer to a fastener system that includes two or more structures configured to combine to serve as a fastener. An example of a fastener system is a rod or shaft having external threads and an opening or bore within another structure having corresponding internal threads configured to engage the external threads of the rod or shaft.

In certain embodiments, the term fastener may be used with an adjective that identifies an object or structure that the fastener may be particularly configured, designed, or engineered to engage, connect to, join, contact, or couple together with one or more other structures of the same or different types. For example, a “bone fastener” may refer to an apparatus for joining or connecting one or more bones, one or more bone portions, soft tissue and a bone or bone portion, hard tissue and a bone or bone portion, an apparatus and a bone or portion of bone, or the like.

In certain embodiments, a fastener may be a temporary fastener. A temporary fastener is configured to engage and serve a fastening function for a relatively short period of time. Typically, a temporary fastener is configured to be used until another procedure or operation is completed and/or until a particular event. In certain embodiments, a user may remove or disengage a temporary fastener. Alternatively, or in addition, another structure, event, or machine may cause the temporary fastener to become disengaged.

As used herein, a “fixator” refers to an apparatus, instrument, structure, device, component, member, system, assembly, or module structured, organized, configured, designed, arranged, or engineered to connect two bones or bone fragments or a single bone or bone fragment and another fixator to position and retain the bone or bone fragments in a desired position and/or orientation. Examples of fixators include both those for external fixation as well as those for internal fixation and include, but are not limited to pins, wires, Kirschner wires, screws, anchors, bone anchors, plates, bone plates, intramedullary nails or rods or pins, implants, interbody cages, fusion cages, and the like.

As used herein, a “fixation” or “fixation device” refers to an apparatus, instrument, structure, device, component, member, system, assembly, step, process, or module structured, organized, configured, designed, arranged, or engineered to connect two structures either permanently or temporarily. The two structures may be one or the other or both of manmade and/or biological tissues, hard tissues such as bones, teeth or the like, soft tissues such as ligament, cartilage, tendon, or the like. In certain embodiments, fixation is used as an adjective to describe a device or component or step in securing two structures such that the structures remain connected to each other in a desired position and/or orientation. Fixation devices can also serve to maintain a desired level of tension, compression, or redistribute load and stresses experienced by the two structures and can serve to reduce relative motion of one part relative to others. Examples of fixation devices are many and include both those for external fixation as well as those for internal fixation and include, but are not limited to pins, wires, Kirschner wires (K-wires), screws, anchors, bone anchors, plates, bone plates, intramedullary nails or rods or pins, implants, interbody cages, fusion cages, and the like.

As used herein, an “anchor” refers to an apparatus, instrument, structure, member, part, device, component, system, or assembly structured, organized, configured, designed, arranged, or engineered to secure, retain, stop, and/or hold, an object to or at a fixed point, position, or location. An anchor may be coupled and/or connected to a flexible member such as a tether, chain, rope, wire, thread, suture, suture tape, or other like object. Alternatively, or in addition, an anchor may also be coupled, connected, and/or joined to a rigid object or structure. In certain embodiments, an anchor can be a fixation device. Said another way, a fixation device can function as an anchor. For example, an anchor pin is a pin, fastener, or K-wire that cooperates with a rigid structure to provide an anchor.

“Connector” refers to any structure configured, engineered, designed, adapted, and/or arranged to connect one structure, component, element, or apparatus to another structure, component, element, or apparatus. A connector can be rigid, pliable, elastic, flexible, and/or semiflexible. Examples of a connector include but are not limited to any fastener.

As used herein, a “sleeve” refers to structure that is narrow and longer longitudinally than the structure is wide. In certain embodiments, a sleeve serves to surround, enclose, wrap, and/or contain something else. In certain embodiments, a sleeve may surround, enclose, wrap, and/or contain a passage or void. (Search “sleeve” on wordhippo.com. WordHippo, 2021. Web. Accessed 15 Nov. 2021. Modified.) In certain embodiments, the term sleeve may be preceded by an adjective that identifies the structure, implement, component or instrument that may be used with, inserted into or associated with the sleeve. For example, a “pin sleeve” may be configured to accept a pin or wire such as a K-wire, a “drive sleeve” may be configured to accept a drill or drill bit, a “fixation member sleeve” may be configured to accept a fastener or fixation member.

As used herein, a “long bone” refers to a bone of a patient having a length greater than a width of the bone. Long bone is one of five types of bones: long, short, flat, irregular and sesamoid. Long bones, especially the femur and tibia, can be subjected to most of the load during daily activities. Long bones grow primarily by elongation of the diaphysis, with an epiphysis at each end of the growing bone. The ends of epiphyses are covered with hyaline cartilage (“articular cartilage”). The longitudinal growth of long bones is a result of endochondral ossification at the epiphyseal plate. The long bone category type includes the femur, tibia, and fibula of the legs; the humerus, radius, and ulna of the arms; metacarpals and metatarsals of the hands and feet, the phalanges of the fingers and toes, and the clavicles or collar bones in humans or other patients. The outside of the long bone consists of a layer of connective tissue called the periosteum. Additionally, the outer shell of the long bone is compact bone, then a deeper layer of cancellous bone (spongy bone) which includes a medullary cavity that includes bone marrow. (Search “long bone” on Wikipedia.com May 14, 2021. CC-BY-SA 3.0 Modified. Accessed Jul. 26, 2021.)

“Talar dome” refers to part of a talus bone. Specifically, the talar dome refers to the superior convex surface and/or area of the talus. The talar dome may also be referred to as a trochlea of the talus. The talar dome is part of the talus body.

“Bone fragment” or “fragment” refers to a part of a bone that is normally part of another bone of a patient. A bone fragment may be separate from another bone of a patient due to a deformity or trauma. In one aspect, the bone the bone fragment is normally connected or joined with is referred to as a parent bone.

As used herein, “manufacturing tool” or “fabrication tool” refers to a manufacturing or fabrication process, tool, system, or apparatus which creates an object, device, apparatus, feature, or component using one or more source materials. A manufacturing tool or fabrication tool can use a variety of manufacturing processes, including but not limited to additive manufacturing, subtractive manufacturing, forging, casting, and the like. The manufacturing tool can use a variety of materials including polymers, thermoplastics, metals, biocompatible materials, biodegradable materials, ceramics, biochemicals, and the like. A manufacturing tool may be operated manually by an operator, automatically using a computer numerical controller (CNC), or a combination of these techniques.

As used herein, “osteotomy procedure” or “surgical osteotomy” refers to a surgical operation in which one or more bones are cut to shorten or lengthen them or to change their alignment. The procedure can include removing one or more portions of bone and/or adding one or more portions of bone or bone substitutes. (Search “osteotomy” on Wikipedia.com Feb. 3, 22, 2021. CC-BY-SA 3.0 Modified. Accessed Feb. 15, 2022.)

As used herein, “patient-specific osteotomy procedure” refers to an osteotomy procedure that has been adjusted, tailored, modified, or configured to specifically address the anatomy, physiology, condition, abnormalities, needs, or desires of a particular patient. In certain aspects, one patient-specific osteotomy procedure may be useable in connection with only one patient. In other aspects, one patient-specific osteotomy procedure may be useable with a number of patients having a particular class of characteristics. In certain aspects, a patient-specific osteotomy procedure may refer to a non-patient-specific osteotomy procedure that includes one or more patient-specific implants and/or instrumentation. In another aspects, a patient-specific osteotomy procedure may refer to a patient-specific osteotomy procedure that includes one or more patient-specific implants, patient-specific surgical steps, and/or patient-specific instrumentation.

“Register” or “Registration” refers to an act of aligning, mating, contacting, engaging, or coupling one or more parts and/or surfaces of one object in relation to one or more parts and/or surfaces of another object. Often, the one or more parts and/or surfaces of one object include protrusions and/or depressions that are the inverse or mirror configuration of protrusions and/or depressions of one or more parts and/or surfaces of the other object.

“Remediation procedure” refers to any designed or performed for the purpose of remediating a condition of a patient and/or a condition of one or more parts of a body of a patient.

“Wedge osteotomy” refers to an osteotomy procedure in which one or more wedges are used as part of the procedure. Generally, wedge osteotomies can be of one of two types, open wedge and closing wedge. The type of osteotomy refers to how the procedure changes the relation between two parts of a bone involved in the osteotomy. In an open wedge osteotomy, a wedge of bone or graft or other material is inserted in between two parts of a bone. Consequently, a wedge shape is “opened” in the bone. In a close wedge osteotomy or closing wedge osteotomy a wedge of bone is removed from a bone. Consequently, a wedge shape formed in the bone is “closed.”

As used herein, an “opening” refers to a gap, a hole, an aperture, a port, a portal, a slit, a space or recess in a structure, a void in a structure, or the like. In certain embodiments, an opening can refer to a structure configured specifically for receiving something and/or for allowing access. In certain embodiments, an opening can pass through a structure. In such embodiments, the opening can be referred to as a window. In other embodiments, an opening can exist within a structure but not pass through the structure. In other embodiments, an opening can initiate on a surface or at an edge or at a side of a structure and extend into the structure for a distance, but not pass through or extend to another side or edge of the structure. In other embodiments, an opening can initiate on a surface or at an edge or at a side of a structure and extend into the structure until the opening extends through or extends to another side or edge of the structure. An opening can be two-dimensional or three-dimensional and can have a variety of geometric shapes and/or cross-sectional shapes, including, but not limited to a rectangle, a square, or other polygon, as well as a circle, an ellipse, an ovoid, or other circular or semi-circular shape. As used herein, the term “opening” can include one or more modifiers that define specific types of “openings” based on the purpose, function, operation, position, or location of the “opening.” As one example, a “fastener opening” refers to an “opening” adapted, configured, designed, or engineered to accept or accommodate a “fastener.”

“Hole” refers to a gap, an opening, an aperture, a port, a portal, a space or recess in a structure, a void in a structure, or the like. In certain embodiments, a hole can refer to a structure configured specifically for receiving something and/or for allowing access. In certain embodiments, a hole can pass through a structure. In other embodiments, an opening can exist within a structure but not pass through the structure. A hole can be two-dimensional or three-dimensional and can have a variety of geometric shapes and/or cross-sectional shapes, including, but not limited to a rectangle, a square, or other polygon, as well as a circle, an ellipse, an ovoid, or other circular or semi-circular shape. As used herein, the term “hole” can include one or more modifiers that define specific types of “holes” based on the purpose, function, operation, position, or location of the “hole.” As one example, a “fastener hole” refers to an “hole” adapted, configured, designed, or engineered to accept or accommodate a “fastener.” A “blind hole” is a hole with an opening on one side that does not extend all the way through a structure. In certain embodiments, a hole, including a blind hole, has a circular longitudinal cross-section. Alternatively, or in addition, a hole can have a cross-section of a variety of geometric shapes include a circle, an oval, a square, a rectangle, a slot with rounded ends, a triangle, or the like.

As used herein, “anatomic data” refers to data identified, used, collected, gathered, and/or generated in connection with an anatomy of a human or animal. Examples of anatomic data may include location data for structures, both independent, and those connected to other structures within a coordinate system. Anatomic data may also include data that labels or identifies one or more anatomical structures. Anatomic data can include volumetric data, material composition data, and/or the like. Anatomic data can be generated based on medical imaging data or measurements using a variety of instruments including monitors and/or sensors. Anatomic data can be gathered, measured, or collected from anatomical models and/or can be used to generate, manipulate, or modify anatomical models.

A bone model or anatomic model of a patient's body or body part(s) may be generated by computing devices that analyze medical imaging images. Structures of a patient's body can be determined using a process called segmentation.

“Set” refers to a collection of objects. A set can have zero or more objects in the collection. Generally, a set includes one or more objects in the collection.

“Trajectory guide” or “trajectory indicator” or “targeting guide” refers to any structure, apparatus, surface, device, system, feature, or aspect configured to indicate, identify, guide, place, position, or otherwise assist in marking or deploying a fastener or other structure along a desired trajectory for one or more subsequent steps in a procedure.

“Trajectory” refers to a path a body travels or a path configured for a body to travel through space. (Search “trajectory” on wordhippo.com. WordHippo, 2023. Web. Modified. Accessed 13 Jun. 2023.)

As used herein, a “guard” refers to an apparatus, instrument, structure, member, device, component, system, or assembly structured, organized, configured, designed, arranged, or engineered to prevent, limit, impede, stop, or restrict motion, action, or movement and/or operation of the another object, member, structure, component, part, apparatus, system, or assembly beyond a certain parameter such as a boundary. Said another way, a “guard” refers to an apparatus, instrument, structure, member, device, component, system, or assembly structured, organized, configured, designed, arranged, or engineered to retain, maintain, hold, keep, or restrict motion, action, or movement and/or operation of the another object, member, structure, component, part, apparatus, system, or assembly within or at one or more parameters such as a boundary.

As used herein, “artificial intelligence” refers to intelligence demonstrated by machines, unlike the natural intelligence displayed by humans and animals, which involves consciousness and emotionality. The distinction between artificial intelligence and natural intelligence categories is often revealed by the acronym chosen. ‘Strong’ AI is usually labelled as artificial general intelligence (AGI) while attempts to emulate ‘natural’ intelligence have been called artificial biological intelligence (ABI). Leading AI textbooks define the field as the study of “intelligent agents”: any device that perceives its environment and takes actions that maximize its chance of achieving its goals. The term “artificial intelligence” can also be used to describe machines that mimic “cognitive” functions that humans associate with the human mind, such as “learning” and “problem solving”. (Search “artificial intelligence” on Wikipedia.com Jun. 25, 2021. CC-BY-SA 3.0 Modified. Accessed Jun. 25, 2021.)

As used herein, “segmentation” or “image segmentation” refers to the process of partitioning an image into different meaningful segments. These segments may correspond to different tissue classes, organs, pathologies, bones, or other biologically relevant structures. Medical image segmentation accommodates imaging ambiguities such as low contrast, noise, and other imaging ambiguities.

Certain computer vision techniques can be used or adapted for image segmentation. For example, the techniques and or algorithms for segmentation may include, but are not limited to: Atlas-Based Segmentation: For many applications, a clinical expert can manually label several images; segmenting unseen images is a matter of extrapolating from these manually labeled training images. Methods of this style are typically referred to as atlas-based segmentation methods. Parametric atlas methods typically combine these training images into a single atlas image, while nonparametric atlas methods typically use all of the training images separately. Atlas-based methods usually require the use of image registration in order to align the atlas image or images to a new, unseen image.

Image registration is a process of correctly aligning images; Shape-Based Segmentation: Many methods parametrize a template shape for a given structure, often relying on control points along the boundary. The entire shape is then deformed to match a new image. Two of the most common shape-based techniques are Active Shape Models and Active Appearance Models; Image-Based Segmentation: Some methods initiate a template and refine its shape according to the image data while minimizing integral error measures, like the Active contour model and its variations; Interactive Segmentation: Interactive methods are useful when clinicians can provide some information, such as a seed region or rough outline of the region to segment. An algorithm can then iteratively refine such a segmentation, with or without guidance from the clinician. Manual segmentation, using tools such as a paint brush to explicitly define the tissue class of each pixel, remains the gold standard for many imaging applications. Recently, principles from feedback control theory have been incorporated into segmentation, which give the user much greater flexibility and allow for the automatic correction of errors; Subjective surface Segmentation: This method is based on the idea of evolution of segmentation function which is governed by an advection-diffusion model. To segment an object, a segmentation seed is needed (that is the starting point that determines the approximate position of the object in the image). Consequently, an initial segmentation function is constructed. With the subjective surface method, the position of the seed is the main factor determining the form of this segmentation function; and Hybrid segmentation which is based on combination of methods. (Search “medical image computing” on Wikipedia.com Jun. 24, 2021. CC-BY-SA 3.0 Modified. Accessed Jun. 24, 2021.)

As used herein, “medical imaging” refers to a technique and process of imaging the interior or exterior of a body for clinical analysis and medical intervention, as well as visual representation of the function of some organs or tissues (physiology). Medical imaging seeks to reveal internal structures hidden by the skin and bones, as well as to diagnose and treat disease. Medical imaging may be used to establish a database of normal anatomy and physiology to make possible identification of abnormalities.

Medical imaging in its widest sense, is part of biological imaging and incorporates radiology, which uses the imaging technologies of X-ray radiography, magnetic resonance imaging, ultrasound, endoscopy, elastography, tactile imaging, thermography, medical photography, nuclear medicine functional imaging techniques as positron emission tomography (PET) and single-photon emission computed tomography (SPECT). Another form of X-ray radiography includes computerized tomography (CT) scans in which a computer may control the position of the X-ray sources and detectors. Magnetic Resonance Imaging (MRI) is another medical imaging technology. Fluoroscopy is an imaging technique that uses X-rays to obtain real-time moving images of the interior of an object. In its primary application of medical imaging, a fluoroscope allows a physician to see the internal structure and function of a patient, so that the pumping action of the heart or the motion of swallowing, for example, can be watched. This is useful for both diagnosis and therapy and occurs in general radiology, interventional radiology, and image-guided surgery. (Search “medical imaging” on Wikipedia.com Jul. 14, 2021. CC-BY-SA 3.0 Modified. Accessed Sep. 1, 2021.)

Data analyzed, generated, manipulated, interpolated, collected, stored, reviewed, and/or modified in connection with medical imaging or medical image processing can be referred to herein as medical imaging data or medical image data.

Measurement and recording techniques that are not primarily designed to produce images, such as electroencephalography (EEG), magnetoencephalography (MEG), electrocardiography (ECG), and others, represent other technologies that produce data susceptible to representation as a parameter graph vs. time or maps that contain data about the measurement locations. In certain embodiments bone imaging includes devices that scan and gather bone density anatomic data. These technologies may be considered forms of medical imaging in certain disciplines. (Search “medical imaging” on Wikipedia.com Jun. 16, 2021. CC-BY-SA 3.0 Modified. Accessed Jun. 23, 2021.)

As used herein, As used herein, “patient imaging data” refers to data identified, used, collected, gathered, and/or generated in connection with medical imaging for a particular patient. Patient imaging data is one type of medical imaging data. Patient imaging data can be shared between users, systems, patients, and professionals using a common data format referred to as Digital Imaging and Communications in Medicine (DICOM) data. DICOM data is a standard format for storing, viewing, retrieving, and sharing medical images.

As used herein, “medical image computing” or “medical image processing” refers to systems, software, hardware, components, and/or apparatus that involve and combine the fields of computer science, information engineering, electrical engineering, physics, mathematics and medicine. Medical image computing develops computational and mathematical methods for working with medical images and their use for biomedical research and clinical care. One goal for medical image computing is to extract clinically relevant information or knowledge from medical images. While closely related to the field of medical imaging, medical image computing focuses on the computational analysis of the images, not their acquisition. The methods can be grouped into several broad categories: image segmentation, image registration, image-based physiological modeling, and others. (Search “medical image computing” on Wikipedia.com Jun. 24, 2021. CC-BY-SA 3.0 Modified. Accessed Jun. 24, 2021.) Medical image computing may include one or more processors or controllers on one or more computing devices. Such processors or controllers may be referred to herein as medical image processors. Medical imaging and medical image computing together can provide systems and methods to image, quantify and fuse both structural and functional information about a patient in vivo. These two technologies include the transformation of computational models to represent specific subjects/patients, thus paving the way for personalized computational models. Individualization of generic computational models through imaging can be realized in three complementary directions: definition of the subject-specific computational domain (anatomy) and related subdomains (tissue types); definition of boundary and initial conditions from (dynamic and/or functional) imaging; and characterization of structural and functional tissue properties. Medical imaging and medical image computing enable the translation of models to the clinical setting with both diagnostic and therapeutic applications. (Id.) In certain embodiments, medical image computing can be used to generate a bone model, a patient-specific model, and/or a patent specific instrument from medical imaging and/or medical imaging data.

As used herein, “model” refers to an informative representation of an object, person or system. Representational models can be broadly divided into the concrete (e.g. physical form) and the abstract (e.g. behavioral patterns, especially as expressed in mathematical form). In abstract form, certain models may be based on data used in a computer system or software program to represent the model. Such models can be referred to as computer models. Computer models can be used to display the model, modify the model, print the model (either on a 2D medium or using a 3D printer or additive manufacturing technology). The printed physical form of the model can be referred to as a 3D model. Computer models can also be used in environments with models of other objects, people, or systems. Computer models can also be used to generate simulations, display in virtual environment systems, display in augmented reality systems, or the like. Computer models can be used in Computer Aided Design (CAD) and/or Computer Aided Manufacturing (CAM) systems. Certain models may be identified with an adjective that identifies the object, person, or system the model represents. For example, a “bone” model is a model of a bone, and a “heart” model is a model of a heart. (Search “model” on Wikipedia.com Jun. 13, 2021. CC-BY-SA 3.0 Modified. Accessed Jun. 23, 2021.)

A “bone model” or “anatomic model” refers to a model of a bone of a person. The bone model may model a single bone or a plurality of bones. The modeled bone and/or bones may be positioned in standard anatomical form and/or may be positioned relative to other bones (e.g., models of bones) of a person such that the positions of the bones in the bone model are the same or substantially the same as corresponding bones of a person, such as a patient.

“Position” refers to a place or location. (Search “position” on wordhippo.com. WordHippo, 2024. Web. Accessed 8 Jan. 2024.) A position may be defined in a virtual environment such as in a model or set of models defined by and presented by a computing device. In addition, a position may be a place or location in a tangible physical environment such as in a space, on land, within or on a system, assembly, component, a patient, or other structure.

“Original position” refers to a position before any actions are taken to change a position of a structure, object, device, apparatus, component, or system. An original position may be defined in a virtual environment such as in a model or set of models defined by and presented by a computing device. Alternatively, or in addition, an original position may be a position in, on, or part of a tangible physical object, such as bones of a foot in a patient. In certain embodiments, an original position is a deformed position. An original position can be contrasted with a predetermined position which may be a position planned to implement a correction, correct a structure's position from a deformed position to a corrected position.

A “deformed position” refers to an anatomical structure that is positioned to form, include, or is at least part of a deformity. A “corrected position” refers to an anatomical structure positioned to remediate, correct, eliminate, and/or overcome a deformity. A predetermined position can be a corrected position.

“Feedback” refers to a reactionary response to an action, a product, service, or task. (Search “feedback” on wordhippo.com. WordHippo, 2023. Web. Modified. Accessed 28 August 2023.)

As used herein, “additive manufacturing” refers to a manufacturing process in which materials are joined together in a process that repeatedly builds one layer on top of another to generate a three-dimensional structure or object. Additive manufacturing may also be referred to using different terms including additive processes, additive fabrication, additive techniques, additive layer manufacturing, layer manufacturing, freeform fabrication, ASTM F2792 (American Society for Testing and Materials), and 3D printing. Additive manufacturing can build the three-dimensional structure or object using computer-controlled equipment that applies successive layers of the material(s) based on a three-dimensional model that may be defined using Computer Aided Design (CAD) software. Additive manufacturing can use a variety of materials including polymers, thermoplastics, metals, ceramics, biochemicals, and the like. Additive manufacturing may provide unique benefits, as an implant together with the pores and/or lattices can be directly manufactured (without the need to generate molds, tool paths, perform any milling, and/or other manufacturing steps).

“Palpable feedback” refers to a type of feedback that can be felt. In one embodiment, palpable feedback can refer to feedback that is readily noticeable, tangible, or easily felt or perceived. During certain medical procedures, such as an osteotomy, palpable feedback can include the tactile sensations experienced by the healthcare provider as they move bones or bone fragments. (© ChatGPT 3.5 Version, Modified, accessed chat.openai.com/chat Feb. 2, 2024). Often palpable feedback is feedback a user, such as a surgeon, feels as they perform one or more steps or actions in a surgical procedure.

“Three-dimensional surface” refers to a surface defined by a collection of points that have three coordinates (x, y, z), where each point represents a location in space. In a medical context, a three-dimensional surface can include a surface of an implant or instrument that is customized for a particular purpose. In certain embodiments, a three-dimensional surface may be customized to fit anatomy of a patient or to accommodate handling by a user (e.g., a handle). (© ChatGPT 3.5 Version, Modified, accessed chat.openai.com/chat Feb. 2, 2024). In certain embodiments, of the present disclosure, a three-dimensional surface may be a surface on a side of an instrument that is specifically configured or customized to fit or match anatomy of a patient.

“Repository” refers to any data source or dataset that includes data or content. In one embodiment, a repository resides on a computing device. In another embodiment, a repository resides on a remote computing or remote storage device. A repository may comprise a file, a folder, a directory, a set of files, a set of folders, a set of directories, a database, an application, a software application, content of a text, content of an email, content of a calendar entry, and the like. A repository, in one embodiment, comprises unstructured data. A repository, in one embodiment, comprises structured data such as a table, an array, a queue, a look up table, a hash table, a heap, a stack, or the like. A repository may store data in any format including binary, text, encrypted, unencrypted, a proprietary format, or the like.

As used herein, “bevel” refers to an edge of a structure that is not perpendicular to the faces of the piece, the edge has a slope or slant or angled profile and can refer to a sloped surface. Often a cutting tool such as a blade or tooth can have a beveled edge that facilitates the cutting edge in cutting into a target material. “bevel” and “chamfer” can be used interchangeably herein. (Search “bevel” on Wikipedia.com May 17, 2021. CC-BY-SA 3.0 Modified. Accessed Aug. 4, 2021; search “bevel” on Merriam-Webster.com. Merriam-Webster, 2021. Web. Accessed 4 Aug. 2021. Modified; search “bevel” on wordhippo.com. WordHippo, 2021. Web. Accessed 4 Aug. 2021. Modified.)

As used herein, “registration” or “image registration” refers to a method, process, module, component, apparatus, and/or system that seeks to achieve precision in the alignment of two images. As used here, “image” may refer to either or both an image of a structure or object and another image or a model (e.g., a computer-based model or a physical model, in either two dimensions or three dimensions). In the simplest case of image registration, two images are aligned. One image may serve as the target image and the other as a source image; the source image is transformed, positioned, realigned, and/or modified to match the target image. An optimization procedure may be applied that updates the transformation of the source image based on a similarity value that evaluates the current quality of the alignment. An iterative procedure of optimization may be repeated until a (local) optimum is found. An example is the registration of CT and PET images to combine structural and metabolic information. Image registration can be used in a variety of medical applications: Studying temporal changes; Longitudinal studies may acquire images over several months or years to study long-term processes, such as disease progression. Time series correspond to images acquired within the same session (seconds or minutes). Time series images can be used to study cognitive processes, heart deformations and respiration; Combining complementary information from different imaging modalities. One example may be the fusion of anatomical and functional information.

Since the size and shape of structures vary across modalities, evaluating the alignment quality can be more challenging. Thus, similarity measures such as mutual information may be used; Characterizing a population of subjects. In contrast to intra-subject registration, a one-to-one mapping may not exist between subjects, depending on the structural variability of the organ of interest. Inter-subject registration may be used for atlas construction in computational anatomy. Here, the objective may be to statistically model the anatomy of organs across subjects; Computer-assisted surgery: in computer-assisted surgery pre-operative images such as CT or MRI may be registered to intra-operative images or tracking systems to facilitate image guidance or navigation. There may be several considerations made when performing image registration: The transformation model. Common choices are rigid, affine, and deformable transformation models. B-spline and thin plate spline models are commonly used for parameterized transformation fields. Non-parametric or dense deformation fields carry a displacement vector at every grid location; this may use additional regularization constraints. A specific class of deformation fields are diffeomorphisms, which are invertible transformations with a smooth inverse; The similarity metric. A distance or similarity function is used to quantify the registration quality. This similarity can be calculated either on the original images or on features extracted from the images. Common similarity measures are sum of squared distances (SSD), correlation coefficient, and mutual information. The choice of similarity measure depends on whether the images are from the same modality; the acquisition noise can also play a role in this decision. For example, SSD may be the optimal similarity measure for images of the same modality with Gaussian noise. However, the image statistics in ultrasound may be significantly different from Gaussian noise, leading to the introduction of ultrasound specific similarity measures.

Multi-modal registration may use a more sophisticated similarity measure; alternatively, a different image representation can be used, such as structural representations or registering adjacent anatomy; The optimization procedure. Either continuous or discrete optimization is performed. For continuous optimization, gradient-based optimization techniques are applied to improve the convergence speed.(Search “medical image computing” on Wikipedia.com Jun. 24, 2021. CC-BY-SA 3.0 Modified. Accessed Jun. 25, 2021.)

As used herein, a “resection” refers to a method, procedure, or step that removes tissue from another anatomical structure or body. A resection is typically performed by a surgeon on a part of a body of a patient. (Search “surgery” on Wikipedia.com May 26, 2021. CC-BY-SA 3.0 Modified. Accessed May 26, 2021.) In certain embodiments, a resection may remove little or no tissue and may in such circumstances also be referred to as an incision or a dissection. Resection may be used as a noun or a verb. In the verb form, the term is “resect” and refers to an act of performing, or doing, a resection. Past tense of the verb resect is resected.

“Resection feature” refers to any feature configured, designed, engineered and/or intended to facilitate resection. Examples of a resection guide feature include but are not limited to, a slot, a cut channel, a cut slot, a pivoting cut guide, a pivoting resection guide, an opening, a straight slot, an angled slot, a curved slot, or the like.

“Patient-matched” refers to a feature, aspect, attribute, characteristic, instrument, and/or device that is selected from a set of predetermined, predefined, precalculated, preconfigured, prearranged, and/or pre-fabricated structures, apparatuses, devices, instruments or devices to satisfactorily service a user based on a set of characteristics, such as size of an anatomical structure, deformity, fracture, laceration, opening, angles for certain landmarks, angles for a deformity, type of deformity, size of the bone, and the like. In certain embodiments, patient-matched is different from patient-specific.

“Bone condition” refers to any of a variety of conditions of bones of a patient. Generally, a bone condition refers to an orientation, position, and/or alignment of one or more bones of the patient relative to other anatomical structures of the body of the patient. Bone conditions may be caused by or result from deformities, misalignment, malrotation, fractures, joint failure, and/or the like. A bone condition includes, but is not limited to, any angular deformities of one or more bone segments in either the lower or upper extremities (for example, tibial deformities, calcaneal deformities, femoral deformities, and radial deformities). Alternatively, or in addition, “bone condition” can refer to the structural makeup and configuration of one or more bones of a patient. Thus bone condition may refer to a state or condition of regions, a thickness of a cortex, bone density, a thickness and/or porosity of internal regions (e.g. whether it is calcaneus or solid) of the bone or parts of the bone such as a head, a base, a shaft, a protuberance, a process, a lamina, a foramen, and the like of a bone, along the metaphyseal region, epiphysis region, and/or a diaphyseal region. “Malrotation” refers to a condition in which a part, typically a part of a patient's body has rotated from a normal position to an unnormal or uncommon position.

As used herein, a “guide” refers to a part, component, member, or structure designed, adapted, configured, or engineered to guide or direct one or more other parts, components, or structures. A guide may be part of, integrated with, connected to, attachable to, or coupled to, another structure, device, or instrument. In one embodiment, a guide may include a modifier that identifies a particular function, location, orientation, operation, type, and/or a particular structure of the guide. Examples of such modifiers applied to a guide, include, but are not limited to, “pin guide” that guides or directs one or more pins, a “cutting guide” that guides or directs the making or one or more cuts, a placement, deployment, or insertion guide that guides or directs the placement, positioning, orientation, deployment, installation, or insertion of a fastener and/or implant, a “cross fixation guide” that guides deployment of a fastener or fixation member, an “alignment guide” that guides the alignment of two or more objects or structures, a “resection guide” that serves to guide resection of soft or hard tissue, such as in an osteotomy, a “reduction guide” can serve to guide reduction of one or more bone segments or fragments, an “placement guide” that serves to identify how an object can be placed in relation to another object or structure, and the like. Furthermore, guides may include modifiers applied due to the procedure or location within a patient for which the guide is to be used. For example, where a guide is used at a joint, the guide may be referred to herein as an “arthrodesis guide”.

“Guide pin” refers to a pin, structure, or a type of fastener that can be used to guide an instrument or implant as part of a method, process, or procedure, such as a surgical technique. In certain aspects, a guide pin may be designed for temporary use until subsequent steps in a method, process, or procedure. Examples of a guide pin include, but are not limited to, a pin, a K-wire, and the like.

“Straight cut” is a type of cut that may be used for a surgical procedure. Generally, a straight cut is a cut in tissue (soft tissue or hard tissue) that is perpendicular to a surface where the cut is made and extends within and/or through the tissue along a straight line. Advantageously, straight cuts may be easier for a surgeon to perform than an angled cut or a curved cut. In certain embodiments, a straight cut is made with a guide (e.g., cut guide, resection guide, and/or resection feature). Alternatively, or in addition, a user may make a straight cut free-hand (without the aid of a guide, instrument, or instrumentation).

As used herein, “feature” refers to a distinctive attribute or aspect of something. (Search “feature” on google.com. Oxford Languages, 2021. Web. 20 Apr. 2021.) A feature may include one or more apparatuses, structures, objects, systems, sub-systems, devices, or the like. A feature may include a modifier that identifies a particular function or operation and/or a particular structure relating to the feature. Examples of such modifiers applied to a feature, include, but are not limited to, “attachment feature,” “securing feature,” “placement feature,” “protruding feature,” “engagement feature,” “disengagement feature,” “resection feature”, “guide feature”, and the like.

“Engagement feature” or “Engagement member” refers to an apparatus, instrument, structure, device, component, member, system, assembly or module structured, organized, configured, designed, arranged, or engineered to connect, join, link, couple to, or engage with another object, apparatus, instrument, structure, device, component, member, system, assembly or module either permanently or temporarily. The connection, coupling, linkage, or engagement may be a mechanical connection or interconnection.

“Cortical bone” refers to a type of bone tissue. Cortical bone is a type of bone tissue typically found between an external surface of a bone and an interior area of the bone. Cortical bone is more dense and typically stronger structurally than other types of bone tissue. “Cortical surface” refers to a surface of cortical bone.

“Transosseous placement feature” refers to a placement feature that extends through one or more bones and that enables, or facilitates, placement of another device, apparatus, or instrument.

“Patient-specific” refers to an attribute, aspect, feature, characteristic, function, structure, device, guide, tool, instrument, apparatus, member, component, system, assembly, module, or subsystem that is adjusted, tailored, modified, organized, configured, designed, arranged, engineered, and/or fabricated to specifically address the anatomy, physiology, condition, abnormalities, needs, or desires of a particular patient and/or surgeon serving the particular patient. In one aspect, a patient specific aspect or feature is unique to a single patient and may include features unique to the patient such as a number of cut channels, a number of bone attachment features, a number of bone engagement surfaces, a number of resection features, a depth of one or more cutting channels, an angle for one or more resection channels, a surface contour, component position, component orientation, and/or other features.

“Patient specific feature” refers to a feature, function, structure, device, guide, tool, instrument, apparatus, member, component, system, assembly, module, or subsystem that is adjusted, tailored, modified, organized, configured, designed, arranged, engineered, and/or fabricated to specifically address the anatomy, physiology, condition, abnormalities, needs, or desires of a particular patient or surgeon serving the particular patient. In one aspect, a patient specific feature is unique to a single patient and may include features unique to the patient such as a number of cut channels, a number of bone attachment features, a number of bone engagement surfaces, a number of resection features, a depth of one or more cutting channels, an angle for one or more resection channels, a surface contour, component position, component orientation, and/or other features. “Medial resection guide” refers to a resection guide designed, engineered, fabricated, or intended for use with, one, in, or about a medial part, section, surface, portion, or aspect of an anatomical structure such as a bone, digit, limb, or other anatomical structure for one or more steps of a resection procedure. “Lateral resection guide” refers to a resection guide designed, engineered, fabricated, or intended for use with, one, in, or about a lateral part, section, surface, portion, or aspect of an anatomical structure such as a bone, digit, limb, or other anatomical structure for one or more steps of a resection procedure.

“Bone fragment” refers to a part of a bone that is normally part of another bone of a patient. A bone fragment may be separate from another bone of a patient due to a deformity or trauma. In one aspect, the bone the bone fragment is normally connected or joined with is referred to as a parent bone.

“Orientation” refers to a direction, angle, position, condition, state, or configuration of a first object, component, part, apparatus, system, or assembly relative to another object, component, part, apparatus, system, assembly, reference point, reference axis, or reference plane.

“Longitudinal axis” refers to an axis of a structure, device, object, apparatus, or part thereof that extends from one end of a longest dimension to an opposite end. Typically, a longitudinal axis passes through a center of the structure, device, object, apparatus, or part thereof along the longitudinal axis. The center point used for the longitudinal axis may be a geometric center point and/or a mass center point.

Those of skill in the art will appreciate that a resection feature may take a variety of forms and may include a single feature or one or more features that together form the resection feature. In certain embodiments, the resection feature may take the form of one or more slots. Alternatively, or in addition, a resection feature may be referenced using other names including, but not limited to, channel, cut channels, and the like.

“Cutting tool” refers to any tool that can be used to cut or resect another object. In particular, a cutting tool can refer to a manual or power tool for cutting or resecting tissue of a patient. Examples of cutting tools include, but are not limited to, a burr, an oscillating saw, a reciprocating saw, a grater saw, a drill, a mill, a side-cutting burr, or the like.

“Revised model” refers to a model that has been changed from an original condition to an altered or changed or revised condition. Generally, an original model is used to create the revised model, alternatively, a revised model can be generated from scratch. Often the original model exists in a digital form on a computer. Such models can be referred to as CAD models.

“Corrected model” refers to a model in which an object, structure, and/or subject of the model has been changed from a deformed or incorrect configuration to a corrected or revised configuration. A corrected model can be generated from scratch or can be generated by revising an existing model and/or merging two or more models. As one example, an original model may represent one or more bones of a foot. The modeled bones of the foot may be subject to a bone condition and therefore have a deformity. A corrected model can be created or formed from the original model by revising or changing one or more aspects of the original model such that the modeled bones reflect a corrected orientation and or configuration for the modeled bones.

The present disclosure discloses methods, systems, and/or apparatuses for providing a physical model of a patient anatomical structure to a customer. In one embodiment, the physical model is a three-dimensional model of the patient anatomical structure. In certain embodiments, the patient anatomical structure is a foot, a foot and ankle, a hand, a hand and wrist, a shoulder, a knee, a neck or the like.

Medical care and medical technology continues to advance. Surgeons continue to find new ways to address patient's needs while minimizing the risks of an adverse outcome, the pain and discomfort for the patient, and the length of recovery while increasing the likelihood of desired outcomes. In particular, surgeons continue to work to make smaller incisions and perform surgical procedures in smaller spaces by way of minimally invasive surgical (MIS) procedures.

While MIS procedures can provide advantages for the patient, they can increase stress or present other challenges for the surgeon. What is needed is a tool that would provide a surgeon with an accurate representation of the hard tissue and/or soft tissue of a patient before, during, and/or a surgical procedure. Modern technologies enable a surgeon to visualize anatomical structures of a particular patient using two dimensional pictures, images, on paper or video screens. Other technologies enable a surgeon to see three dimensional representations of anatomical structures, again on computer screens or using augmented reality technologies.

While such technologies can be helpful they are not the same and do not provide the same advantages as providing a surgeon with a physical three dimensional model of one or more anatomical structures of a particular patient. Embodiments of the present disclosure a physical three-dimensional model of one or more anatomical structures of a particular patient to a customer, such as the patient, a facility, and/or a surgeon.

The present disclosure describes apparatuses, systems, and/or methods for generating and/or providing both patient-specific physical three-dimensional models and/or patient-specific instrumentation including instruments, guides, implants, and the like.

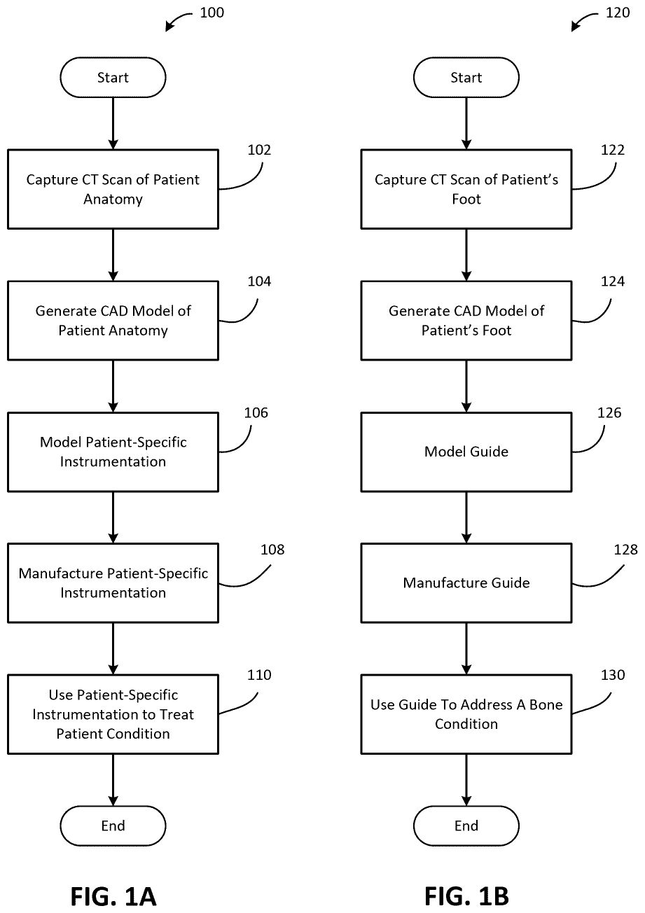

FIG. 1A is a flowchart diagram depicting a method 100 for correcting a bone condition, according to one embodiment. The method 100 may be used for any of a wide variety of bone conditions, including but not limited to deformities, fractures, joint failure, and/or the like. Further, the method 100 may provide correction with a wide variety of treatments, including but not limited to arthroplasty, arthrodesis, fracture repair, and/or the like.

As shown, the method 100 may begin with a step 102 in which a CT scan (or another three-dimensional image, also referred to as medical imaging) of the patient's anatomy is obtained. The step 102 may entail capturing a scan of only the particular bone(s) to be treated, or may entail capture of additional anatomic information, such as the surrounding tissues. Additionally, or alternatively, the step 102 may entail receiving a previously captured image, for example, at a design and/or fabrication facility. Performance of the step 102 may result in possession of a three-dimensional model of the patient's anatomy, or three-dimensional surface points that can be used to construct such a three-dimensional model.

After the step 102 has been carried out, the method 100 may proceed to a step 104 in which a CAD model of the patient's anatomy (including one or more bones) is generated. The CAD model may be one example of a bone model. The CAD model may be of any known format, including but not limited to SolidWorks, Catia, AutoCAD, or DXF. In some embodiments, customized software may be used to generate the CAD model from the CT scan. The CAD model may only include the bone(s) to be treated and/or may include surrounding tissues. In alternative embodiments, the step 104 may be omitted, as the CT scan may capture data that can directly be used in future steps without the need for conversion.

In one embodiment, the CAD model generated and/or patient-specific instrumentation, implants, and/or plan for conducting an operative procedure, may be enhanced by the use of advanced computer analysis system, machine learning, and/or automated/artificial intelligence. For example, these technologies may be used to revise a set of steps for a procedure such that a more desirable outcome is achieved.

In a step 106, the CAD model and/or CT scan data may be used to model patient-specific instrumentation that can be used to correct the condition, as it exists in the patient's anatomy. In some embodiments, any known CAD program may be used to view and/or manipulate the CAD model and/or CT scan and generate one or more instruments that are matched specifically to the size and/or shape of the patient's bone(s). In some embodiments, such instrumentation may include a targeting guide, trajectory guide, drill guide, cutting guide, tendon trajectory guide, positioner or positioning guide or similar guide that can be attached to one or more bones, with one or more features that facilitate work on the one or more bones pursuant to a procedure such as arthroplasty or arthrodesis. In some embodiments, performance of the step 106 may include modelling an instrument with a bone engagement surface that is shaped to match the contour of a surface of the bone, such that the bone engagement surface can lie directly on the corresponding contour.

In a step 108, the model(s) may be used to manufacture patient-specific instrumentation and/or implants. This may be done via any known manufacturing method, including casting, forging, milling, additive manufacturing, and/or the like. Additive manufacturing may provide unique benefits, as the model may be directly used to manufacture the instrumentation and/or implants (without the need to generate molds, tool paths, and/or the like beforehand). Such instrumentation may optionally include a targeting guide, trajectory guide, drill guide, cutting guide, positioning guide, positioner, or tendon trajectory guide with the bone engagement surface and one or more features as described herein.

In addition to, or in the alternative to the step 108, the model(s) may be used to select from available sizes of implants and/or instruments and advise the surgeon accordingly. For example, where a range of guides are available for a given procedure, analysis of the CAD data may facilitate pre-operative selection of the optimal guide and/or optimal placement of the guide on the bone. Similarly, if a range of implants may be used for a given procedure, analysis of the CAD data may facilitate pre-operative selection of the optimal implant(s). More particularly, properly-sized spacers, screws, bone plates, and/or other hardware may be pre-operatively selected.

Thus, the result of the step 108 may provision, to the surgeon, of one or more of the following: (1) one or more patient-specific instruments; (2) one or more patient-specific implants; (3) an instrument, selected from one or more available instrument sizes and/or configurations; (4) an implant, selected from one or more available implant sizes and/or configurations; (5) instructions for which instrument(s) to select from available instrument sizes and/or configurations; (6) instructions for which implant(s) to select from available implant sizes and/or configurations; (7) instructions for proper positioning or anchorage of one or more instruments to be used in the procedure; and (8) instructions for proper positioning or anchorage of one or more implants to be used in the procedure. These items may be provided to the surgeon directly, or to a medical device company or representative, for subsequent delivery to the surgeon.

In a step 110, the manufactured instrumentation may be used in surgery to facilitate treatment of the condition. In some embodiments, this may entail placing the modelled bone engagement surface against the corresponding contour of the bone used to obtain its shape, and then using the resection feature(s) to guide resection of one or more bones. Then the bone(s) may be further treated, for example, by attaching one or more joint replacement implants (in the case of joint arthroplasty), or by attaching bone segments together (in the case of arthrodesis or fracture repair). Prior to completion of the step 110, the instrumentation may be removed from the patient, and the surgical wound may be closed.

As mentioned previously, the method 100 may be used to correct a wide variety of bone conditions. One example of the method 100 will be shown and described in connection with FIG. 1B, for correction of a bunion deformity of the foot.

In certain embodiments, one or more of a method, apparatus, and/or system of the disclosed solution can be used for training a surgeon to perform a patient-specific procedure or technique. In one embodiment, the CAD model generated and/or patient-specific instrumentation, implants, and/or plan for conducting an operative procedure can be used to train a surgeon to perform a patient-specific procedure or technique.

In one example embodiment, a surgeon may submit a CT scan of a patient's foot to an apparatus or system that implements the disclosed solution. Next, a manual or automated process may be used to generate a CAD model and for making the measurements and correction desired for the patient. In the automated process, advanced computer analysis system, machine learning and automated/artificial intelligence may be used to generate a CAD model and/or one or more patient-specific instruments and/or operation plans. For example, a patient-specific guide may be fabricated that is registered to the patient's anatomy using a computer-aided machine (CAM) tool. In addition, a CAM tool may be used to fabricate a 3D structure representative of the patient's anatomy, referred to herein as a patient-specific synthetic cadaver. (e.g. one or more bones of a patient's foot). Next, the patient-specific guide and the patient-specific synthetic cadaver can be provided to a surgeon who can then rehearse an operation procedure in part or in full before going into an operating room with the patient.

In certain embodiments, the patient-specific guide or instrument can be used to preposition and/or facilitate pre-drilling holes for a plate system for fixation purposes. Such plate systems may be optimally placed, per a CT scan, after a correction procedure for optimal fixation outcome. In another embodiment, the CAD model and/or automated process such as advanced computer analysis, machine learning and automated/artificial intelligence may be used to measure a depth of the a through a patient-specific resection guide for use with robotics apparatus and/or systems which would control the depth of each cut within the guide to protect vital structures below or adjacent to a bone being cut. In another embodiment, the CAD model and/or automated process such as advanced computer analysis, machine learning and automated/artificial intelligence may be used to define desired fastener (e.g. bone screw) length and/or trajectories through a patient-specific guide and/or implant. The details for such lengths, trajectories, and components can be detailed in a report provided to the surgeon preparing to perform a procedure.

FIG. 1B is a flowchart diagram depicting a method 120 for correcting or remediating a bone condition, according to one embodiment. The method 120 may be used to prepare for an orthopedic procedure which corrects or remediates a bone, muscle, and/or tendon condition of a patient.

As shown, the method 120 may begin with a step 122 in which a CT scan (or another three-dimensional image) of the patient's foot is obtained. The step 122 may entail capturing a scan of only the first cuneiform and first metatarsal, or may entail capture of additional anatomic information, such as the entire foot. Additionally or alternatively, the step 122 may entail receipt of previously captured image data. Capture of the entire foot in the step 122 may facilitate proper alignment of the first metatarsal with the rest of the foot (for example, with the second metatarsal). Performance of the step 122 may result in generation of a three-dimensional model of the patient's foot, or three-dimensional surface points that can be used to construct such a three-dimensional model.

After the step 122 has been carried out, the method 120 may proceed to a step 124 in which a CAD model of the relevant portion of the patient's anatomy is generated. The CAD model may optionally include the bones of the entire foot, like the CT scan obtained in the step 122. In alternative embodiments, the step 124 may be omitted in favor of direct utilization of the CT scan data, as described in connection with the step 104.

In a step 126, the CAD model and/or CT scan data may be used to model patient-specific instrumentation that can be used to correct or remediate a bone condition. Such instrumentation may include a guide. In one example, the guide can seat or abut or contact a surface of a bone and including an opening that guides a trajectory for a fastener for a procedure. In some embodiments, performance of the step 126 may include modelling the guide with a bone engagement surface that is shaped to match contours of the surfaces of the bone, such that the bone engagement surface can lie directly on the corresponding contours of the bone.

In a step 128, the model(s) may be used to manufacture patient-specific instrumentation and/or instruments. This may include manufacturing the guide with the bone engagement surface and/or other features as described above. As in the step 108, the step 128 may additionally or alternatively involve provision of one or more instruments and/or implants from among a plurality of predetermined configurations or sizes. Further, the step 128 may additionally, or alternatively, involve provision of instructions for placement and/or anchorage of one or more instruments and/or instruments to carry out the procedure.

In a step 130, the manufactured guide may be used in surgery to facilitate treatment of the condition. Specifically, the bone engagement surface of the guide may be placed against the corresponding contours of the bone. The guide may include an opening and/or trajectory guide to guide insertion of a trajectory guide such as a temporary fastener such as a K-wire. The guide may then be removed, and the remaining steps of a surgical procedure performed.

The method 100 and the method 120 are merely exemplary. Those of skill in the art will recognize that various steps of the method 100 and the method 120 may be reordered, omitted, and/or supplemented with additional steps not specifically shown or described herein.

As mentioned previously, the method 120 is one species of the method 100; the present disclosure encompasses many different procedures, performed with respect to many different bones and/or joints of the body. Exemplary steps and instrumentation for the method 120 will further be shown and described in connection with the present disclosure. Those of skill in the art will recognize that the method 120 may be used in connection with different instruments; likewise, the instruments of the present disclosure may be used in connection with methods different from the method 100 and the method 120.

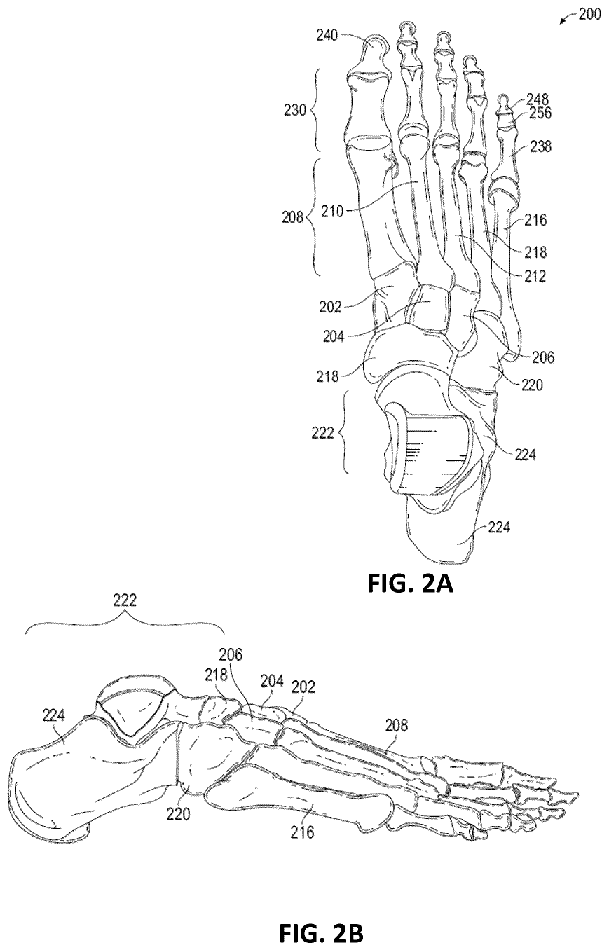

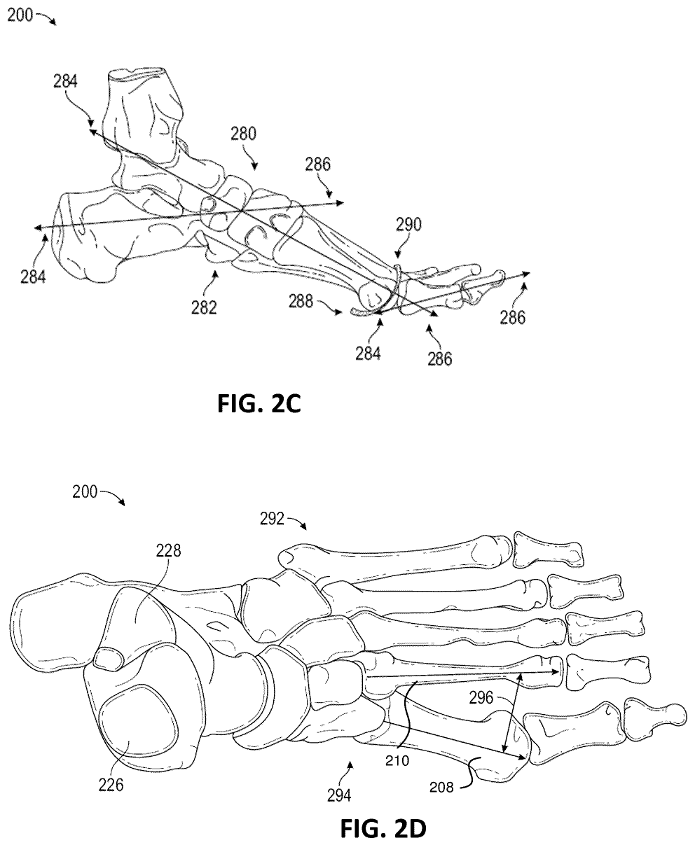

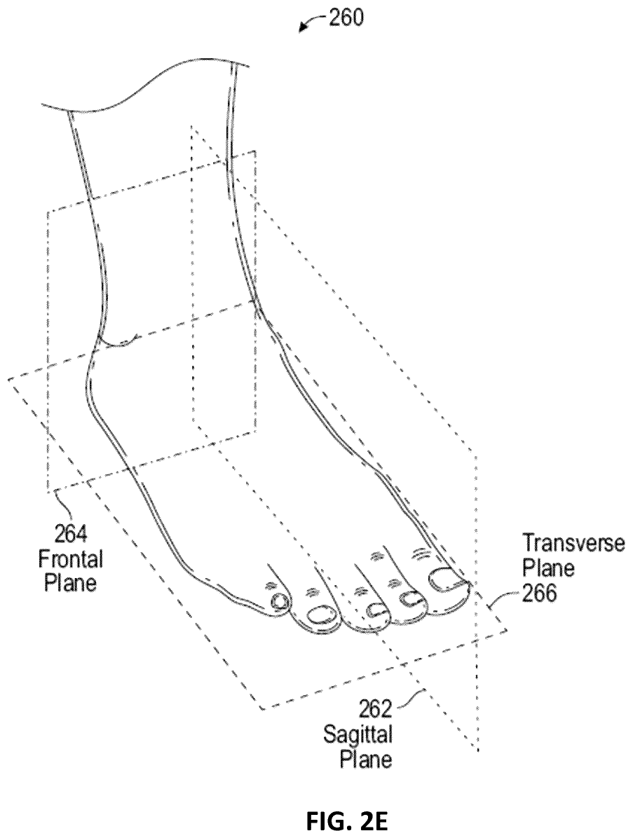

FIG. 2A is a perspective dorsal view of a foot 200. The foot 200 may have a medial cuneiform 202, an intermediate cuneiform 204, lateral cuneiform 206, a first metatarsal 208, a second metatarsal 210, third metatarsal 212, fourth metatarsal 214, fifth metatarsal 216, navicular 218, cuboid 220, and calcaneus 222, among others. The medial cuneiform 202 and the intermediate cuneiform 204 may be joined together at a first metatarsocuneiform joint, and the first metatarsal 208 and the second metatarsal 210 may be joined together at a second metatarsocuneiform joint.

FIG. 2B is a perspective lateral view of a foot 200, with bones of the foot labeled.

FIG. 2C is a perspective medial view of a foot illustrating a dorsal side 280 and a plantar side 282. Dorsal refers to the top of the foot. Plantar refers to the bottom of the foot. Proximal 284 is defined as “closer to the primary attachment point”. Distal 286 is defined as “further away from the attachment point”. Plantarflex or plantarflexion 288 means movement toward the plantar side 282 of a foot or hand, toward the sole or palm. Dorsiflex or dorsiflexion 290 means movement toward the dorsal side 280 of a foot or hand, toward the top. FIG. 2D is a perspective dorsal view of the foot 200. A transverse plane is the plane that shows the top of the foot. A lateral side 292 means a side furthest away from the midline of a body, or away from a plane of bilateral symmetry of the body. A medial side 294 means a side closest to the midline of a body, or toward a plane of bilateral symmetry of the body. For a Lapidus procedure, the intermetatarsal (IM) angle 296 is the angle to be corrected to remove the hallux valgus (bunion) deformity. The IM angle 296 is an angle between the a longitudinal axis of a first metatarsal and a second metatarsal of a patient.

FIG. 2E is a view of a foot illustrating common planes 260 of reference for a human foot. FIG. 2E illustrates a sagittal plane 262 that divides the foot into a right section and a left section half. The sagittal plane 262 is perpendicular to frontal or coronal plane 264 and the transverse plane 266. In the foot, the frontal plane 264 generally runs vertically through the ankle and the transverse plane 266 generally runs horizontally through the midfoot and toes of the foot.

Every patient and/or condition is different; accordingly, the degree of angular adjustment needed in each direction may be different for every patient. Use of a patient-specific guide may help the surgeon obtain an optimal realignment, target, or position a bone tunnel, position one or more resections and/or fasteners and the like. Thus, providing patient-specific guides, jigs, and/or instrumentation may provide unique benefits.

The present patient-specific instrumentation may be used to correct a wide variety of bone conditions. Such conditions include, but are not limited to, any angular deformities from within one bone segment in either the lower or upper extremities (for example, tibial deformities, calcaneal deformities, femoral deformities, and radial deformities). The present disclosure may also be used to treat an interface between two bone segments (for example, the ankle joint, metatarsal cuneiform joint, lisfranc's joint, complex Charcot deformity, wrist joint, knee joint, etc.). As one example, an angular deformity or segmental malalignment in the forefoot may be treated, such as is found at the metatarsal cuneiform level, the midfoot level such as the navicular cuneiform junction, hindfoot at the calcaneal cuboid or subtalar joint or at the ankle between the tibia and talar junction. Additionally, patient-specific instruments could be used in the proximal leg between two bone segments or in the upper extremity such as found at the wrist or metacarpal levels.

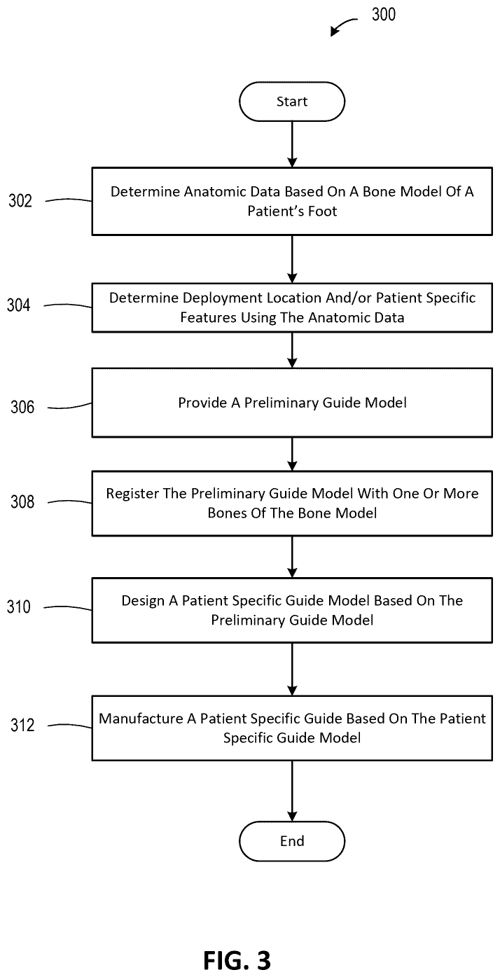

FIG. 3 illustrates a flowchart diagram depicting a method 300 for generating one or more patient-specific instruments configured to correct or address a bone or foot condition, according to one embodiment. Prior to steps of the method 300, a bone model (also referred to as CAD model above) is generated. The bone model may be generated using medical imaging of a patient's foot and may also be referred to as an anatomic model. The medical imaging image(s) may be used by computing devices to generate patient imaging data. The patient imaging data may be used to measure and account for orientation of one or more structures of a patient's anatomy. In certain embodiments, the patient imaging data may serve, or be a part of, anatomic data for a patient.

In one embodiment, the method 300 begins after a bone model of a patient's body or body part(s) is generated. In a first step 302, the method 300 may review the bone model and data associated with the bone model to determine anatomic data of a patient's foot.

After step 302, the method 300 may determine 304 a recommended location and/or a trajectory angle and/or patient-specific features for a procedure using the anatomic data. “Recommended location” refers to a location for deployment of guide or instrument on, in, between, or within one or more body parts (e.g., bones) of a patient. “Trajectory angle” refers to a recommended angle for deployment of an instrument, graft, body part, or resection feature angle relative to a bone of a patient for a procedure. In certain embodiments, determining the recommended location may employ advanced computer analysis system, expert systems, machine learning, and/or automated/artificial intelligence. In another embodiment, the method 300 may include determining one or more alternative locations and/or trajectory angles for instrumentation.

Next, the method 300 may proceed and a preliminary guide model is provided 306 from a repository of template instrumentation models. A preliminary guide model is a model of a preliminary guide.

As used herein, “preliminary guide” refers to a guide configured, designed, and/or engineered to serve as a template, prototype, archetype, or starting point for creating, generating, or fabricating a patient-specific guide. In one aspect, the preliminary guide may be used, as-is, without any further changes, modifications, or adjustments and thus become a patient-specific guide. In another aspect, the preliminary guide may be modified, adjusted, or configured to more specifically address the goals, objectives, or needs of a patient or a surgeon and by way of the modifications become a patient-specific guide. The patient-specific guide can be used by a user, such as a surgeon, to guide steps in a surgical procedure, such as an osteotomy. Accordingly, a preliminary guide model can be used to generate a patient-specific guide. The patient-specific guide model may be used in a surgical procedure to facilitate one or more steps of the procedure and may be used to generate a patient-specific guide that can be used in a surgical procedure for the patient.

In certain embodiments, the preliminary guide model may be generated based on anatomic data and/or a bone model or a combination of these, and no model or predesigned structure, template, or prototype. Alternatively, or in addition, the preliminary guide model may be, or may originate from, a template guide model selected from a set of template guide models. Each model in the set of template guide models may be configured to fit for an average patient's foot. The template guide model may subsequently be modified or revised by an automated process or manual process to generate the preliminary guide model used in this disclosure.

As used herein, “template guide” refers to a guide configured, designed, and/or engineered to serve as a template for creating, generating, or fabricating a patient-specific guide. In one aspect, the template guide may be used, as-is, without any further changes, modifications, or adjustments and thus become a patient-specific guide. In another aspect, the template guide may be modified, adjusted, or configured to more specifically address the goals, objectives, or needs of a patient or a surgeon and by way of the modifications become a patient-specific guide. The patient-specific guide can be used by a user, such as a surgeon, to guide making one or more resections of a structure, such as a bone for a procedure. Accordingly, a template guide model can be used to generate a patient-specific guide model. The patient-specific guide model may be used in a surgical procedure to address, correct, or mitigate effects of the identified deformity and may be used to generate a patient-specific guide that can be used in a surgical procedure for the patient.

Next, the method 300 may register 308 the preliminary guide model with one or more bones of the bone model. This step 308 facilitates customization and modification of the preliminary guide model to generate a patient-specific guide model from which a patient-specific guide can be generated. The registration step 308 may combine two models and/or patient imaging data and positions both models for use in one system and/or in one model.

Next, the method 300 may design 310 a patient-specific guide model based on the preliminary guide model. The design step 310 may be completely automated or may optionally permit a user to make changes to a preliminary guide model or partially completed patient-specific guide model before the patient-specific guide model is complete. A preliminary guide model and patient-specific guide model are two examples of an instrument model. As used herein, “instrument model” refers to a model, either physical or digital, that represents an instrument, tool, apparatus, or device. Examples of an instrument model can include a cutting guide model, a resection guide model, an alignment guide model, a reduction guide model, a patient-specific tendon trajectory guide model, a positioner model, a positioning guide model, and the like. In one embodiment, a patient-specific guide and a patient-specific guide model may be unique to a particular patient and that patient's anatomy and/or condition.

The method 300 may conclude by a step 312 in which patient-specific guide may be manufactured based on the patient-specific guide model. Various manufacturing tools, devices, systems, and/or techniques can be used to manufacture the patient-specific guide.

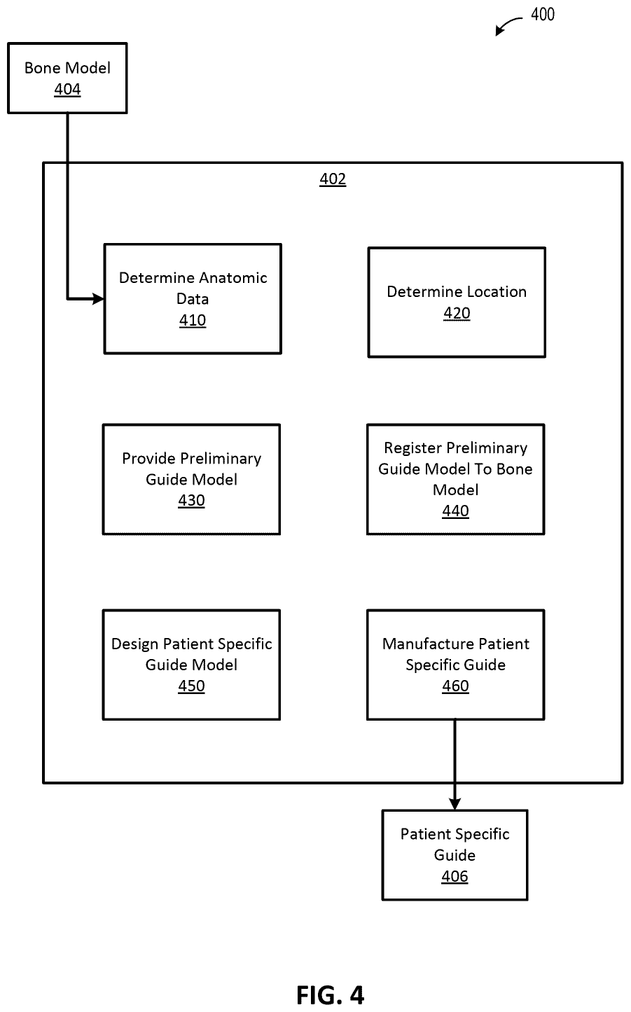

FIG. 4 illustrates an exemplary system 400 configured to generate one or more patient-specific instruments configured to facilitate surgical procedures, according to one embodiment. The system 400 may include an apparatus 402 configured to accept, review, receive or reference a bone model 404 and provide a patient-specific guide 406. In one embodiment, the apparatus 402 is a computing device. In another embodiment, the apparatus 402 may be a combination of computing devices and/or software components or a single software component such as a software application.

The apparatus 402 may include a determination module 410, a location module 420, a provision module 430, a registration module 440, a design module 450, and a manufacturing module 460. Each of which may be implemented in one or more of software, hardware, or a combination of hardware and software.

The determination module 410 determines anatomic data 412 from a bone model 404. In certain embodiments, the system 400 may not include a determination module 410 if the anatomic data is available directly from the bone model 404. In certain embodiments, the anatomic data for a bone model 404 may include data that identifies each anatomic structure within the bone model 404 and attributes about the anatomic structure. For example, the anatomic data may include measurements of the length, width, height, and density of each bone in the bone model. Furthermore, the anatomic data may include position information that identifies where each structure, such as a bone is in the bone model 404 relative to other structures, including bones. The anatomic data may be in any suitable format and may be stored separately or together with data that defines the bone model 404.

In one embodiment, the determination module 410 may use advanced computer analysis system such as image segmentation to determine the anatomic data. The determination module 410 may determine anatomic data from one more sources of medical imaging data, images, files, or the like. Alternatively, or in addition the determination module 410 may use software and/or systems that implement one or more artificial intelligence methods (e.g., machine learning and/or neural networks) for deriving, determining, or extrapolating, anatomic data from medical imaging or the bone model. In one embodiment, the determination module 410 may perform an anatomic mapping of the bone model 404 to determine each unique aspect of the intended osteotomy procedure and/or bone resection and/or bone translation. The anatomic mapping may be used to determine coordinates to be used for an osteotomy procedure, position and manner of resections to be performed either manually or automatically or using robotic surgical assistance, a width for bone cuts, an angle for bone cuts, a predetermined depth for bone cuts, dimensions and configurations for resection instruments such as saw blades, milling bit size and/or speed, saw blade depth markers, and/or instructions for automatic or robotic resection operations.

In one embodiment, the determination module 410 may use advanced computer analysis system such as image segmentation to determine the anatomic data. The determination module 410 may determine anatomic data from one more sources of medical imaging data, images, files, or the like. The determination module 410 may perform the image segmentation using 3D modeling systems and/or artificial intelligence (AI) segmentation tools. In certain embodiments, the determination module 410 is configured to identify and classify portions of bone based on a condition of the bone, based on the bone condition. Such classifications may include identifying bone stability, bone density, bone structure, bone deformity, bone structure, bone structure integrity, and the like. Accordingly, the determination module 410 may identify portions or sections or one or more bones based on a quality metric for the bone. Advantageously, that determination module 410 can identify high quality bone having a viable structure, integrity, and/or density versus lower quality bone having a nonviable structure, integrity, and/or density and a plurality of bone quality levels in between.

Accordingly, the determination module 410 can guide a surgeon to determine which areas of one or more bones of a patient are within a “soft tissue envelope” (bone of undesirable quality) as that bone relates to a particular deformity or pathology. Identifying the quality of one or more bones of the patient can aid a surgeon in determining what type of correction or adjustment is needed. For example, an ulceration that occurs due to a boney deformity can be mapped using the determination module 410 in a way that a correction can be performed to correct the deformity and reduce pressure to an area and address the structures that were causing the pressure ulceration/skin breakdown.

In addition, the determination module 410 and/or another component of the apparatus 402 can be used to perform anatomic mapping which may include advanced medical imaging, such as the use of CT scan, ultrasound, MRI, and bone density scans can be combined to effectively create an anatomic map that determines the structural integrity of the underlying bone.

Identifying the structural integrity of the underlying bone can help in determining where bone resections can be performed to preserve the densest bone in relation to conditions such as Charcot neuropathic, arthropathy where lesser dense bone can fail and collapse. It is well documented in the literature that failure to address and remove such lesser dense bone can ultimately lead to failure of a reconstruction and associated hardware.

The present disclosure provides, by way of at least the exemplary system 400, an anatomic map that can be part of anatomic data. The anatomic map can combine structural, deformity, and bone density information and can be utilized to determine the effective density of bone and help to determine where bone should be resected in order to remove the lesser dense bone while maintaining more viable bone to aid in the planning of the osteotomy/bone resection placement.

The location module 420 determines or identifies one or more recommended locations and/or trajectory angles for deployment of an instrument, graft, and/or soft tissue based on the anatomic data 412 and/or the bone model 404. In one embodiment, the location module 420 may compare the anatomic data 412 to a general model that is representative of most patient's anatomies and may be free from deformities or anomalies. The location module 420 can operate autonomously and/or may facilitate input and/or revisions from a user. The location module 420 may be completely automated, partially automated, or completely manual. A user may control how automated or manual the determining of the location and/or trajectory angles is.

The provision module 430 is configured to provide a preliminary guide model 438. The provision module 430 may use a variety of methods to provide the preliminary guide model. In one embodiment, the provision module 430 may generate a preliminary guide model. In the same, or an alternative embodiment, the provision module 430 may select a template guide model for a surgical procedure configured to enable locating the position for one or more instruments and/or providing a trajectory provided by the location module 420. In one embodiment, the provision module 430 may select a template guide model from a set of template guide models (e.g., a library, set, or repository of template guide models).

The registration module 440 registers the preliminary guide model with one or more bones or other anatomical structures of the bone model 404. As explained above, registration is a process of combining medical imaging data, patient imaging data, and/or one or more models such that the preliminary guide model can be used with the bone model 404.

The design module 450 designs a patient-specific guide (or patient-specific guide model) based on the preliminary guide model. The design operation of the design module 450 may be completely automated, partially automated, or completely manual. A user may control how automated or manual the designing of the patient-specific guide (or patient-specific guide model) is.

The manufacturing module 460 may manufacture a patient-specific guide 406 using the preliminary guide model. The manufacturing module 460 may use a patient-specific guide model generated from the preliminary guide model. The manufacturing module 460 may provide the patient-specific guide model to one or more manufacturing tools and/or fabrication tool. The patient-specific guide model may be sent to the tools in any format such as an STL file or any other CAD modeling or CAM file or method for data exchange. In one embodiment, a user can adjust default parameters for the patient-specific guide such as types and/or thicknesses of materials, dimensions, and the like before the manufacturing module 460 provides the patient-specific guide model to a manufacturing tool.

Effective connection of the guide to one or more bones can ensure that surgical steps are performed in desired locations and/or with desired orientations and mitigate undesired surgical outcomes.

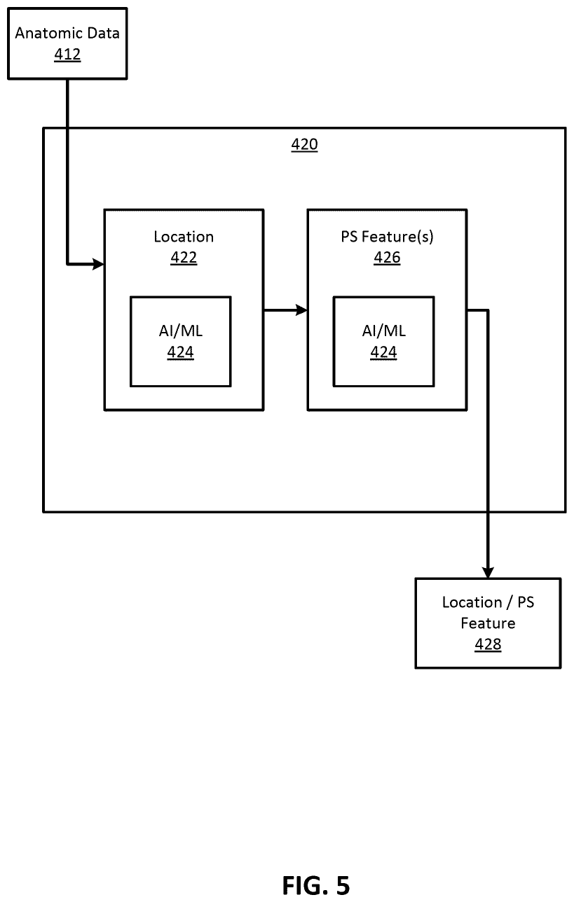

FIG. 5 illustrates an exemplary location module 420 configured to determine a recommended location and/or trajectory for steps and/or instruments in a surgical procedure, according to one embodiment. The location module 420 may factor in one or more landmarks on one or more surfaces of one or more bones of a patient of the bone model 404. The location module 420 may be completely automated, partially automated, or completely manual. A user may control how automated or manual the determination of the recommended location is. The user may provide instructions to the location module 420 to facilitate automatic or partially automated determination of one or more recommended locations.

The location module 420 may include a location module 422. The location module 422 may be configured for automated determination of a recommended location for steps and/or instruments in a surgical procedure. For example, in one embodiment, the location module 422 includes an artificial intelligence or machine learning module 424. The artificial intelligence or machine learning module 424 is configured to implement one or more of a variety of artificial intelligence modules that may be trained for identifying bones in the bone model 404, determining surfaces and/or sides of one or more bones, determining landmarks (both natural and/or abnormalities), determining axes of a bone, such as a longitudinal axis and/or a horizontal axis of a bone based on anatomic data 412 and/or a bone model 404. In another embodiment, the location module 420 may receive patient imaging data, a bone model, a CAD model or the like and use these inputs to determine a recommended location and/or trajectory in relation to one or more bones of a patient.

In one embodiment, the artificial intelligence or machine learning module 424 may be trained using a large data set of anatomic data 412 for healthy bones and a large data set of anatomic data 412 for bones with abnormalities and/or landmarks in which the abnormalities and/or landmarks have been previously identified and labeled in the dataset. The artificial intelligence or machine learning module 424 may implement, or use, a neural network configured according to the training such that as the artificial intelligence or machine learning module 424 accepts the anatomic data 412 for a particular patient, the artificial intelligence or machine learning module 424 is able to determine what one or more locations (e.g., a recommended location and one or more alternative locations for the guide.

The location module 422 may interact with a patient specific feature module 426. The patient specific feature module 426 may take one or more locations provided by the location module 422 and the bone model 404 and/or anatomic data 412 and determine suitable patient specific features. In certain embodiments, the patient specific features provided by the patient specific feature module 426 may include a number of resection features, an angle or trajectory for one or more resection features, a number, size, and/or position of bone attachment features, a number, size, or position of alignment guides or a combination of these. In certain embodiments, the patient specific feature module 426 may focus on resection features.

As with the location module 420, the patient specific feature module 426 may be completely automated, partially automated, or completely manual. A user may control how automated or manual the determination of the trajectory is. The user may provide instructions to the patient specific feature module 426 to facilitate automatic or partially automated determination of one or more trajectories. In one embodiment, the location module 422 includes an artificial intelligence or machine learning module 424 that facilitates determining one or more trajectories.

The location module 420 outputs a location/patient specific feature 428 for an orthopedic surgical procedure.

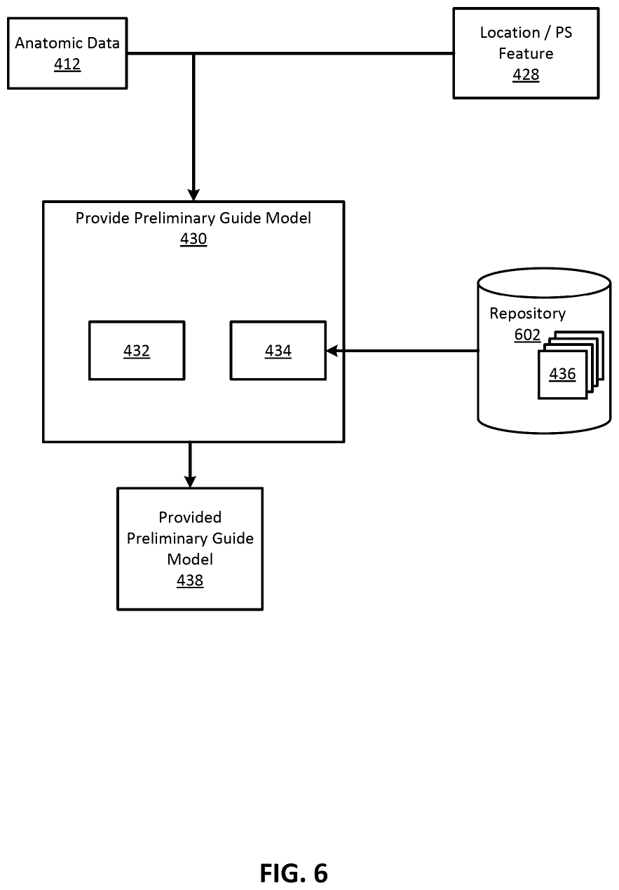

FIG. 6 illustrates an exemplary provision module 430 configured to provide a preliminary guide model, according to one embodiment. The provision module 430 may accept anatomic data 412 and a location/patient specific feature 428. In the illustrated embodiment, the provision module 430 may generate a preliminary guide model 438 (e.g., generate from ‘scratch’) or the provision module 430 may select a template guide model 436 automatically from a set of template guide models 436 stored in a repository 602. The provision module 430 may incorporate a variety of parameters in order to provision, generate, determine, or select a template guide model 436. For example, in addition to the anatomic data 412, the provision module 430 may include patient imaging data, deformity parameters for a variety of angular deformities (in all 3 planes) of the midfoot or hind foot and ankle, patient preferences, and/or surgeon input parameters.

In one embodiment, the provision module 430 may include a generator 432 and/or a selection module 434. In one embodiment, the generator 432 is configured to generate a preliminary guide model 438. In certain embodiments, the generator 432 may generate or create the preliminary guide model based on anatomic data and/or a bone model or a combination of these and no other inputs. (e.g. no model or predesigned structure, template, or prototype). Alternatively, or in addition, the generator 432 may generate or create the preliminary guide model using a standard set of features or components that can be combined to form the preliminary guide model. The generated preliminary guide model may subsequently be modified or revised by an automated process, and/or manual process, to generate the preliminary guide model used in this disclosure.

The selection module 434 may be configured to select a template guide model 436 for an osteotomy procedure configured to correct the deformity identified by the location module 420. In one embodiment, the provision module 430 may select a template guide model 436 from a set of template guide models 436 (e.g., a library, set, or repository of template guide models 436). In one embodiment, the template guide model 436 may include digital models. In another embodiment, the template guide model 436 may include physical models. In such an embodiment, the repository 602 may be a warehouse or other inventory repository. Where the template guide model 436 are physical models, the systems, modules, and methods of this disclosure can be used and the physical model may be milled or machined (e.g., a CNC machine) to form a patient-specific guide that conforms to the bone surfaces of the patient.

Selection of a suitable template guide model 436 may be completely automated and/or may be partially automated and/or may depend on confirmation from a user before a generated preliminary guide model or a proposed template guide model 436 becomes the preliminary guide model 438. In another embodiment, the selection module 434 may facilitate a manual selection by a user of a template guide model 436 that would become the preliminary guide model 438. The selection module 434 may use the anatomic data 412 or the bone model 404 or a combination of these to select a suitable template guide model that would become the preliminary guide model 438.

In another embodiment, the generator 432 may facilitate revisions or edits by a user of a generated guide model that will become the preliminary guide model 438. The selection module 434 may use the anatomic data 412 or the bone model 404 or a combination of these to select a suitable template guide model that would become the preliminary guide model 438.

The repository 602 may include any number of, and/or a variety of template guide models 436. The template guide models 436 may be distinguished based on a gender or age of the patient, which joint of a midfoot, hind foot, or ankle will be cut, which material will be used for the template guide, and the like. The template guide model 436 may differ from each other in what degree of deformity correction the template guide model 436 is designed to provide. In addition, the template guide models 436 may be distinguished based on how one or more features of the template guide model 436 are positioned, arranged, and/or configured relative to each other. For example, in certain template guide models 436, the number, position, and/or configuration of alignment features and/or bone attachment features (e.g., holes) may vary based on needs or preferences of patients, the nature of the deformity, and/or surgeon preferences.

In certain embodiments, the template guide models 436 may vary in how the slots (e.g., resection features) for the cuts are positioned, angled, and oriented relative to each other and/or to a longitudinal axis of respective bones at a joint for use with the template guide model 436. For example, in one template guide model 436 the slot 1352 for a resection of a metatarsal bone may be perpendicular to a longitudinal axis of the metatarsal bone and the slot 1350 may be angled relative to a longitudinal axis of the resection or cuboid bone such that once the two bones are brought together the deformity is corrected. Alternatively, in another template guide model 436 the slot for a resection of a metatarsal bone may be angled relative to a longitudinal axis of the metatarsal bone and the slot 1350 may be perpendicular to a longitudinal axis of the cuneiform or cuboid bone such that once the two bones are brought together the deformity is corrected.

The selection module 434 may be configured to automatically select a template guide model 436 and/or provide an automatic template guide model 436 recommendation that can be changed by a user, such as a surgeon. For example, in one embodiment, the provision module 430 and/or selection module 434 includes an artificial intelligence or machine learning module. The artificial intelligence or machine learning module is configured to implement one or more of a variety of artificial intelligence modules that may be trained for selecting a template guide model 436 based on anatomic data 412 and/or other input parameters. In one embodiment, the artificial intelligence or machine learning module may be trained using a large data set of anatomic data 412 for suitable template guide models 436 identified and labeled in the dataset by professionals for use to treat a particular deformity. The artificial intelligence or machine learning module may implement, or use, a neural network configured according to the training such that as the artificial intelligence or machine learning module is able to select a suitable template guide model 436. The template guide model 436 selected by the selection module 434 can become the preliminary guide model 438.

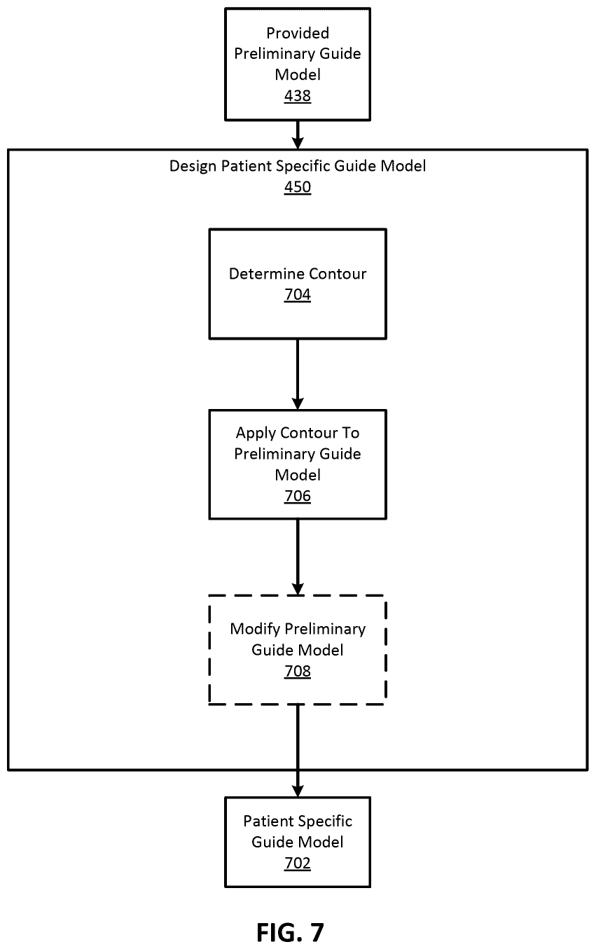

FIG. 7 illustrates an exemplary design module 450 configured to design a patient-specific guide model, according to one embodiment. The design module 450 may accept a preliminary guide model 438 and generate a patient-specific guide model 702. In one embodiment, the design module 450 includes a contour module 704, an application module 706, and/or an optional modification module 708.

Referring now to FIG. 7, the design module 450 may modify the preliminary guide model 438 such that the bone-facing and/or bone-contacting surfaces of the preliminary guide model 438 match a contour of the surfaces and/or joint of one or more bones where a step of an orthopedic procedure will be performed using the preliminary guide model 438.

The contour module 704 may determine a contour of the bones that will contact the preliminary guide model 438. The contour module 704 may use a bone model 404 and/or anatomic data 412 to determine the contour. For example, the contour module 704 may determine the shape of a dorsal surface of a calcaneus 222.

The application module 706 may apply the contour to the provided preliminary guide model 438 to custom contour a bone engagement surface of the preliminary guide model 438 to match the shape, contour, and/or one or more landmarks of a bone, such as a dorsal surface of a calcaneus 222. Applying the contour to the preliminary guide model 438 may convert the preliminary guide model 438 to a patient-specific guide model 702.

Generation of the contours of bone engagement surface of the preliminary guide model 438 may be performed in various CAD programs. In some embodiments, the shapes of the corresponding surface dorsal surface of a calcaneus 222 may be obtained directly from the bone model 404, anatomic data 412, CAD models and/or CT scan data, and simply copied onto the preliminary guide model 438. Various operations may be used to copy surfaces from one object to another. Additionally, or alternatively, various Boolean operations, such as a Boolean subtraction operation, may be used to remove material from a model for the body of the preliminary guide model 438 with a shape that matches the dorsal surface of a calcaneus 222.

In certain embodiments, the design module 450 may include an optional module, such as a modification module 708. The modification module 708 may enable a user such as a technician or surgeon to make additional modifications to the design and configuration of the preliminary guide model 438. In one embodiment, the user can change any of the features, trajectories, fixation holes, handle engagement holes, angles, configurations, or parameters of the preliminary guide model 438. For example, a surgeon may be aware of other concerns or anatomic aspects of a patient, for example on an opposite foot or in connection with a hip or other orthopedic joint which motivate the surgeon to adjust an angle of one of more trajectories of the preliminary guide model 438.

Alternatively, or in addition, a user may use the modification module 708 to modify instrumentation such as a guide. The user may add, remove, or modify steps and/or the instrumentation to create a patient-specific surgical procedure. In this manner, a user may configure features of a preliminary guide model 438 or modified preliminary guide model to a patient-specific osteotomy procedure the surgeon is planning for the patient.

The user may review the preliminary guide model 438 and make adjustments or revisions or make no adjustments or revisions. The output of the modification module 708 and/or the application module 706 is a patient-specific guide model 702.

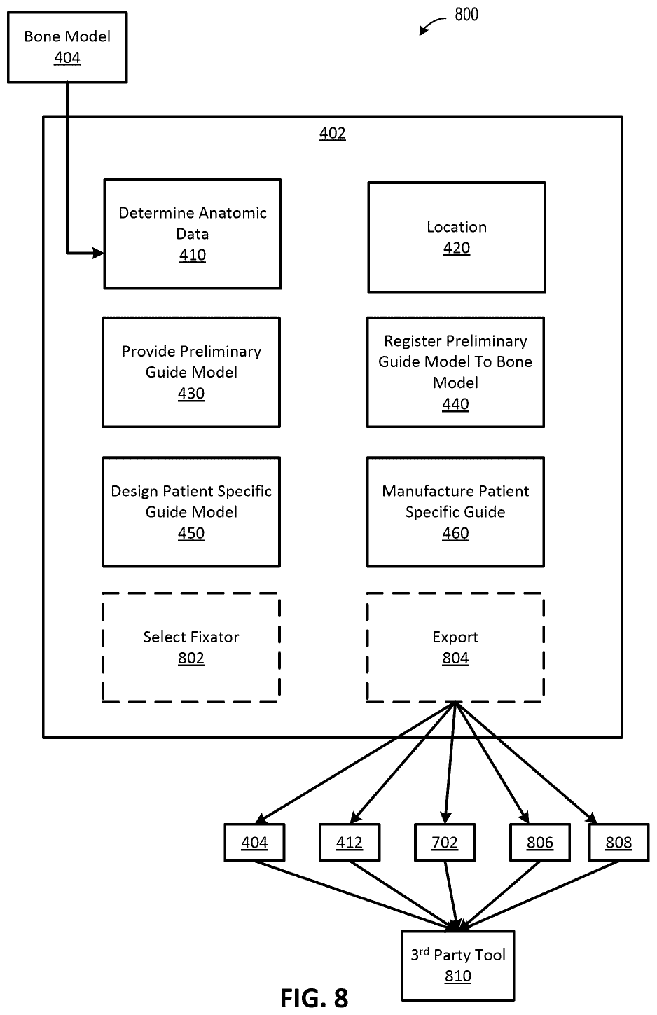

FIG. 8 illustrates an exemplary system 800 configured to generate one or more patient-specific instruments configured to correct a bone condition, according to one embodiment. The system 800 may include similar components or modules to those described in relation to FIG. 4. In addition, the system 800 may include a fixator selector 802 and/or an export module 804.

The fixator selector 802 enables a user to determine which fixator(s) to use for a surgical procedure planned for a patient. In one embodiment, the fixator selector 802 may recommend one or more fixators based on the bone model 404, the location, the trajectory, or input from a user or a history of prior surgical procedures performed. The fixator selector 802 may select a fixator model from a set of predefined fixator models or select a physical fixator from a set of fixators. The fixators may include a plate and associated accessories such as screws, anchors, and the like.

In one embodiment, the fixator selector 802 includes an artificial intelligence or machine learning module. The artificial intelligence or machine learning module is configured to implement one or more of a variety of artificial intelligence modules that may be trained for selecting fixator(s) based on anatomic data 412 and/or other input parameters. In one embodiment, the artificial intelligence or machine learning module may be trained using a large data set of anatomic data 412 for suitable fixator(s) identified and labeled in the dataset by professionals for use to treat a particular condition. The artificial intelligence or machine learning module may implement, or use, a neural network configured according to the training such that the artificial intelligence or machine learning module is able to select or recommend suitable fixator(s).

The export module 804 is configured to enable exporting of a patient-specific guide model 702 for a variety of purposes including, but not limited to, fabrication/manufacture of a patient-specific guide 406 and/or fixator(s), generation of a preoperative plan, generation of a physical bone model matching the bone model 404, and the like. In one embodiment, the export module 804 is configured to export the bone model 404, anatomic data 412, a patient-specific guide model 702, a preoperative plan 806, a fixator model 808, or the like. In this manner the custom instrumentation and/or procedural steps for a surgical procedure can be used in other tools. The preoperative plan 806 may include a set of step-by-step instructions or recommendation for a surgeon or other staff in performing a surgical procedure such as an osteotomy. The preoperative plan 806 may include images and text instructions and may include identification of instrumentation to be used for different steps of the surgical procedure. The instrumentation may include the patient-specific guide 406 and/or one or more fixators. In one embodiment, the export module 804 may provide a fixator model which can be used to fabricate a fixator for the surgical procedure.

The exports (404, 412, 702, 806, and 808) may be inputs for a variety of 3rd party tools 810 including a manufacturing tool, a simulation tool, a virtual reality tool, an augmented reality tool, an operative procedure simulation tool, a robotic assistance tool, and the like. A surgeon can then use these tools when performing a surgical procedure or for rehearsals and preparation for the surgical procedure. For example, a physical model of the bones, patient-specific guide 406, and/or fixators can be fabricated, and these can be used for a rehearsal operative procedure. Alternatively, a surgeon can use the bone model 404, preliminary guide model 438, and/or a fixator model to perform a simulated surgical procedure using an operative procedure simulation tool.

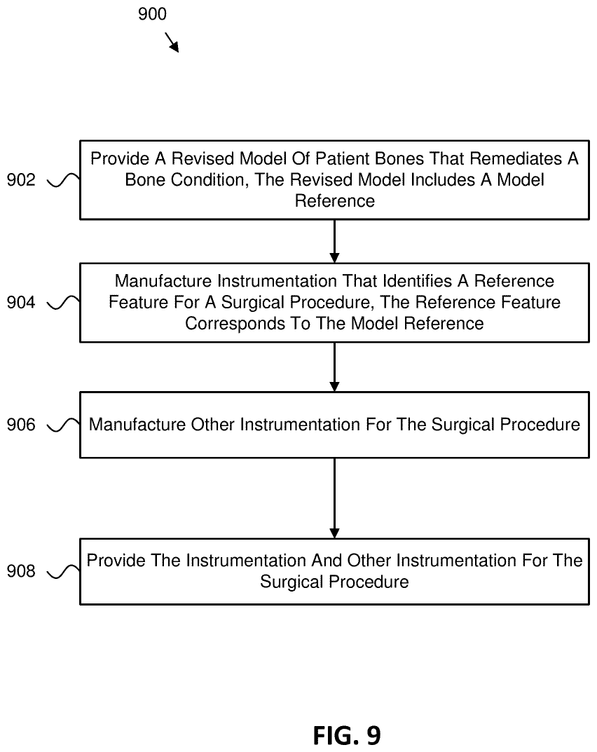

FIG. 9 is a flowchart of an example method 900. In some implementations, one or more method steps of FIG. 9 may be performed by a system 400, system 800, and/or user. The present disclosure leverages the use of models, such as computer models, and particularly models of a specific patient to provide and/or generate instrumentation, implants, and/or surgical plans that advanced patient care. Advantageously, these models are unique and customized for a particular patient. Thus, the models reflect the actual anatomical features and aspects of the patient.

One of the challenges with such solutions is how to translate, map, or convert from the model to the real world for a surgical procedure. Said another way, how to translate, map, or convert from one or more references (e.g., a model reference) within a model to one or more reference features on, in, or associated with, anatomical structures of the patient in the operating room or within the operating field. The present disclosure addresses this challenge by providing an apparatus, system, and method, that enables a surgeon to identify, create, form, and/or use a reference feature for a surgical procedure. The reference feature provides a reference and/or starting point on, in, or associated with anatomy of a patient such that steps, stages, features, or aspects planned and configured within the model can be accurately performed on, with, or to the anatomy of the patient. Method 900 provides one example of steps that can be used to implement changes, corrections, adjustments, or revisions setup or designed or engineered within the model on a patient's anatomy during a surgical procedure.

One aspect of the method 900 that assists in accomplishing the challenge, is by designing, creating, and/or engineering one or more model instruments within the model that use the one or more references (e.g., a model references). The one or more model instruments may use the one or more references to position and/or orient the one or more model instruments such that other steps of a surgical procedure can be performed in relation to those one or more model instruments and/or model references. Next, the method 900 may fabricate an actual instrument and/or implant that is an instantiation or a real world implementation of the model instrument. Advantageously, model references that interact with or are part of, or are integrated with the model instrument have this same relationship to the real world, actual, implementation of the model instrument fabricated according to the method 900.

For example, in one embodiment, pin locations for temporary fasteners, such as K-wires may be defined in the model. A model instrument is also defined within the model, that includes one or more holes that are sized just large enough to engage with and/or slide over the K-wires. The holes in the model instrument may be a model reference.

Alternatively, or in addition, the model instrument may include other features that are part of, or cooperate with a model reference. In one embodiment, the model instrument may include a feature such as a tissue engagement surface that is configured to engage with the tissue in a single position and/or orientation. In certain embodiments, the tissue engagement surface is a bone engagement surface. In another embodiment, the tissue engagement surface is a patient-specific bone engagement surface. Thus, in certain embodiments, holes in a model instrument and/or other features such as tissue engagement surface can serve as a model reference.

Next, the model instrument can be fabricated to form an actual instrument that includes the same features as the model reference (e.g., one or more holes that can indicate where pins are to be placed or one or more posts to engage holes or other features of a patient and/or a tissue engagement surface such as a bone engagement surface). In this manner, the instrument becomes a guide for a user in locating, identifying, determining, and/or forming reference features on, in, or associated with the anatomy of a patient.

For example, a surgeon can position and manipulate the actual instrument during a surgical procedure until the instrument contacts, seats, and/or registers to or against the tissue, such as one or more bone surfaces of a patient on a single bone and/or a plurality of bones. With the instrument in position (a position that corresponds to and matches the position of the model instrument in the model), a surgeon can mark, identify, form, or create the reference features that successfully map model references to reference features on a patient.

As shown in FIG. 9, method 900 may include providing a revised computer-aided design (CAD) model of one or more patient bones that remediates a bone condition, the revised CAD model having a model reference (block 902). For example, a system 400, system 800, and/or user may provide a revised computer-aided design (cad) model of one or more patient bones that remediates a bone condition, the revised cad model having a model reference. For example, where a patient has a bone deformity, a system 400, system 800, computer programmed device, and/or user can reposition one or more bones, resect portions of one or more bones, deploy implants, remove grafts, and/or make a variety of changes in the model until the model represents a correct condition for the bones of a patient.

As also shown in FIG. 9, method 900 may include manufacturing instrumentation that identifies a reference feature for a surgical procedure, the reference feature corresponding to the model reference (block 904). The reference feature may be a location for K-wires during a surgical procedure, a location for holes, a location for a bone tunnel, among other implementations of reference features. For example, a system 400, system 800, and/or user may manufacture instrumentation that identifies a reference feature for a surgical procedure, the reference feature corresponding to the model reference, as described above. In one embodiment, the instrumentation may be a positioner, also referred to as a positioning guide.

As further shown in FIG. 9, method 900 may include manufacturing other instrumentation for a surgical procedure (block 906). For example, a system 400, system 800, and/or user may manufacture other instrumentation for a surgical procedure, as described above. The other instrumentation may be any of the resection guides, fasteners, and/or complementary components, examples of which are described herein. In one embodiment, the other instrumentation is a plurality of resection guides. Each of the plurality of resection guides may include resection features (e.g., cut channels) that are designed and/or angled to remove different amounts of bone and/or to resect at different angles within three or more planes.

As also shown in FIG. 9, method 900 may include providing the instrumentation and other instrumentation for the surgical procedure (block 908). For example, a system 400, system 800, and/or user may provide the instrumentation and other instrumentation for the surgical procedure, as described above. The instrumentation and other instrumentation for the surgical procedure may be provided in a surgical kit, or tray and may be sterilized and/or packaged for use in a surgical procedure.

Although FIG. 9 shows example blocks or steps of a process or method 900, in some implementations, a method 900 may include additional steps, fewer steps, different steps, or differently arranged steps than those depicted in FIG. 9. Additionally, or alternatively, two or more of the steps of a method 900 may be performed in parallel.

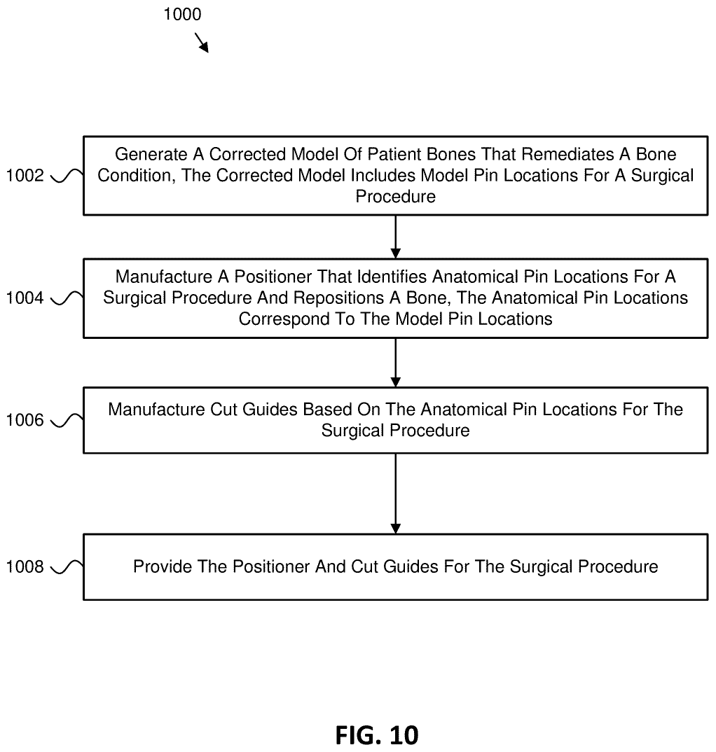

FIG. 10 is a flowchart of an example method 1000. In some implementations, one or more method steps of FIG. 10 may be performed by a system 400, system 800, and/or user.

As shown in FIG. 10, method 1000 may include generating a corrected model of one or more patient bones, the corrected model includes model pin location for a surgical procedure (block 1002). For example, a system 400, system 800, and/or user may generate a corrected model of one or more patient bones, the corrected model includes model pin location for a surgical procedure, as described above. Model pin locations and/or the corresponding pins may serve as one example of a model reference, as used in method 900.

As also shown in FIG. 10, method 1000 may include manufacturing a positioner (aka positioning guide) that identifies anatomical pin locations for a surgical procedure and/or repositions a bone. The anatomical pin locations may correspond to the model pin location (block 1004). For example, a system 400, system 800, and/or user may manufacture a positioner (aka positioning guide) that identifies anatomical pin locations for a surgical procedure and/or repositions a bone, the anatomical pin locations corresponding to the model pin location, as described above. Anatomical pin locations and/or the corresponding pins may serve as one example of a reference feature, as used for example in method 900. In one embodiment, the positioner may be made by additive manufacturing. Furthermore, the positioner may be made from a material transparent to medical imaging such as fluoroscopy (e.g., a polymer such as Nylon 12). Alternatively, or in addition, the positioner may be made from a material that is radiolucent to medical imaging such as fluoroscopy (e.g., a polymer such as Nylon 12).

As further shown in FIG. 10, method 1000 may include manufacturing one or more cut guides and/or one or more resection guides based on the anatomical pin locations for a surgical procedure (block 1006). For example, a system 400, system 800, and/or user may manufacture cut guides or resection guides based on the anatomical pin locations for a surgical procedure, as described above. The resection guides may include one or more holes that are configured to engage or accept the pins and slide down over and/or slide up off of the pins during a surgical procedure. In one embodiment, the one or more resection guides may be made by additive manufacturing. Furthermore, the one or more resection guides may be made from a material transparent to medical imaging such as fluoroscopy (e.g., a polymer such as Nylon 12). With the positioner and/or resection guides transparent to medical imaging a surgeon can intraoperatively check medical imaging and confirm that the planned resection will result in a desired level of correction. Alternatively, or in addition, the positioner and/or resection guide may include one or more alignment guides that can be seen in medical imaging such that a surgeon can use these guides to confirm the planned correction will result in a desired level of correction.

As also shown in FIG. 10, method 1000 may include providing the positioner (aka positioning guide) and/or cut guides or resection guides for the surgical procedure (block 1008). For example, a system 400, system 800, and/or user may provide the positioner (aka positioning guide) and cut guides and/or resection guides for the surgical procedure, as described above. The positioner cut guides and/or resection guides for the surgical procedure may be provided in a surgical kit, or tray and may be sterilized and/or packaged for use in a surgical procedure.

Although FIG. 10 shows example blocks or steps of a process or method 1000, in some implementations, a method 1000 may include additional steps, fewer steps, different steps, or differently arranged steps than those depicted in FIG. 10. Additionally, or alternatively, two or more of the steps of a method 1000 may be performed in parallel.

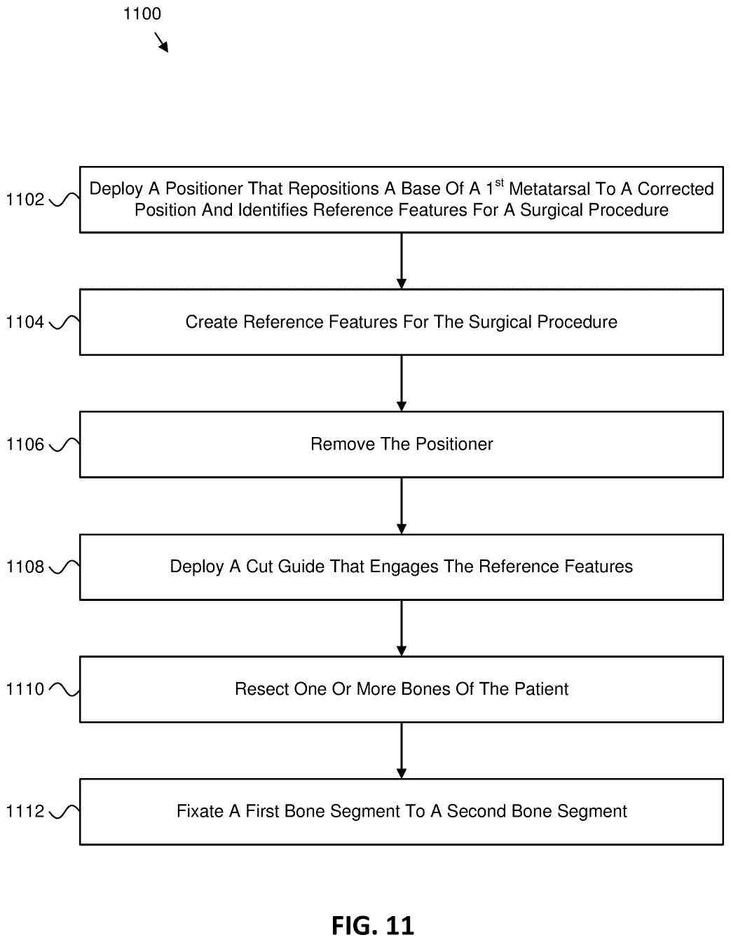

FIG. 11 is a flowchart of an example method 1100 for performing a surgical procedure. In particular, the method 1100 can be used to perform a Lapidus arthrodesis. In some implementations, one or more method steps of FIG. 11 may be performed by a system 400, system 800, medical robot, and/or user.

As shown in FIG. 11, method 1100 may include deploying a positioner that repositions a base of a first metatarsal of a patient to a corrected position and identifies one or more reference features for a surgical procedure (block 1102). In one embodiment, a patient may present with a first metatarsal 208 in a deformed position. After forming an incision in the skin and soft tissue on a medial side of a TMT joint, a surgeon may place the positioner against a cortical surface of the base of the first metatarsal 208 and/or against a cortical surface of a medial cuneiform 202. In certain embodiments, a portion of the positioner may extend at least in part into a space of the TMT joint between the medial cuneiform 202 and the first metatarsal 208.

Optionally, or in certain embodiments, a surgeon may “loosen” the joint or “free” the joint by resecting or deburring or otherwise removing parts of connective tissue between the medial cuneiform 202 and the first metatarsal 208 of the TMT joint. Those of skill in the art will appreciate that this process of loosening or freeing a joint may include incisions, resection, and/or dissection directed towards releasing soft tissue such that a bone or one or more bones of a joint can be translated and/or rotated (e.g., repositioned or moved). Accordingly, in certain embodiments and/or surgical procedures this step may be referred to as a release or releasing the joint or releasing a metatarsal or a cuneiform.

In one example, a system 400, system 800, and/or user may deploy a positioner that repositions a base of a first metatarsal of a patient to a corrected position and identifies one or more reference features for a surgical procedure, as described above. Advantageously, positioning the positioner in the position defined in the model enables a surgeon to identify where reference features should be on the anatomy of the patient.

As also shown in FIG. 11, method 1100 may include creating one or more reference features for a surgical procedure (block 1104). For example, a system 400, system 800, and/or user may create one or more reference features for a surgical procedure. In one embodiment, the positioner may include holes that identify where openings can be formed in one or more bones and/or where pins can be deployed. The holes in the bone(s) and/or the pins deployed in the bone(s) can serve as the reference feature(s) for subsequent steps in a surgical procedure.

In one embodiment, the positioner includes holes for pins that a surgeon deploys through the holes in the positioner. With the pins deployed, a surgeon is assured that reference features on the anatomy match the model references that were used to preplan the surgical procedure. In another embodiment, a surgeon may drill holes in one or more bone(s) using holes in the positioner as a guide. The holes in the bone(s) may serve as reference features and may be configured to accept projections in one or more other instruments and/or implants used in the surgical procedure.

As further shown in FIG. 11, method 1100 may include removing the positioner (block 1106). For example, a system 400, system 800, and/or user may remove the positioner. In one embodiment, since the positioner has accomplished its purpose for the surgical procedure, the positioner may be removed leaving just one or more reference features (e.g., pins, holes, anchors, or the like).

As also shown in FIG. 11, method 1100 may include deploying a cut guide or resection guide that engages the one or more reference features (block 1108). For example, a system 400, system 800, and/or user may deploy a cut guide or resection guide that engages the one or more reference features. In embodiments where the reference feature is one or more pins extending from one or more bones, the resection guide may slide down over the pins and contact the bone(s). The resection guides can include resection features that guide a surgeon in making resections of one or more bones of the patient for the surgical procedure.

As further shown in FIG. 11, method 1100 may include resecting one or more bones of the patient (block 1110). For example, a system 400, system 800, and/or user may resect one or more bones of the patient. For example, a surgeon may use a cutting tool to resect bone or bone sections aligned with resection features of the deployed resection guides.

As also shown in FIG. 11, method 1100 may include fixating a first bone segment to a second bone segment (block 1112). For example, a system 400, system 800, and/or user may fixate a first bone segment to a second bone segment. Those of skill in the art will appreciate that the surgeon may use a variety of different types of fasteners to complete the fixation step and these fasteners may be permanent or temporary.

Although FIG. 11 shows example blocks or steps of a process or method 1100, in some implementations, a method 1100 may include additional steps, fewer steps, different steps, or differently arranged steps than those depicted in FIG. 11. Additionally, or alternatively, two or more of the steps of a method 1100 may be performed in parallel. Alternatively, or in addition, certain steps of method 1100 may be the same as corresponding steps of method 1000.

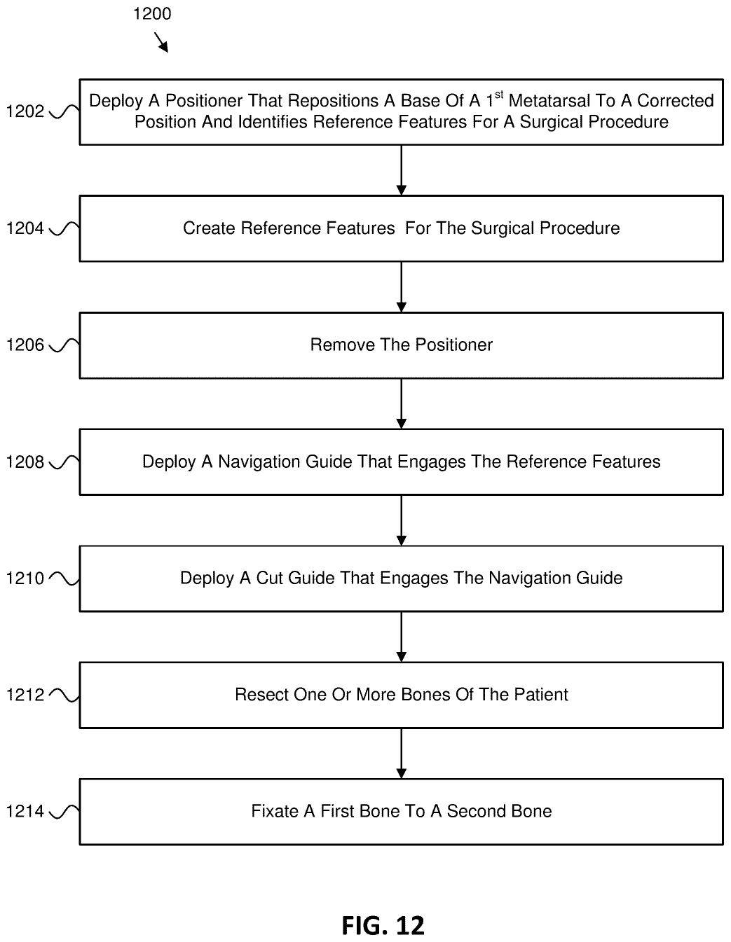

FIG. 12 is a flowchart of an example method 1200. In some implementations, one or more method steps of FIG. 12 may be performed by a system 400, system 800, and/or user.

As shown in FIG. 12, method 1200 may include deploying a positioner that repositions a base of a first metatarsal of a patient to a corrected position and identifies one or more reference features for a surgical procedure (block 1202). For example, a system 400, system 800, and/or user may deploy a positioner that repositions a base of a first metatarsal of a patient to a corrected position and identifies one or more reference features for a surgical procedure. As also shown in FIG. 12, method 1200 may include creating one or more reference features for a surgical procedure (block 1204). For example, a system 400, system 800, and/or user may create one or more reference features for a surgical procedure. As further shown in FIG. 12, method 1200 may include removing the positioner (block 1206). For example, a system 400, system 800, and/or user may remove the positioner.

As also shown in FIG. 12, method 1200 may include deploying a navigation guide that engages the one or more reference features (block 1208). In contrast to method 1100, method 1200 may use a navigation guide together with one or more resection guides. The resection guides may engage and/or couple with the navigation guide. For example, a system 400, system 800, and/or user may deploy a navigation guide that engages the one or more reference features. Navigation guides may include the examples and/or embodiments described in U.S. Provisional Patent Application Ser. No. 63/482,038, entitled “APPARATUS, SYSTEM, AND METHOD FOR OSTEOTOMIES” filed Jan. 28, 2023, and U.S. patent application Ser. No. 18/422,933, entitled “APPARATUS, SYSTEM, AND METHOD FOR OSTEOTOMIES” filed Jan. 25, 2024, which are incorporated herein by reference in their entirety and for all purposes.

As further shown in FIG. 12, method 1200 may include deploying a cut guide that engages the navigation guide (block 1210). For example, a system 400, system 800, and/or user may deploy a cut guide that engages the navigation guide. As also shown in FIG. 12, method 1200 may include resecting one or more bones of the patient (block 1212). For example, a system 400, system 800, and/or user may resect one or more bones of the patient. As further shown in FIG. 12, method 1200 may include fixating a first bone to a second bone (block 1214). For example, a system 400, system 800, and/or user may fixate a first bone to a second bone. Alternatively, or in addition, method 1200 may include fixating a first bone fragment to a second bone fragment.

Although FIG. 12 shows example blocks or steps of a process or method 1200, in some implementations, a method 1200 may include additional steps, fewer steps, different steps, or differently arranged steps than those depicted in FIG. 12. Additionally, or alternatively, two or more of the steps of a method 1200 may be performed in parallel.

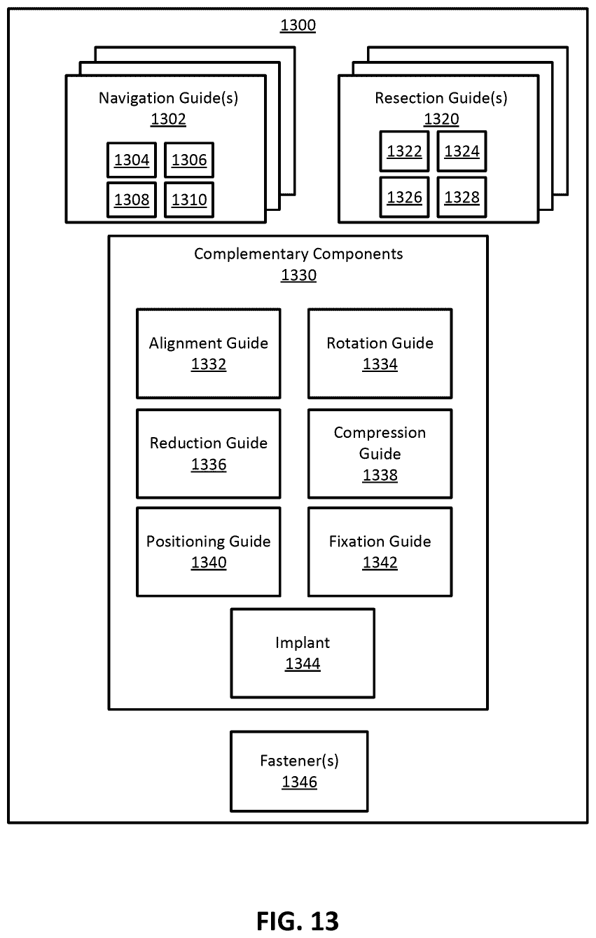

FIG. 13 illustrates an exemplary system 1300, according to one embodiment. The system 1300 may include one or more navigation guides 1302, one or more resection guides 1320, one or more complementary components 1330, and one or more fasteners 1346. While a system 1300 can be used for a variety of procedures, one or more features, components, and/or aspects of the system 1300 may be particularly suited for one or more osteotomies on one or more bones of a patient's anatomy such as a patient's foot, ankle, wrist, hand, shoulder, or the like. In certain embodiments, the system 1300 may not include a navigation guide 1302.

A navigation guide 1302 can serve to guide a surgeon during one or more stages of a surgical procedure. In one embodiment, the navigation guide 1302 can provide a guide or a template for a surgeon for where to form, provide, position, and/or orient one or more reference features.

In the present disclosure, a reference feature(s) (also referred to as anatomical references) can provide an intraoperative feature that can enable a surgeon to model patient anatomy, model instrumentation, model implants, and/or practice a surgical procedure either virtually or using physical models, and then during the surgery configure, position, orient and/or register instrumentation, implants, and/or other surgical components to perform the surgery using the registered/positioned instrumentation, implants, and/or other surgical components developed, designed, and/or refined in a virtual or simulated procedure. In one example embodiment, a reference feature can be realized by a hole, tunnel, or other opening, in a bone or other hard tissue and/or in soft tissue. In another example embodiment, a reference feature can be realized by a hole, tunnel, or other opening, in a bone or other hard tissue and/or in soft tissue together with another structure such as a fastener such as a bone screw, a K-wire, or the like.

Advantageously, the navigation guide 1302 facilitates providing, forming, establishing, and/or configuring one or more reference features for a surgical procedure. The navigation guide 1302 can include a body 1304, an opening 1306, and one or more position indicators 1308. In certain embodiments, a navigation guide 1302 may include a bone attachment feature 1310. In such embodiments, the bone attachment feature 1310 can be used to secure the navigation guide 1302 to a bone or bone fragment, at least temporarily.

The one or more resection guides 1320 assist a surgeon in performing one or more different resection steps for an osteotomy procedure. In certain embodiments, a resection guide 1320 includes one or more resection features 1322 and one or more bone attachment features 1324. The resection features 1322 can take a variety of forms and/or embodiments. Similarly, the bone attachment features 1324 can take a variety of forms and/or embodiments. The bone attachment features 1324 may be similar to, the same as, or different from, the bone attachment feature 1310 that can be used with the navigation guide 1302. In one embodiment, a resection guide 1320 may include a bone attachment feature 1324 that is at an oblique angle relative to one or more reference features. The obliquely angled bone attachment feature 1324 may provide more stability for the resection guide 1320 during a resection.

The resection features 1322 provide a guide for a surgeon using a cutting tool to resect a bone, one or more bones, or other tissues of a patient. The bone attachment features 1324 serve to secure the resection guide 1320 to one or more bones, one or more bone fragments, and/or one or more other structures. In one example, a bone attachment feature 1324 can include a hole in the resection guide 1320 together with a temporary fastener such as a K-wire or pin.

The bone attachment features 1324 can be used to engage or connect or attach a resection guide 1320 to one or more bones, or bone fragments, of a patient. The bone attachment features 1324 may include any of a wide variety of fasteners including, but not limited to, holes, prongs, spikes, fastening devices, and/or the like. Effective engagement or connection of the resection guide 1320 to one or more bones along a single bone, across a single joint, across a plurality of joints, or the like, can ensure that cut surfaces are formed in desired locations and orientations and mitigate removal of hard tissue and/or soft tissue in undesired locations.

In certain embodiments, a resection guide 1320 may include one or more bone engagement surfaces 1326 and/or one or more landmark registration features 1328. In certain embodiments, a landmark registration feature 1328 may extend from one or more sides of the resection guide 1320 and engage with one or more landmarks of a bone of a patient. Registration of the landmark registration feature 1328 to the landmark of the bone can serve to confirm that a surgeon has located a desired placement and/or orientation for a resection guide 1320. In certain embodiments, the navigation guide 1302 can include one or more bone engagement surfaces 1326 on a surface of the navigation guide 1302 that faces a bone or bone fragment. In other embodiments, a resection guide 1320 may include no bone engagement surface 1326 or landmark registration feature 1328.

In certain embodiments, the bone engagement surfaces 1326 are patient-specific: contoured to match a surface of one or more bones the resection guide 1320 contacts during the procedure. Alternatively, or in addition, the bone engagement surface 1326 may not be patient-specific and may, or may not, contact a bone surface during use of the resection guide 1320. Those of skill in the art appreciate that one or more sides of any of the members of the system 1300 may include one or more bone engagement surfaces 1326. Consequently, one or more sides of the navigation guide 1302, the resection guide(s) 1320, the complementary components 1330, fasteners 1346, and/or the implants 1396 may include one or more bone engagement surfaces 1326.

In certain embodiments, other members of the system 1300 may be configured such that a bone engagement surface 1326 of a resection guide 1320 may not be needed. Instead, a positioning guide 1340 may include a bone engagement surface 1326. Further the positioning guide 1340 may be made from a less expensive material, such as a polymer and may include a bone engagement surface 1326 and the resection guide 1320 may not be patient-specific. Instead, the resection guide 1320 may be made from a more durable material such as metal. In such an embodiment, the positioning guide 1340 may be disposable and may be patient-specific which the resection guide 1320 may be reusable and may be useable with a number of different patients. In one embodiment, the resection guide 1320 may be patient-matched.

Alternatively, or in addition, the resection guide 1320 may be selected from a kit, collection, or repository of a number of resection guides 1320: each having a different configuration for resecting one or more parts of one or more bones. For example, each member of the repository/kit may include a one or more numbers. A first number may indicate a number of millimeters a specific resection feature of the resection guide 1320 will remove and a second number may indicate a number of millimeters another resection feature of the resection guide 1320 will remove. For example, a resection guide 1320 may include a +1 near a proximal end and a +1 near a distal end of the resection guide 1320 (indicated +1:+1) and may include a proximal end resection feature and a distal resection feature. In such an example, the resection guide 1320 will remove 1 millimeter from distal end of a medial cuneiform 202 and 1 millimeter from proximal end of a first metatarsal 208. Accordingly, a kit may include resection guide 1320 with configurations such as +2:+1, or +1:+3, or the like. In certain embodiments, the kit may include resection guide 1320 configurations such as +0:+1, or 0:+3 where the 0 indicates that the resection will be done according to a preoperative plan developed using the model and the +number indicates an additional amount of tissue to be resected beyond the amount planned for in the preoperative plan.

Having a plurality of different resection guides 1320 can be advantageous where a surgeon sees something intraoperatively that causes them to decide to change the preoperative plan. Often a surgeon seeks to minimize the amount of tissue removed during a surgical procedure and to create a resected bone surface that will support an effective union and the preoperative plan also works towards these goals. However, during the operation a surgeon may alter the plan based on what they encounter and thus may opt to use a different resection guide 1320 than was originally planned. Advantageously, the present disclosure facilitates this.

Alternatively, or in addition, a resection guide 1320 may be configured to resection one or the other of the bones of a joint.

different number positioning angle (repositioning or correction angle), the angles may differ by two or five degrees for example. In such an embodiment, each positioning guide 1340 may not be patient-specific to a particular patient but may provide the desired amount of positioning to meet the goals of the surgeon. In certain embodiments, a preoperative plan generated based on the present disclosure may include a recommendation for the positioning guide 1340 to be used, even if the recommended positioning guide 1340 is not patient-specific to the particular patient.

The complementary components 1330 can serve to assist a surgeon during one or more steps of a procedure. Those of skill in the art appreciate that a number of components can serve as complementary components 1330. One or more of the features, functions, or aspects of the complementary components 1330 can include patient-specific features.

Examples of complementary components 1330 include, but are not limited to, an alignment guide 1332, a rotation guide 1334, a reduction guide 1336, a compressor 1338, a positioning guide 1340, a fixation guide 1342, and/or one or more implants 1344. In general, the complementary components 1330 serve to assist a surgeon in performing the function included in the name of the complementary component 1330. Thus, an alignment guide 1332 can help a surgeon align bones, parts of bones, anatomical body parts, or other parts of a patient as part of a procedure. A rotation guide 1334 can help a surgeon rotate one or more bones, parts of bones, or other body parts of a patient as part of a procedure.

A reduction guide 1336 can help a surgeon position and/or orient one or more bones, bone fragments, or other parts of a patient as part of a procedure in order to reduce the bone, bones, bone fragments, or other parts and/or in order to position and/or orient the bone, bones, bone fragments, or other parts to a desired position and/or orientation. A compressor 1338 can help a surgeon compress one or more bones, bone fragments, or other parts of a patient together or against an implant as part of a procedure. A positioning guide 1340 can help a surgeon position one or more bones, parts of bones, other parts of a patient, instruments, or other structures as part of a procedure. One example of a positioning guide 1340 is a positioner, described above.

In certain embodiments, the positioning guide 1340 may be designed and fabricated to be patient-specific. The patient-specific aspects can include a patient-specific bone engagement surface, a predefined angle for reorienting one or more bone or bone parts within one or more planes, a predefined position for bone attachment features 1324 or fasteners 1346, or the like. Alternatively, or in addition, the positioning guide 1340 may be selected from a kit, collection, or repository of a number of positioning guides 1340: each having a different configuration for one or more aspects/attributes of the positioning guide 1340. For example, each member of the repository/kit may include a different positioning angle (repositioning or correction angle), the angles may differ by two or five degrees for example. In such an embodiment, each positioning guide 1340 may not be patient-specific to a particular patient but may provide the desired amount of positioning to meet the goals of the surgeon. In certain embodiments, a preoperative plan generated based on the present disclosure may include a recommendation for the positioning guide 1340 to be used, even if the recommended positioning guide 1340 is not patient-specific to the particular patient.

A fixation guide 1342 can help a surgeon in completing one or more temporary or permanent fixation steps for one or more bones, parts of bones, or other parts of a patient as part of a procedure. The fixation guide 1342 may include and/or may use one or more components of a fastener or fixation system including implant hardware of the fastener or fixation system.

One example of a complementary components 1330 may include a compressor/distractor, which is one example of a compressor 1338. The compressor/distractor can be used to compress and/or distract bones or parts of bones involved in a procedure.

Advantageously, the system 1300 can help a surgeon overcome one or more of the challenges in performing an osteotomy procedure, particularly on bones of a hand or of a foot of a patient, such as on the forefoot, midfoot, or hindfoot. One challenge during an osteotomy procedure can be maintaining control of, and/or position, and/or orientation of a bone, one or more bones, and/or bone pieces/fragments, particularly once a resection or dissection is performed. Advantageously, the navigation guide 1302, resection guide(s) 1320, and/or complementary components 1330 can be configured to assist in overcoming this challenge.

Advantageously, the system 1300 can help a surgeon in positioning, placing, and/or orienting a instruments for the procedure accurately. Modern techniques may include preoperative planning, simulation, or even practice using computer models, 3D printed models, virtual reality systems, augmented reality systems or the like. However, simulations and models are still different from actually positioning a one or more instruments on a patient's bone, joint, or body part during the procedure. The system 1300 can include a number of features, including for example the reference features, patient-specific features (which can include reference features), to assist the surgeon with the positioning.

Advantageously, the system 1300 can help a surgeon in securing guides of the osteotomy system 1300, as well as how to readily remove the instrumentation without disturbing a reduction, shifting, reorienting, or repositioning one or more bones or parts of bones while removing the guide. In certain embodiments, the system 1300 is configured to permit removal of instrumentation while keeping temporary fasteners in place for use in subsequent steps of an osteotomy procedure. Alternatively, or in addition, the system 1300 facilitates positioning of temporary or permanent fasteners during one step of the osteotomy procedure for use in a subsequent step of the osteotomy procedure. For example, holes or openings formed in the bone during one step of the osteotomy procedure can serve as pilot or starter holes for subsequent permanent fasteners and/or other hardware. Removal of instrumentation during an osteotomy procedure can be particularly challenging where translation and/or rotation of the bones involved in the osteotomy procedure is required for the success of the osteotomy procedure. Advantageously, the system 1300 accommodates translation and/or rotation of the bones during the osteotomy procedure while facilitating a successful outcome for the osteotomy procedure.

Advantageously, the components of the system 1300 can be specifically designed for a particular patient. Alternatively, or in addition, the components of the system 1300 can be specifically designed for a class of patients (e.g., patient-matched). Each of the components of the system 1300 can be designed, adapted, engineered and/or manufactured such that each feature, attribute, or aspect of the component is specifically designed to address one or more specific indications present in a patient. Advantageously, cuts made for the osteotomy procedure can be of a size, position, orientation, and/or angle that provides from an optimal osteotomy and/or outcome with minimal risk of undesirable resection. In one embodiment, the components of the system 1300 can be configured such that an osteotomy is performed that enables a correction in more than one plane in relation to the parts of the body of the patient. For example, cut channels a resection guide 1320 can be oriented and configured such that when the bones are fused/fixated the correction results from translation, rotation, and/or movement of bones or bone parts in two or more planes (e.g., sagittal and transverse).

In certain embodiments, the one or more fasteners 1346 can include both, one or more permanent fasteners and one or more temporary fasteners. Typically, the fasteners 1346 may be used during a variety of different steps of a procedure. Temporary fasteners are often used because they can securely hold bone or bone fragments while steps of the procedure are conducted. A common temporary fastener that can be used with system 1300 is a K-wire, also referred to as a pin.

In certain embodiments, the exemplary system 1300 may include a plurality of navigation guides 1302, resection guides 1320, complementary components 1330, and/or fasteners 1346. For example, a surgeon may plan to resect a plurality of wedge segments from one or more bone(s) in order to accomplish a desired correction. One or more wedge segments may be resected from a medial side of a patient's foot and another one or more wedge segments may be resected from a lateral side of the patient's foot. These wedge segments may extend part way into the foot, or through from one side of the foot to the other. Of course, multiple wedge segments may be formed on one side of the foot as well.

In certain embodiments, the components of the system 1300 may be made as small as possible to minimize the amount of soft tissue that is opened or disturbed in the patient for the osteotomy procedure. Alternatively, or in addition, walls and/or sides of the components may be beveled and/or angled to avoid contact with other hard tissue or soft tissues in the operating field for the osteotomy procedure and/or to facilitate handlings and positioning by a user.

Those of skill in the art will appreciate that for certain osteotomy procedures a particular complementary component 1330 may not be needed or a particular complementary component 1330 may be optional for use in the osteotomy procedure. Similarly, those of skill in the art will appreciate that certain features of the navigation guide 1302, resection guides 1320, complementary components 1330, fasteners 1346 can be combined into one or more of apparatus or devices or may be provided using a plurality of separate devices.

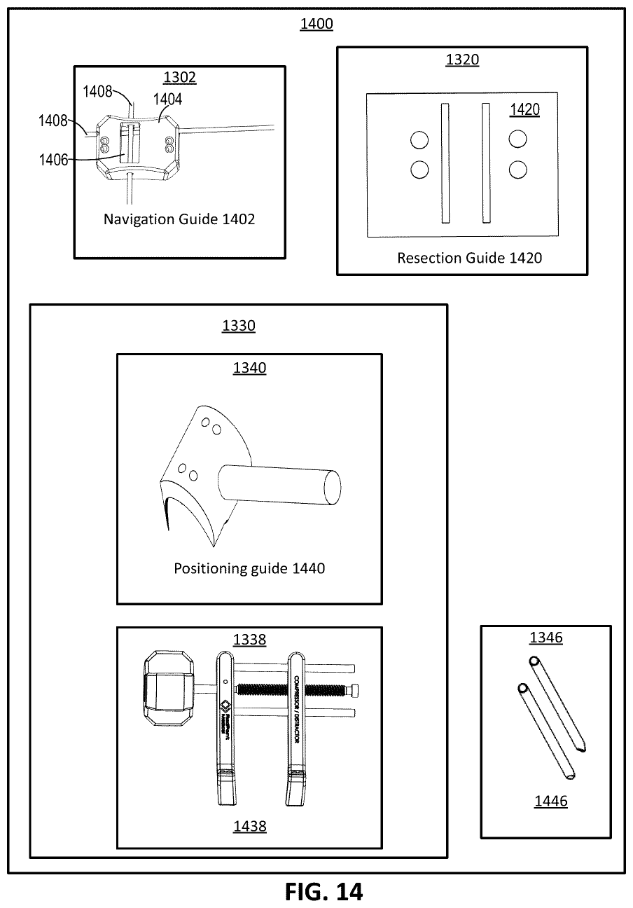

FIG. 14 illustrates an exemplary osteotomy system 1400, according to one embodiment. The osteotomy system 1400 may include a navigation guide 1302, one example of which is navigation guide 1402, one or more resection guides 1320, one example of which is resection guide 1420, one or more other complementary components 1330, such as positioning guide 1340, one example of which is positioning guide 1440 and/or a compressor 1338, one example of which is compressor/distractor 1438, and one or more fasteners 1346, such as fasteners 1446. The osteotomy system 1400 can be used for a variety of surgical procedures.

In certain embodiments, the positioning guide 1440 can be used to create, form, establish, or provide one or more reference features or anatomical references. In particular, the reference features may be formed, created, established, or provided within a single bone, one or more bones of a joint, one or more bones of a plurality of joints, or the like. Initially, the positioning guide 1440 may be positioned before reference features are formed, created, established, or provided. In one embodiment, after using the positioning guide 1440 a surgeon may use the navigation guide 1402 to validate and/or confirm the positioning and/or trajectory of reference features before proceeding with a surgical procedure.

In one embodiment, a user may position the navigation guide 1402 before reference features are formed, created, established, or provided. Alternatively, or in addition, the navigation guide 1402 can be used to form, create, establish, or provide the reference features. In one embodiment, a user, such as a surgeon may initially position the navigation guide 1402 in an approximate position for where the reference features are to be established. Next, the surgeon may use medical imaging, such as fluoroscopy, to determine where the navigation guide 1402 is in relation to one or more bones of a patient, and/or in relation to one or more bones and one or more joints of a patient. In the illustrated embodiment, the position indicators 1408 can be made of a material that is visible under fluoroscopy. One example material is metal.

Advantageously, the navigation guide 1402 can include two position indicators 1408 that overlap to form a crosshair within an opening of the navigation guide 1402. A surgeon can visually compare a position of the crosshair to one or more anatomical structures and/or reference features of a patient.

In one example embodiment, a surgeon may position the crosshair relative to a joint, a plurality of joints, one or more bones, or another reference feature of the patient to form, create, establish, or provide one or more reference features for a surgical procedure. By comparing the crosshair to an initial approximate position, a surgeon can determine if the initial placement of the navigation guide 1402 is in a desired position. If the initial placement, is not in the desired position, a surgeon may adjust the position of the navigation guide 1402 and check the crosshair again visually or with the help of medical imaging, such as fluoroscopy, to determine if the navigation guide 1402 is now in a desired position. If not, this process of repositioning and rechecking can be repeated until the surgeon positions one or more of the crosshair, a first position indicator 1408, and/or a second position indicator 1408 in a desired position for forming, creating, establishing, or providing one or more reference features.

In the illustrated embodiment, the positioning guide 1440 may include one or more position indicators. (See for example FIG. 19) Position indicators serve to indicate a position of one or more objects in relation to another one or more objects. In certain embodiments, two position indicators work may together to indicate position information about one or more objects.

In certain embodiments, the exemplary osteotomy system 1400 may include both a navigation guide 1402 and a positioning guide 1440. In such an embodiment, a surgeon may choose to use both the navigation guide 1402 and the positioning guide 1440 or just one of the navigation guide 1402 or the positioning guide 1440. Those of skill in the art will appreciate that either the navigation guide 1402 or the positioning guide 1440 of both may be used for forming, creating, establishing, or providing one or more reference features. In another embodiment, the exemplary osteotomy system 1400 may include just a positioning guide 1440 and no navigation guide 1402.

The osteotomy system 1400 includes resection guide 1420. The resection guide 1420 facilitates resection of hard tissue and/or soft tissue of a patient for a surgical procedure. In one embodiment, the resection guide 1420 can be a standalone, separate apparatus. In another embodiment, the resection guide 1420 can be an apparatus that couples to, integrates with, and/or cooperates with a navigation guide 1402 to assist a surgeon in resection of patient tissue.

The osteotomy system 1400 may include a plurality of complementary components 1330. The osteotomy system 1400 includes positioning guide 1440 that facilitates positioning one bone or bone fragment in relation to another bone, a bone fragment, another joint, a plurality of joints, or another reference feature or anatomical feature. In certain embodiments, the positioning guide 1440 can serve to provide, guide, form, create, establish, or provide one or more reference features. Alternatively, or in addition, the positioning guide 1440 can serve to position one or more bones for a surgical procedure. For example, in one embodiment, the positioning guide 1440 can be used to position a metatarsal for a Lapidus arthrodesis surgical procedure.

In certain embodiments, the positioning guide 1440 may include one or more position indicators 1408. In the illustrated embodiment, the positioning guide 1440 may include one or more position indicators. (See for example FIG. 19) Position indicators serve to indicate a position of one or more objects in relation to another one or more objects. In certain embodiments, two position indicators work may together to indicate position information about one or more objects.

In the illustrated embodiment, the position indicators may be used to show the position of a 1^{st} metatarsal relative to 2^{nd} metatarsal before a corrective osteotomy is completed. Of course, one or more position indicators may be used the position of a 1^{st} metatarsal relative to 2^{nd} metatarsal after the 1^{st} metatarsal is repositioned to a corrected position (e.g., predetermined position).

The osteotomy system 1400 may include a compressor/distractor 1438 that facilitates compressing one bone or bone fragment toward or to contact another bone, a bone fragment, a joint, a plurality of joints, or another reference feature or anatomical feature. In certain embodiments, the compressor/distractor 1438 can also be used to distract one bone or bone fragment away from another bone, a bone fragment, joint, a plurality of joints, or another reference feature or anatomical feature.

In certain embodiments, the one or more fasteners 1446 can include one or more permanent fasteners and/or one or more temporary fasteners. Typically, the fasteners 1446 may be used during a variety of different steps of a procedure. Temporary fasteners are often used because they can securely hold bone or parts/fragments of bones while steps of the procedure are conducted. A common temporary fastener that can be used with osteotomy system 1400 is a K-wire, also referred to as a pin or guide pin.

The osteotomy system 1400 for remediating a bone condition present in a patient includes a positioning guide 1440. The positioning guide 1440 includes a body 1448 that includes a proximal side 1450, a distal side 1452, a medial side 1454, a lateral side 1456, an inferior side 1458, and a superior side 1460.

The positioning guide 1440 also includes at least one opening 1462 that extends from one side of the body 1448 to an opposite side of the body 1448. The opening(s) 1462 are one example embodiment a reference feature guide or reference for creating a reference feature on patient anatomy. The reference feature guide serves to guide the creation, deployment and/or formation of a reference feature in one bone, another bone or both one bone and another bone. In one embodiment, a reference feature guide can be used to form a single reference feature. In another embodiment, a single reference feature guide can be used to form a plurality of reference feature. In another embodiment, a plurality of reference feature guides can be used to form a plurality of reference features. A reference feature guide may extend through the body such as with an opening 1462 alternatively, or in addition, a reference feature guide may extend from or connect or couple to the body 1448 such as for example with a spike or prong that extends from the body 1448.

In one embodiment, the opening 1462 is an anatomical pin location for a surgical procedure that corresponds to a model pin location in a model. In certain embodiments, one or more openings 1462 may indicate pin locations for a reference feature and one or more of the same openings 1462 and/or one or more other openings 1462 may enable an alignment or other repositioning of a bone fragment as part of a surgical procedure. For example, certain openings 1462 may be positioned more distally or in another position such that pins in bone at these openings 1462 can be used to rotate, reposition, align, compress, and/or realign one or more bones of a patient. In the illustrated embodiment, the openings 1462 have a round cross-section.

The system 1400 also includes one or more instruments configured to participate in addressing the bone condition present in the patient. Examples of the instruments include, but are not limited to, a one or more resection guides 1420, a positioning guide 1440, a compressor/distractor 1438, a fastener 1446, or the like.

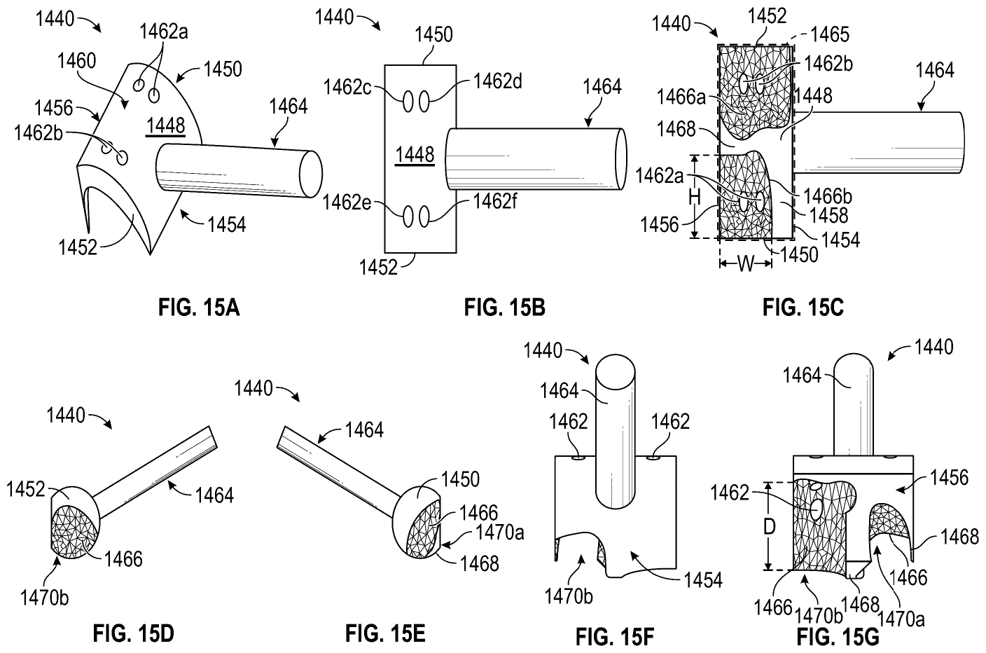

FIGS. 15A-15G are top perspective, top, bottom, anterior, posterior, medial, and lateral views respectively, of a positioning guide 1440 according to one embodiment. The positioning guide 1440 includes a body 1448, a proximal side 1450, a distal side 1452, a medial side 1454, a lateral side 1456, an inferior side 1458, a superior side 1460, at least one opening 1462, and, optionally, a handle 1464. Those of skill in the art will appreciate that the proximal side 1450 and distal side 1452 may be reversed from the example illustrated based on whether the positioning guide 1440 is for a left foot or a right foot.

The body 1448 provides the structural integrity for the positioning guide 1440. The body 1448 can be of a variety of shapes and sizes. The size, shape, and configuration of the positioning guide 1440 may be determined by the surgical procedure the positioning guide 1440 will be used with, by the preferences of a surgeon, by patient-specific features of a patient, a combination of these factors, or the like. For example, in a Lapidus procedure the body 1448 may be configured such that an optional handle 1464 extends medially to provide convenience as a surgeon uses the positioning guide 1440. In one embodiment, the body 1448 is made from a polymer and may be intended to be used for a particular patient. Furthermore, the body 1448 may be fabricated using additive or subtractive manufacturing techniques. Alternatively, or in addition, the body 1448 may be fabricated using molding methods.

In certain embodiments, the openings 1462 are sized, shaped, and configured to accept K-wire or pins or other fasteners 1446 or a drill bit. In certain embodiments, the positioning guide 1440 is patient-specific: fabricated, designed, and/or contoured for the needs of a specific patient and/or preferences of a surgeon. In one embodiment, the body 1448 includes a single proximal opening 1462a and a single distal opening 1462b. Alternatively, the body 1448 includes a plurality of proximal openings 1462a and a plurality of distal openings 1462b. The plurality of proximal openings 1462a and plurality of distal openings 1462a may be aligned vertically such that they are perpendicular to a long axis of a bone such as a metatarsal, when the metatarsal is in a predetermined position (e.g., a corrected or non-deformed position).

In one embodiment, when a metatarsal is in a deformed position (not predetermined position), the plurality of openings 1462 may not align with bones of a joint in order to provide reference features. In such a condition, a surgeon may reposition one or more of the bones of a joint until one or more of the bones are in the predetermined position, then the positioning guide 1440 can be used to provide reference features because the reference features will match model references used to design the positioning guide 1440.

Of course, in another embodiment, when a metatarsal is in a deformed position (not predetermined position), the plurality of openings 1462 may align with bones of a joint in order to provide reference features that can be used to guide a surgical procedure prior to repositioning one or more bones of a joint. In such an embodiment, the reference features may be used with instrumentation that facilitates resecting the bones and/or repositioning the resected bone(s) and/or reducing the bones and/or fixating the bones. Thus, the present disclosure supports an embodiment of a positioning guide 1440 that can be used after bones or a joint are moved to a predetermined position and/or an embodiment of a positioning guide 1440 that can be used before bones or a joint are moved to a predetermined position.

In one embodiment, the body 1448 may be as small as possible to serve its function and still be readily handled by a user. In certain embodiments, a body 1448 and/or handle 1464 can include one or more bevels that can facilitate handling and positioning of the positioning guide 1440 by a user. Referring to FIGS. 15D and 13E a lateral side 1456 of the body 1448 may be planar while a lateral side 1454 may be rounded. The planar side may facilitate use of the positioning guide 1440 without interference from other bones (e.g., a second metatarsal 210).

In another embodiment, the body 1448 may be transparent or at least transparent (e.g., radiolucent) to medical imaging. The positioning guide 1440 may include or be configured to accept one or more position indicators. At least one position indicator is positioned, configured, and/or arranged to indicate a position of the positioning guide 1440 in relation to a reference feature and/or anatomical structure. For example, the position indicator may indicate a position of the positioning guide 1440 relative to a joint (e.g., reference feature) of a patient.

In one embodiment, the positioning guide 1440 may include one or more openings 1462 that extend into the body 1448. The one or more openings 1462 may be configured to accommodate one or more fasteners 1446. In the illustrated embodiment, the one or more openings 1462 may be embodied as passages, holes, or openings, that extend from one side of the body 1448 to the other. The openings 1462 may serve as reference feature guides that may extend from a bone-facing side, such as an inferior side 1458, to a side opposite the bone-facing side, such as superior side 1460.

In certain embodiments, an osteotomy system includes the positioning guide 1440 and the positioning guide 1440 includes one or more features that can be used to provide, determine, deploy, install, configure, and/or establish at least one reference feature and/or anatomical references. The at least one reference feature can serve as an interface between an instrument used for a surgical procedure and a bone or a bone fragment of a patient. Those of skill in the art will appreciate that a reference feature may be implemented and/or embodied in a variety of different ways and/or with a variety of different apparatus, devices, structures, and/or systems, each of which is considered within the scope of the present disclosure.

In the illustrated embodiment, the at least one reference feature can be embodied as a hole or opening in one or more bones and/or one or more bone fragments. In another embodiment, the at least one reference feature can be embodied as a protrusion or other structure (e.g., a pin, post, bone screw, etc.) connected to or engaged with one or more bones and/or one or more bone fragments.

Advantageously, the positioning guide 1440 can be used to provide one or more different types of reference features. In one embodiment, the positioning guide 1440 includes one or more openings or holes that can serve for providing a reference feature (either or both holes in bone and/or posts or protrusions that extend from bone). The one or more openings or holes may extend from a superior side 1460 to an inferior side 1458 of the positioning guide 1440.

In the illustrated embodiment, the positioning guide 1440 includes a set of proximal openings 1462a and a set of distal openings 1462b. Those of skill in the art will appreciate that the positioning guide 1440 can include zero, one, or more proximal openings 1462a and zero, one, or more distal openings 1462b. Furthermore, those of skill in the art will appreciate that the proximal openings 1462a and/or distal openings 1462b can be positioned in a variety of locations within the body 1448. Advantageously, the number, size, position, and orientation of the proximal openings 1462a and/or distal openings 1462b can be patient-specific and/or can be determined or ordered by a surgeon based on the needs of the patient and/or surgeon preferences using the apparatuses, systems, and/or methods of the present disclosure.

The illustrated embodiment includes two proximal openings 1462a near a proximal end and two distal openings 1462b near a distal end. The two proximal openings 1462a can be adjacent to each other, can be spaced, can be aligned, or can have a variety of configurations that can assist a surgeon in performing a surgical procedure. The two distal openings 1462b can be adjacent to each other, can be spaced, can be aligned, or can have a variety of configurations that can assist a surgeon in performing a surgical procedure.

Advantageously, the proximal openings 1462a and/or the distal openings 1462b give the surgeon options for what kind of reference features the surgeon wants to use. In one embodiment, the surgeon may deploy pins or other fasteners through the proximal openings 1462a and/or distal openings 1462b and use the pins as reference features for one or more other steps of a surgical procedure. Alternatively, a surgeon may drill holes into one or more bones positioned adjacent to the positioning guide 1440 that align with the proximal openings 1462a and/or distal openings 1462b and use the holes in the one or more bones as reference features for one or more other steps of a surgical procedure. Where proximal openings 1462a and/or distal openings 1462b are used to form reference features, the holes formed using the proximal openings 1462a and/or distal openings 1462b can be referred to as anchor holes. Alternatively, or in addition, a surgeon may do a combination of deploying pins and/or drilling holes in one or more bones such that hole reference features and protrusion reference features are provided for one or more other steps of a surgical procedure. In one embodiment, a positioning guide 1440 can include a single proximal opening 1462a and two or more distal openings 1462b. In another embodiment, a positioning guide 1440 can include a single distal opening 1462a and two or more distal openings 1462b.

Referring now to FIGS. 15A and 15B, the positioning guide 1440 can include a first reference feature guide, such as proximal openings 1462a and a second reference feature guide, such as distal openings 1462b. In the illustrated embodiment, the first reference feature guide is a set of holes that extend from a non-bone-facing side of a (positioner) body 1448 (e.g., superior side 1460) to a bone-facing side of the positioner body 1448. Thus, the first reference feature guide and second reference feature guide may be implemented by a set of through holes.

In the illustrated embodiment, the first reference feature guide includes a first proximal opening 1462c and a second proximal opening 1462d positioned in the body 1448 such that pins or a drill bit in the openings will contact one of the bones of a joint (e.g., a medial cuneiform 202 of a TMT joint) when the positioning guide 1440 is used. Similarly, the second reference feature guide includes a first distal opening 1462e and a second distal opening 1462f positioned in the body 1448 such that pins or a drill bit in the openings will contact one of the bones of a joint (e.g., a first metatarsal 208 of a TMT joint) when the positioning guide 1440 is used. In one embodiment, at least one of the first proximal opening 1462c and the second proximal opening 1462d (the proximal set of holes) have a first trajectory that is configured to intersect a first bone of a joint (e.g., a medial cuneiform 202) at a perpendicular angle to the bone surface. Similarly, at least one of the first distal opening 1462e and second distal opening 1462f (the distal set of holes) has a second trajectory that is configured to intersect with a surface of a second bone of a joint (e.g., first metatarsal 208) at a perpendicular angle.

Having at least one of the first proximal opening 1462c and the second proximal opening 1462d intersect a bone surface of one bone at a perpendicular angle and having at least one of the first distal opening 1462e and the second distal opening 1462f intersect a bone surface of second bone at a perpendicular angle can be used in later stages of a surgical procedure to facilitate a favorable outcome. For example, a compressor/distractor 1438 can engage a reference feature (e.g., a pin, K-wire, bone hole, or the like) known to be perpendicular to a longitudinal axis of bones on opposite sides of a joint and compress the two bones together to accomplish a fusion of the bones.

However, those of skill in the art will appreciate that having each of the openings 1462 intersect bone surfaces at a perpendicular angle may result in a resection guide 1420 (cut guide) that engages with the reference features to move or slide away from the bones during an osteotomy. Therefore, in certain embodiments, at least one the proximal openings 1462a and/or at least one of the distal openings 1462b may extend through the body 1448 and intersect a bone surface of one or both of a first bone and a second bone at a trajectory at an oblique angle. In this manner, the opening 1462 at an oblique angle can be used to secure a reference feature like a pin deployed in a resection guide 1420 or a prong/spike extending a resection guide 1420 to prevent movement of the resection guide 1420 away from the bone(s) during an osteotomy.

A surgeon can use the positioning guide 1440 to establish or provide one or more reference features for use in one or more steps or stages of a surgical procedure. In one embodiment, the one or more reference features interface or provide an interface between one or more bones and/or bone fragments and an instrument that is configured to participate in addressing a bone condition of a patient. Said another way, the instrument is configured to engage with, couple to, register off of, or otherwise associate with or use the reference features as the instrument performs its function for a surgical procedure.

In one embodiment, an osteotomy system (e.g., osteotomy system 1400) according to the present disclosure may include a plurality of positioning guides 1440 (e.g., in a kit). Each positioning guide 1440 of the plurality may have a different set of configurations, positions, angles, and/or features that may be patient-specific and/or may be generic, and/or meet a surgeon's preferences. The plurality of positioning guides 1440 can be used intraoperatively by a surgeon for a surgical procedure. For example, a surgeon may use one or more bone models and/or models for instrumentation in formulating a preoperative plan, as described above. However, while the surgeon approves of the preoperative plan and instrumentation that is fabricated and provided for the surgical procedure, the surgeon may want to have one or more alternative options available during surgery. For example, a patient-specific positioning guide 1440 may be fabricated based on the preoperative planning that will change an IM angle from about ten degrees to about 7 degrees. However, the surgeon may also want the option of changing the IM angle to about 5 degrees or about 3 degrees or even 0 degrees (parallel with the second metatarsal 210). Accordingly, the surgeon may proscribe that two or three additional positioning guides 1440 be fabricated which will result in a change from an IM angle between the first metatarsal 208 and second metatarsal 210 from ten degrees to 7, 5, 3, or 0 degrees.

Since the positioning guide 1440 can be fabricated using additive manufacturing and can be made of relatively inexpensive materials such as a polymer, the surgeon can get the benefit of choosing which positioning guide 1440 to use during the surgical procedure. Thus, the surgeon may initially position the first metatarsal 208 using a first positioning guide 1440 and then review the change in the IM angle. If this is not satisfactory, the surgeon may position the first metatarsal 208 using a second positioning guide 1440 of the plurality of positioners (e.g., positioning guides 1440) and then review the change in the IM angle. With each of the plurality of positioning guides 1440 of an example system, the positioning guide 1440 engages a first bone (e.g., medial cuneiform 202) in its original position and engages a second bone (e.g., first metatarsal 208) in a predetermined position. Because each positioning guide 1440 of the plurality of positioning guide 1440 is designed for provided a different resulting IM angle, each predetermined position of the second bone is different for each of the positioning guides 1440. Once the surgeon identifies a satisfactory positioner/positioning guide 1440 from the plurality of positioner, this positioner may be used with its reference feature guides to provide reference features for subsequent stages of the surgical procedure.

FIG. 15A is a top perspective view of one embodiment of a positioning guide 1440. In the illustrated embodiment, the positioning guide 1440 includes a handle 1464. The handle 1464 may be an elongated structure that extends from the medial side 1454 away from the body 1448. In one embodiment, the handle 1464 is connected to the body 1448. In another embodiment, the handle 1464 and body 1448 may each include corresponding parts of a coupler (e.g., an internally threaded hole in the body and corresponding external threads on a distal end of the handle 1464) such that the handle 1464 can be coupled to the body 1448. The handle 1464 can have a round or circular cross section or a cross section in the shape of a geographic shape. In one embodiment, the handle 1464 may be a depression and/or curved indented portion of a surface of the body 1448 sized to accommodate a user's thumb or forefinger. Alternatively, the handle 1464 may include a set of depressions and/or curves of a surface of the body 1448 sized to accommodate a user's thumb and forefinger.

FIG. 15B is a top view of one embodiment of a positioning guide 1440.

FIG. 15C is a bottom view of one embodiment of a positioning guide 1440. In one embodiment, the positioning guide 1440 includes a bone engagement member 1465 on a bone-facing side of the body 1448. The bone engagement member 1465 serves to engage with one or more bones as the positioning guide 1440 is used. Advantageously, the bone engagement member 1465 is on a bone-facing side of the body 1448 such that the bone engagement member 1465 can readily engage with a bone.

In certain embodiments, the bone engagement member 1465 is configured to engage with a single bone. In another embodiment, the bone engagement member 1465 is configured to engage with two bones (e.g., a first bone and a second bone). In another embodiment, the bone engagement member 1465 is configured to engage with a plurality of bones. Furthermore, in certain embodiments, the bone engagement member 1465 engages with a single surface of one or more bones. Alternatively, or in addition, the bone engagement member 1465 engages with a plurality of surfaces of one or more bones. A bone engagement member 1465 can also be configured to engage with a landmark of the patient such as a depression or protrusion on a surface of a bone, a joint or space between bones or soft tissue of a patient. In the illustrated embodiment, the bone engagement member 1465 is configured to engage with a first bone and a second bone. In one embodiment, the bone engagement member 1465 is configured to engage with a medial cuneiform 202 of a patient and a first metatarsal 208 of a patient as part of a Lapidus surgical procedure.

In another embodiment, the bone engagement member 1465 is configured to engage with a first bone readily and subsequently to engage with a second bone that is moved into a predetermined position. In this manner, the bone engagement member 1465 may register with a first bone and use that registration to guide positioning of a second bone until the second bone is in a predetermined position for the second bone or the predetermined position defined for both the first bone and the second bone.

In certain embodiments, engagement of the bone engagement member 1465 with the first bone but not the second bone may be referred to as partial engagement. Once a second bone is moved (e.g., translated and/or rotated) into the predetermined position such that the bone engagement member 1465 engages the second bone and the bone engagement member 1465 remains engaged with the first bone, the bone engagement member 1465 is fully engaged with both the first bone and the second bone. In this condition, both the first bone and the second bone are in a predetermined position. Alternatively, each of the first bone and the second bone may have a predetermined position that is specific that that particular bone. In one embodiment, a predetermined position for a bone may be the same as its original position. When the bone engagement member 1465 is fully engaged with both the first bone and the second bone, a surgeon is assured that placement or formation of reference features using one or more reference feature guides will place and/or orient the one or more reference features in the same position as one or more model references that were used in a model of one or more bones of the patient prior to a surgical procedure.

Alternatively, or in addition, the bone engagement member 1465 may be configured to engage with both a first bone and second bone once both the first bone and the second bone are positioned or repositioned into a predetermined position for each bone.

As used herein, full engagement by a positioning guide 1440 refers to a condition in which the positioning guide 1440 engages at least both a first bone and second bone. The first bone and second bone are in or one or the other may have been moved to a predetermined position. Further, the predetermined position may be an original position for a bone. Partial engagement is a condition that is not full engagement by a positioning guide 1440 with both a first bone and second bone, regardless of whether either bone is moved from an original position to a predetermined position. If one or the other or both a first bone and a second bone are moved from an original position to a predetermined position and doing so results in the positioning guide 1440 engaging both the first bone and the second bone in a predetermined positioned, this condition is full engagement, but while one of the two bones is not yet in its predetermined position, the positioning guide 1440 is partially engaging one or the other or both of a first bone and second bone.

In one embodiment, the bone engagement member 1465 include a bone engagement surface 1466 configured to engage with a cortical surface of at least one of the first bone and the second bone. The bone engagement surface 1466 may include a contour that is at least partially determined based on a bone model of a patient's foot. As described above, the bone model may be defined based on medical imaging of the patient's foot.

In the illustrated embodiment, the bone engagement surface 1466 may be the whole inferior side 1458 of the body 1448. In the illustrated embodiment, the bone engagement surface 1466 is on the inferior side 1458. In another embodiment, the bone engagement surface 1466 may include two or more bone engagement surfaces, such as a first bone engagement surface 1466a and a second bone engagement surface 1466b. The first bone engagement surface 1466a is configured to engage with a first bone such as a medial cuneiform 202. The second bone engagement surface 1466b is configured to engage with a second bone such as a first metatarsal 208. In one embodiment, the first bone engagement surface 1466a may be configured to engage with only a medial cuneiform 202 of one foot of a patient and the second bone engagement surface 1466b may be configured to engage with only a first metatarsal 208 of the same foot of the patient.

In another embodiment, the bone engagement surface includes a three-dimensional surface. The three-dimensional surface may include an aspect that is patient-specific. Referring now to FIGS. 15C and 15G, one example of a bone engagement surface that is a three-dimensional surface is illustrated. The bone engagement surface 1466 may include a whole of the inferior side 1458, or one or both of the first bone engagement surface 1466a and second bone engagement surface 1466b, or a completely different structure.

Where a bone engagement surface is a three-dimensional surface, the bone engagement surface includes a height H, width W, and depth D. Each of these dimensions H, W, D may be patient-specific. In one embodiment, each of the dimensions H, W, D may be defined based on and/or using at least a portion of a bone model of a bone that the three-dimensional surface will receive. Of course, other attributes and/or features of the bone engagement surface may be patient-specific as well or instead of dimensions H, W, D. In one embodiment, the three-dimensional surface includes a variable H, W, and/or depth that is matched to change in a contour of a cortical surface of a bone that will be positioned within the three-dimensional surface. In another embodiment, one or more of the H, W, and/or depth of the three-dimensional surface is constant within the three-dimensional surface. In one such embodiment, the constant H, W, and/or depth of the three-dimensional surface may be greater than the distance needed such that a portion of the three-dimensional surface may not contact a portion of a surface of a bone.

In certain embodiments, the bone engagement member 1465 can be used to position the positioning guide 1440 in a desired position (e.g., predetermined position) against one or more bones and/or across one or more joints of a patient (a process referred to as registration, or registration to bone).

In certain embodiments, the positioning guide 1440 can include one or more landmark registration features 1468. In one embodiment, the landmark registration feature 1468 may extend from a bone-facing side (e.g., the inferior side 1458 in the illustrated embodiment). The landmark registration feature 1468 is configured to engage with a landmark of a patient. In one embodiment, the landmark may be a joint such as a tarsometatarsal (“TMT) joint of a foot of a patient. In one embodiment, the landmark registration feature 1468 is part of the bone engagement member 1465. In another embodiment, the landmark registration feature 1468 is a separate structure from the bone engagement member 1465. In one embodiment, the landmark registration features 1468 is defined based at least in part on a bone model or one or more bones of a patient. Thus, a landmark registration features 1468 may be patient-specific and/or may include one or more patient-specific aspects. In one embodiment, the landmark registration feature 1468 is configured to fit between articular surfaces of a first bone and a second bone of a joint. For example, a landmark registration features 1468 may be configured to fit within a TMT joint (e.g., medial cuneiform 202 and first metatarsal 208) of a foot of a patient.

Those of skill in the art will appreciate that a bone engagement member 1465 and/or landmark registration feature 1468 can be positioned on any surface or side of the positioning guide 1440.

FIG. 15D is an anterior view of the positioning guide 1440. FIG. 15E is a posterior view of the positioning guide 1440. Referring to FIGS. 15C-15G, note that the shape and size of the first bone engagement surface 1466a is different from the shape and size of the second bone engagement surface 1466b because the respective bones that each bone engagement surface 1466 is configured to engage is different. Advantageously, a surgeon can provide instructions on how deep the bone engagement surface 1466 (or three-dimensional surface) is for each bone. In this manner, a surgeon can request that the bone engagement surface 1466 extend further around one or more surfaces of a bone, when in use.

FIG. 15F is a medial view of the positioning guide 1440. In the illustrated embodiment, the medial side 1454 is a rounded surface. FIG. 15G is a lateral view of the positioning guide 1440. In the illustrated embodiment, the lateral side 1456 is a planar surface. The planar surface may facilitate positioning of the positioning guide 1440 without interference from other bones (e.g., second metatarsal 210), soft tissue, or the like.

FIGS. 15C-15G illustrate one example of one embodiment of a positioning guide 1440 that includes a bone engagement member 1465. In certain embodiments, the bone engagement member 1465 is one or more bone engagement surfaces 1466. Alternatively, or in addition, the bone engagement member 1465 may also be implemented as one or more openings 1470 that extends from the inferior side 1458 (e.g., bone-facing side) into the body 1448 but not completely through the body 1448. The one or more openings 1470 are configured to accept or receive one or more bones and/or portions of one or more bones. Furthermore, the one or more openings 1470 may be configured to accept or receive a single surface or a plurality of surfaces of one or more bones. Similar to a three-dimensional surface, the one or more openings 1470 may be defined based on a height H, width W, and depth D. In one embodiment, the one or more openings 1470 may be defined by a surface of the body 1448. The surface can be smooth, can include planar sections, and/or can include edges.

Alternatively, or in addition, the surface can be contoured to match (be a reverse mirror image) of a surface of a bone or a portion of a bone that the one or more openings 1470 will receive. In one embodiment, the size, shape, and/or contour of the one or more openings 1470 may be defined at least partially based on a bone model of one or more bones. As described herein, in certain embodiments, the bone model may be a bone model of bones of a patient and may be defined and/or derived from medical imaging of the patient. In one embodiment, the surface of the one or more openings 1470 is contoured to be patient-specific.

Alternatively, the surface of the one or more openings 1470 may be shaped (e.g., curved or have a depth) to engage the opening 1470 to accommodate one or more surfaces of a bone, but may not be shaped to match one or more surfaces of the one or more bones. In such an embodiment, the one or more openings 1470 can be said to be non-patient-specific. Alternatively, parts of one or more openings 1470 may be patient-specific while other parts are non-patient-specific.

Because the one or more openings 1470 are configured to accept or receive one or more bones, the one or more openings 1470 may also be referred to as bone engagement shaped openings. Those of skill in the art will appreciate that the one or more openings 1470 of a positioning guide 1440 may include two bones of a joint, but may also include openings configured to accept one or more bones adjacent to or around the joint or even other bones of the foot of a patient.

In the illustrated embodiment, the example positioning guide 1440 includes a first opening 1470a and a second opening 1470b. Each opening 1470a,b is configured to accept at least a portion of one of the first bone (e.g., a medial cuneiform 202) and the second bone (e.g., a first metatarsal 208). Alternatively, or in addition, each opening 1470a,b is determined at least partially based on a bone model of one or two bones of a joint. In the illustrated embodiment, the bone model includes a bone model of a medial cuneiform 202 and a first metatarsal 208. In certain embodiments, the bone models may be models specific for a particular set of patients (e.g., patient-matched). In another embodiment, the bone models may be models specific to a particular patient (e.g., patient-specific)

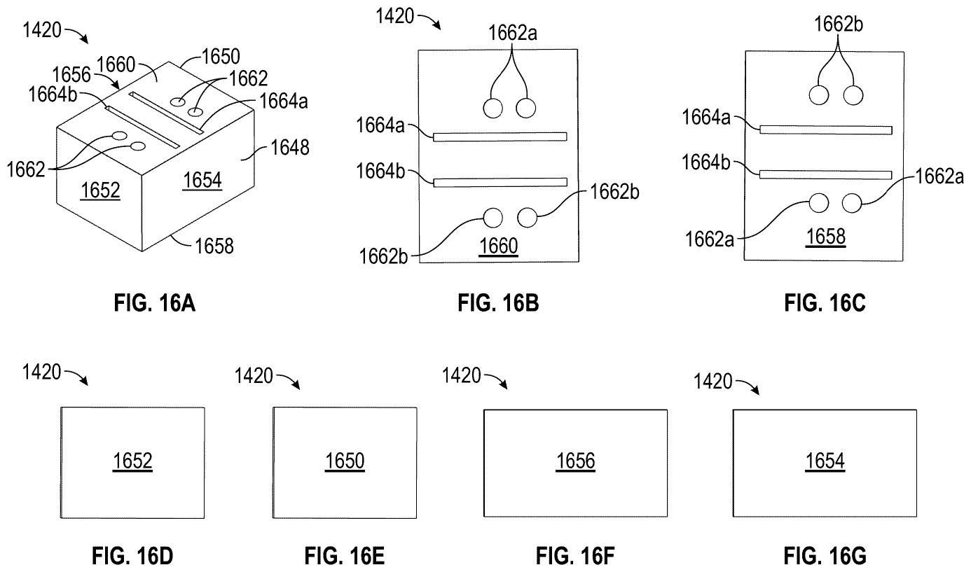

FIGS. 16A-16G are top perspective, top, bottom, anterior, posterior, medial, and lateral views respectively, of a resection guide 1420 according to one embodiment. The resection guide 1420 includes a body 1648, a proximal side 1650, a distal side 1652, a medial side 1654, a lateral side 1656, an inferior side 1658, a superior side 1660, at least one opening 1662, and, at least one resection feature 1664.

The body 1648 provides the structural integrity for the resection guide 1420. The body 1648 can be of a variety of shapes and sizes. The size, shape, and configuration of the resection guide 1420 may be determined by the surgical procedure the resection guide 1420 will be used with, by the preferences of a surgeon, by patient-specific features of a patient, a combination of these factors, or the like.

In the illustrated embodiment, the body 1648 is a rectangular cube shape. Such a shape may be advantageous because the shape is simple and easy to fabricate. The resection guide 1420 may be non-patient-specific.

In one embodiment, an osteotomy system according to the present disclosure may include a plurality of resection guides 1420 (e.g., in a kit). Each resection guide 1420 of the plurality may have a different set of configurations, positions, angles, and/or features that may be patient-specific and/or may be generic, and/or meet a surgeon's preferences. The plurality of resection guides 1420 can be used intraoperatively by a surgeon for a surgical procedure. In one embodiment, the resection guide 1420 may be one of a plurality in a kit that are patient-matched.

In certain embodiments, the resection guide 1420 is reusable and may be made of a durable material such as metal. Thus, following use the resection guide 1420 can be cleaned and prepared for re-use with another patient. In one osteotomy system 1400, the resection guide 1420 may be reusable and the positioning guide 1440 may be patient-specific and/or patient-matched. In one alternative embodiment, the resection guide 1420 may be patient-specific: fabricated, designed, and/or contoured for the needs of a specific patient and/or preferences of a surgeon.

In one embodiment, the resection guide 1420 may include one or more openings 1662 that extend into the body 1648. The one or more openings 1662 are configured to accommodate one or more fasteners 1646. In the illustrated embodiment, the one or more openings 1662 may be embodied as passages that extend from one side of the body 1648 to the other.

In certain embodiments, the openings 1662 are sized, shaped, and configured to engage with one or more reference features provided using a positioning guide 1440. For example, a surgeon may deploy pins or K-wires using a positioning guide 1440 and the opening 1662 are configured to accept the K-wires or pins or other fasteners 1646 or a drill bit.

In one embodiment, the body 1648 may be as small as possible to serve its function and still be readily handled by a user. In certain embodiments, a body 1648 and/or handle connected to the body 1648 can include one or more bevels that can facilitate handling and positioning of the resection guide 1420 by a user.

In another embodiment, the body 1648 may be transparent or at least transparent to medical imaging (e.g., radiolucent). Alternatively, or in addition, the body 1648 may include a marker that is opaque to medical imaging. The marker may be positioned in the body such that when the resection guide 1420 is in a predetermined position, the marker shows where cuts will be made in relation to other anatomical structures of the body. In one embodiment, the marker is made of a tantalum material or other radiopaque material that is visible in medical imaging. Alternatively, the marker is in the shape of a crosshair.

The resection guide 1420 may include or be configured to accept one or more position indicators. At least one position indicator is positioned, configured, and/or arranged to indicate a position of the resection guide 1420 in relation to a reference feature and/or anatomical structure. For example, the position indicator may indicate a position of the resection guide 1420 relative to a joint (e.g., reference feature) of a patient.

In certain embodiments, an osteotomy system includes the resection guide 1420 and the resection guide 1420 includes one or more features that can be used to provide, determine, deploy, install, configure, and/or establish at least one reference feature. The at least one reference feature can serve as an interface between an instrument used for a surgical procedure and a bone or a bone fragment of a patient. Those of skill in the art will appreciate that a reference feature may be implemented and/or embodied in a variety of different ways and/or with a variety of different apparatus, devices, structures, and/or systems, each of which is considered within the scope of the present disclosure.

In the illustrated embodiment, the at least one reference feature can be embodied as a hole or opening in one or more bones and/or one or more bone fragments. In another embodiment, the at least one reference feature can be embodied as a protrusion or other structure (e.g., a fastener, pin, post, bone screw, etc.) connected to, or engaged with, one or more bones and/or one or more bone fragments.

Advantageously, the resection guide 1420 can be used to provide one or more different types of reference features. In one embodiment, the resection guide 1420 includes one or more openings or holes that can serve for providing a reference feature (either or both holes in bone and/or posts or protrusions that extend from bone). The one or more openings or holes may extend from a superior side 1660 to an inferior side 1658 of the resection guide 1420.

In the illustrated embodiment, the resection guide 1420 includes a set of proximal openings 1662a and a set of distal openings 1662b. Those of skill in the art will appreciate that the resection guide 1420 can include zero, one, or more proximal openings 1662a and zero, one, or more distal openings 1662b. Furthermore, those of skill in the art will appreciate that the proximal openings 1662a and/or distal openings 1662b can be positioned in a variety of locations within the body 1648. Advantageously, the number, size, position, and orientation of the proximal openings 1662a and/or distal openings 1662b can be patient-specific and/or can be determined or ordered by a surgeon based on the needs of the patient and/or surgeon preferences using the apparatuses, systems, and/or methods of the present disclosure.

A surgeon can use the resection guide 1420 to establish or provide one or more reference features for use in one or more steps or stages of a surgical procedure. In one embodiment, the one or more reference features interface or provide an interface between one or more bones and/or bone fragments and an instrument that is configured to participate in addressing a bone condition of a patient. Said another way, the instrument is configured to engage with, couple to, register off of, or otherwise associate with or use the reference features as the instrument performs its function for a surgical procedure.

FIG. 16A is a top perspective view of one embodiment of a resection guide 1420. FIG. 16B is a top view of one embodiment of a resection guide 1420. FIG. 16C is a bottom view of one embodiment of a resection guide 1420. In one embodiment, the resection guide 1420 is not patient-specific and may be generic, patient-matched, or may be provided in a kit of a plurality of resection guides 1420, each configured differently. FIGS. 16B and 16C illustrate one example of resection features 1664. The resection features 1664 may extend through the body 1648 at a right angle to a superior side 1660 of the body 1648 and at a right angle to an inferior side 1658 of the body 1648. In addition, the embodiment of FIGS. 16B and 16C illustrate that the resection features 1664 are straight and parallel to each other. The straight resection features 1664 enable a surgeon to make a straight cut of bone positioned on the inferior side 1658 of the resection guide 1420. A straight cut is easier for a surgeon to complete than a non-straight cut (e.g., a curved cut).

In certain embodiments, the resection features 1664 are configured to form a straight cut and a cut that is perpendicular to an inferior side 1658 of the resection guide 1420 and perpendicular to superior side 1660 of the resection guide 1420. When a surgeon uses the resection guide 1420 with these resection feature 1664 on a joint such as a TMT joint, the osteotomy formed using a proximal resection feature 1664a will form a straight cut relative to a distal end of a first bone (e.g., a medial cuneiform 202) and the osteotomy formed using a distal resection feature 1664b will form a straight cut relative to a proximal end of a second bone (e.g., first metatarsal 208). This means that a cut face created using the proximal resection feature 1664a will be parallel to a cut face created using the distal resection feature 1664b. Because the cut faces are parallel when the two resected bones of a joint are fused the bones will maintain the same alignment in the sagittal plane 262. Such straight and perpendicular cuts are easier for a surgeon to complete. The present disclosure enables the use of straight cuts because the bones to be cut are positioned prior to performing the osteotomies. Specifically, one or both of the bones for the osteotomy are positioned when the positioning guide 1440 is used and reference features formed or created. The reference features are positioned where the bones will be once in a predetermined and/or corrected position. These same reference features can then be leveraged for use with a resection guide 1420 that provides creation of straight parallel and/or perpendicular cuts.

Those of skill in the art will appreciate that different resection guides 1420 can be used with the present system. Those other resection guides 1420 may include resection feature 1664 that are not completely parallel and/or may not be perpendicular to the bone(s). Such resection guides 1420 may be selected by a surgeon pre or intraoperatively to further adjust an orientation of one or more bones of a joint beyond what was initially planned in a preoperative plan. Further these adjustments can be made the sagittal plane 262, transverse plane 266, and/or the frontal plane 264.

In the illustrated embodiment, the resection guide 1420 does not include a bone engagement member 1465 such as a bone engagement surface 1666, one or more openings 1470, or three-dimensional surface. In other embodiments, the resection guide 1420 may include a bone engagement member 1465. Alternatively, or in addition, the resection guide 1420 may include one or more landmark registration features.

FIG. 16D is an anterior view of the resection guide 1420. FIG. 16E is a posterior view of the resection guide 1420. FIG. 16F is a lateral view of the resection guide 1420. FIG. 16G is a medial view of the resection guide 1420.

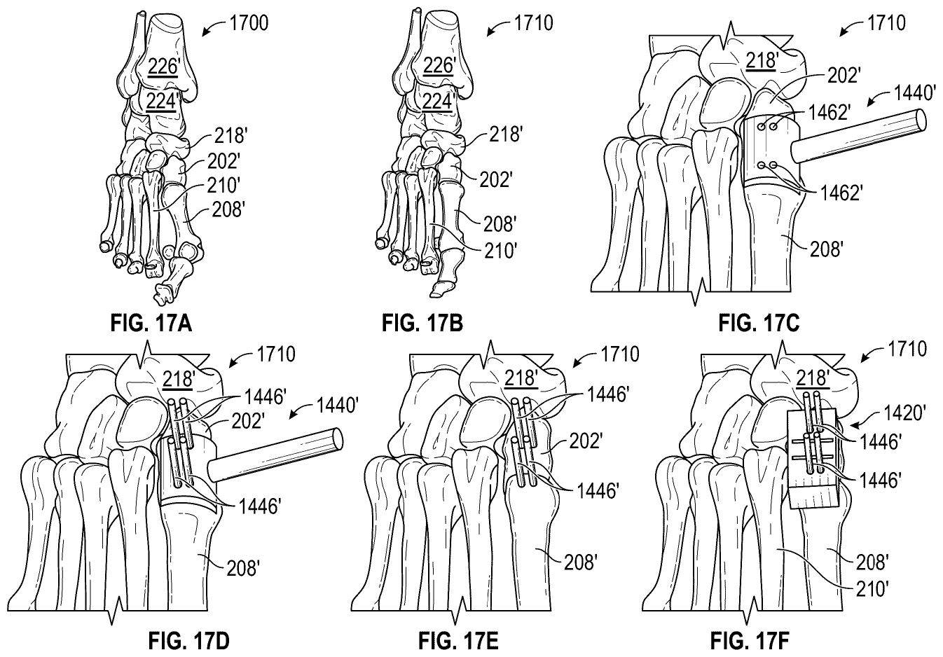

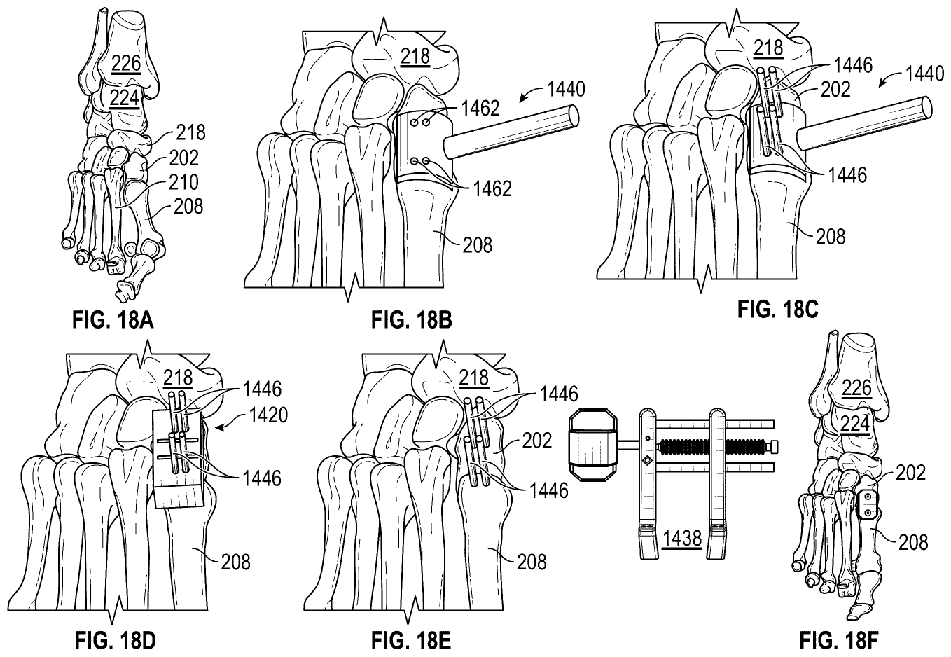

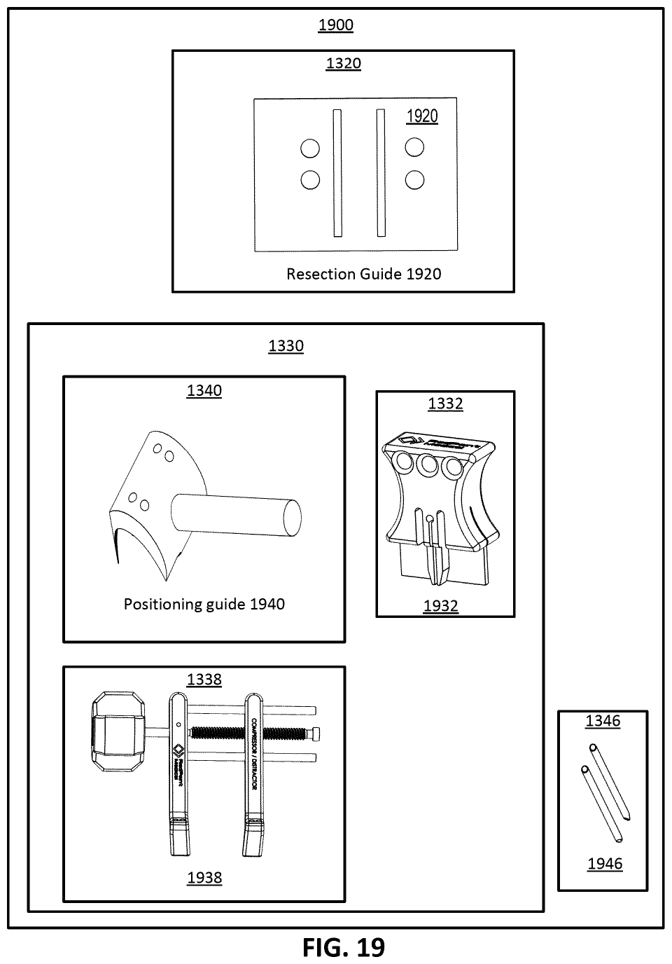

FIGS. 17A-17E illustrate different views of stages of providing instrumentation, according to one embodiment. FIG. 17A illustrates a model 1700 of patient anatomy. The model includes a plurality of bone models organized and configured to match the arrangement and configuration of the patient. Said another way, the model bones of the model 1700 are in an original position, a position that the patient first presented with when seeking medical assistance. Advantageously, as described above, the original position of the model bones can be derived from anatomic data 412 that can be provide and/or generated using medical imaging.

Where the present disclosure is used to correct a deformity, the orientation and position of model bones in the model 1700 may include the deformity. Thus, the model 1700 may also be referred to as a deformity model 1700. The model 1700 of the FIG. 17 includes a medial cuneiform 202′, a first metatarsal 208′, a second metatarsal 210′, a navicular 218′, a talus 224′, a tibia 226′, among other model bones. Note that the figures of FIG. 17 illustrate models representative of actual objects, tangible, physical objects, specifically bones, such as bones of a patient, and can include models of instrumentation to be used in a surgical procedure. The objects and models illustrated are identified with a reference number and a prime symbol to distinguish these objects from actual objects (e.g., bones and instrumentation) illustrated in FIG. 18 that include the same number without the prime symbol.

FIG. 17A Further illustrates that one or more bones of the foot have a bone condition, specifically the first metatarsal 208′ and bones of the big toe are deformed. This is indicated by the first metatarsal 208 that extends medially rather than parallel to the second metatarsal 210. The deformity may also include phalanges of the first metatarsal 208 that are oriented laterally rather than parallel to phalanges of a second metatarsal 210. In certain embodiments, model bones of the model 1700 that are deformed may be color coded to readily indicate that deformity exists. For example, in one embodiment the model bones in a deformed condition may be colored blue.

FIG. 17B illustrates the same model 1700 or models 1700 of FIG. 17A, except that a computing device (e.g., using a software program, a programmed hardware device, machine learning, artificial intelligence, or the like) and/or a user has revised the model 1700 to create a revised model 1710, also referred to as a corrected model and/or a revised CAD model. Producing a revised model 1710 may be based on the model 1700 or may be done using anatomic data 412 and/or other resources. In one embodiment, a computing device may reposition bone models in a model 1700 to create the revised model 1710. Alternatively, or in addition, a user (e.g., a surgeon) and/or a technician may reposition bone models using computer input devices either separately, in collaboration with each other, and/or in collaboration with a computing device to produce the revised model 1710. In certain embodiments, model bones of a revised model 1710 may be marked with an indication/indicator so the user can readily understand which model bones were repositioned. In one embodiment, the indicator is a surface coloring for the model bones that were repositioned to create the revised model 1710. For example, the surface coloring may be pink.

In the example embodiment of FIG. 17B, the first metatarsal 208′ and other bones of the big toe have been translated and/or rotated such that the first metatarsal 208′ is substantially parallel with the second metatarsal 210′. The revised model 1710 illustrates the bone model configuration that remediates a bone condition (e.g., a deformity such as a bunion). The revised model includes a model reference. In the illustrated example the model reference is the new position of the first metatarsal 208′. The model reference (predetermined or new position of one or more bones of the revised model 1710) can be used to design steps and/or instrumentation to correct a bone condition. At this stage, a user or a computing device can position a location for a hole or deployment of a pin into one or more of the first metatarsal 208′ and or the medial cuneiform 202′. Those holes and/or pins can serve as reference features for a surgical procedure (e.g., an osteotomy using a resection guide 1420).

FIG. 17B and revised model 1710 illustrate one meaning of a predetermined position. In FIG. 17B, each model bone in the revised model 1710 is shown in its predetermined position. This is a position that is determined before a surgical procedure is initiated or completed. For certain model bones, the original position is the same as the predetermined position. For other model bones, the model bone has been moved (e.g., translated and/or rotated) from an original position to the predetermined position.

Continuing now to FIG. 17C, FIG. 17C illustrates a close-up view of the bone model(s) of FIG. 17B. In FIG. 17C, a positioning guide 1440′ may be used to determine where to deploy one or more model fasteners 1446′ within model bones of the revised model 1710.

A computing device and/or a user separately and/or together may reference the medial cuneiform 202′, the first metatarsal 208′, and/or the second metatarsal 210′ (e.g., to measure an IM angle) to design and/or configure a model positioning guide 1440′.

FIG. 17C illustrates a model positioning guide 1440′ positioned over a TMT joint between a medial cuneiform 202′ and a first metatarsal 208′. The positioning guide 1440′ is configured to include model openings 1462 that will receive model fastener 1446′. In addition, the inferior side 1458 has been configured to include a bone engagement member 1465, such as a first bone engagement surface 1466a and a second bone engagement surface 1466b.

In one embodiment, the first bone engagement surface 1466a and second bone engagement surface 1466b may be formed by a Boolean subtraction operation between the body of the model positioning guide 1440′ and the model medial cuneiform 202′ and model first metatarsal 208′. It should be noted that the model positioning guide 1440′ is formed such that the bone engagement member 1465 fully engages both the medial cuneiform 202′ and the first metatarsal 208′. In this example, the medial cuneiform 202′ and first metatarsal 208′ are in a predetermined position. The medial cuneiform 202′ has not been moved from its original position to the predetermined position and the first metatarsal 208 has been moved from its original position to the predetermined position.

FIG. 17C illustrates that when a first metatarsal 208′ is not in the predetermined position and the positioning guide 1440′ fully engages with the medial cuneiform 202′, the positioning guide 1440′ will not fully engage with the first metatarsal 208′. Instead, the positioning guide 1440′ is partially engaged with the medial cuneiform 202′ and the first metatarsal 208′. However, with the positioning guide 1440′ fully engaged with the medial cuneiform 202′ and the first metatarsal 208′ moved to the predetermined position, the positioning guide 1440′ fully engages both the medial cuneiform 202′ and the first metatarsal 208′.

In one embodiment, with the positioning guide 1440′ in a desired position, with each of its openings 1462′ and a defined bone engagement member 1465, a user is ready to identify one or more model references for use in determining reference features during a surgical procedure. In the illustrated embodiment, the model references are model fasteners 1446′. The model fasteners 1446′ are deployed in the openings 1462′ and the next phase in the design/development process is illustrated in FIG. 17D.

Referring to FIG. 17D, a computing device and/or a user separately and/or together may reference and/or use the model fasteners 1446′ deployed using the positioning guide 1440′ to design and/or model a model resection guide 1420′. One or more proximal openings in the model positioning guide 1440′ and/or model resection guide 1420′ are configured to engage the model fasteners 1446′ of the medial cuneiform 202′. One or more distal openings in the model positioning guide 1440′ and/or model resection guide 1420′ are configured to engage the model fasteners 1446′ of the first metatarsal 208′.

FIG. 17D illustrates two model fasteners 1446′ deployed in the first metatarsal 208′ and two model fasteners 1446′ deployed in the medial cuneiform 202′. The model fasteners 1446′ may serve as model references that can be used to provide reference features.

FIG. 17E illustrates a next stage, in which the resection guide 1420′ is removed and the model fasteners 1446′ remain in the bones. FIGS. 17C-17E include the revised model 1710 showing the bones in the predetermined position. In the revised model 1710, at this stage the bones may remain in the predetermined position. Alternatively, or in addition, the model may include a feature that simulates deformed bones moving back towards an original position. In such an embodiment, the first metatarsal 208′ may rotate and/or reorient. However, this is not a concern because the fasteners 1446′ are positioned where needed for the desired correction.

FIG. 17F illustrates a stage in which a resection guide 1420's is determined. The resection guide 1420's is positioned with the openings 1662′ slid over the fasteners 1446′.

In certain embodiments, a user may use the resection guide 1420′ illustrated and modify the resection guide 1420's as needed, or a user may try differently configured resection guides 1420′ until the guide provides resection features (e.g., proximal resection feature 1664a′, distal resection feature 1664b′) that will produce the corrected configuration of the revised model 1710 illustrated in FIG. 17B.

At this stage, the modeled bones and/or modeled instruments can be fabricated or manufactured for use in a surgical procedure. In certain embodiments, the instruments are manufactured using additive manufacturing. Alternatively, or in addition, the instruments may be made from materials that are transparent to medical imaging.

FIGS. 18A-18F illustrate different views of stages of a surgical procedure, according to one embodiment. FIG. 18A illustrates the bones of a patient during a surgical procedure. In this example, the bones are in an original position and include a deformity. In certain embodiments, the original position is the position of bones of a patient when the patient presents for the surgical procedure. Alternatively, or in addition, the original position may be a position that bones of a patient are placed into in preparation for the surgical procedure. In one embodiment, the original position in an initial or starting position for bones that has been used for a bone model in preoperative planning for the surgical procedure.

FIG. 18B illustrates one stage during the surgical procedure. In this stage, a surgeon is deploying a positioning guide/positioner 1340, such as positioning guide 1440 (also referred to as a positioner 1440). In this stage, a surgeon seeks to reposition one or more bones of a patient from an original position to a predetermined position. In certain embodiments, this predetermined position is a position that remediates a condition of the patient. In one embodiment, the predetermined position may correct a deformity.

A surgeon can use a variety of techniques to reposition bones into the predetermined position. A few examples are provided here, however, those of skill in the art will appreciate that other techniques can be used that come within the scope of the present disclosure. Advantageously, the present disclosure provides a positioner 1440 to assist a surgeon in repositioning bones of the foot such that certain bones are translated and/or rotated into a predetermined position. In particular, the bone engagement member 1465 can facilitate and/or confirm/validate when one or more bones for a surgical procedure have been moved into a predetermined position for those bones.

In each of these examples, a surgeon may dissect soft tissue to gain access to cortical surfaces of one or more bones. Alternatively, or in addition, a surgeon may dissect and/or release certain soft tissue on or around a joint to enable repositioning of one or more bones. In the illustrated embodiment, a surgeon may dissect ligaments, tendons, periosteum, cartilage, and/or other connective tissue. Next, one or more of the following examples can be used by a surgeon.

In one example, a surgeon places a positioner 1440, such as a patient-specific positioner 1440, across a joint, such as a TMT joint so that a bone engagement member 1465 of the positioner 1440 engages with a first bone, such as a medial cuneiform 202. As illustrated, while the bone engagement member 1465 engages with the first bone, part of the bone engagement member 1465 or of the positioner 1440 may contact a second bone, such as a first metatarsal 208. This contact may be engagement with the second bone depending on how much deformity exists and/or whether the second bone has moved from its original position. More often, the bone engagement member 1465 will engage with the first bone and the bone engagement member 1465 will not engage with the second bone, until a user repositions the second bone.

Thus, in one example, a user may engage the first bone using the bone engagement member 1465 and then reposition the second bone until the second bone is in a predetermined position. With the first bone engaged with the bone engagement member 1465, once the second bone is repositioned into the predetermined position, the bone engagement member 1465 engages both the first bone and the second bone. In such a state/condition, the bone engagement member 1465 now fully engages with both the first bone and the second bone. When the positioner 1440, by way of the bone engagement member 1465 fully engages with both the first bone and the second bone, a user is assured that the bones are now in a position corresponding to a predetermined position planned in a preoperative plan using methods, systems, and/or apparatuses of the present disclosure.

In addition, when the bone engagement member 1465 engages with one of the first bone or the second bone which is in a predetermined position (which can include an original position) and does not engage (meaning the bone engagement member 1465 registers or seats or couples with the bone) with the second bone, this condition or state is a state of partial engagement between the bone engagement member 1465 and the first bone and the second bone. Often when the bone engagement member 1465 partially engages the first bone and the second bone, a user may further reposition one or more of the bones until full engagement is achieved between the bone engagement member 1465 and the first bone and the second bone. Alternatively, or in addition, a surgeon may determine that partial engagement between the bone engagement member 1465 and the first bone and the second bone is sufficient and may then proceed with the next stage of a surgical procedure.

Advantageously, partial engagement and full engagement of a bone engagement member 1465 with one of the bones is a condition that can be readily determined by a surgeon. In particular, when there is partial engagement a bone can be readily repositioned (minimal repositioning force applied) in relation to the bone engagement member 1465. In contrast, when there is full engagement a bone can not be readily repositioned (moderate to greater repositioning force required). With full engagement, the bone is said to be “locked” in engagement with the bone engagement member 1465.

In certain embodiments, a bone engagement member 1465 may be embodied as a first bone engagement surface and a second bone engagement surface. In such an embodiment, similar aspects of partial and full engagement can be determined based on whether bones engaged by the first bone engagement surface and a second bone engagement surface have been moved from an original position to a predetermined position. In one example, full engagement of a first bone and a second bone may include a first bone engagement surface engaging with the first bone and a second bone engagement surface engaging with the second bone where the first bone maintains its original position and the second bone is, or has been, repositioned from an original position to a predetermined position. In another example, partial engagement of a first bone and a second bone may include a first bone engagement surface engaging with the first bone and a second bone engagement surface engaging with the second bone where one or both bones are repositioned from an original position to a predetermined position.

As used herein, engagement between a bone engagement member 1465, a bone engagement surface 1466, a three-dimensional opening, and/or an opening refers to a relationship between the member and bone such that the member registers to the bone. Thus, where a device such as a positioner 1440 includes more than one engagement member, one engagement member may engage a bone while the other does not (partial engagement) or each engagement member may engage a bone (full engagement).

Referring now to FIG. 18B, a few examples of engagement between the positioner 1440 and bones of the joint are described. In this examples, the positioner 1440 includes a first bone engagement surface 1466a and a second bone engagement surface 1466b.

In one aspect, a surgeon may place the positioner 1440 on the medial cuneiform 202 such that the first bone engagement surface 1466a engages or registers with a dorsal surface (and/or a lateral surface and/or a medial surface) of the medial cuneiform 202. In this example, the medial cuneiform 202 maintains its original position. The second bone engagement surface 1466b does not engage with the first metatarsal 208. Thus, the positioner 1440 partially engages with the medial cuneiform 202 and the first metatarsal 208.

Next, a surgeon repositions the first metatarsal 208, while maintaining engagement between the first bone engagement surface 1466a and the medial cuneiform 202, until the second bone engagement surface 1466b engages with the first metatarsal 208. Now, the positioner 1440 fully engages with both the medial cuneiform 202 and the first metatarsal 208. Advantageously, the first metatarsal 208 is now in a predetermined position. The first metatarsal 208 is in the desired position for the surgical procedure. At this stage, the bones and the positioner 1440 are ready for deployment and/or formation of the reference features. A surgeon is assured that the bones are in substantially the same position are model bones used in preoperative planning.

In one variation to the example above, once a surgeon engages the first bone engagement surface 1466a with the medial cuneiform 202 a surgeon may deploy one or more fasteners 1446 through proximal openings 1462a which may secure the positioner 1440 to the medial cuneiform 202. The first metatarsal 208 may remain free to move in relation to the positioner 1440 until the second bone engagement surface 1466b engages the first metatarsal 208.

In another variation, a surgeon may review the position of the medial cuneiform 202 and the first metatarsal 208 once the positioner 1440 fully engages both bones and determine that a different amount of bone repositioning/correction is needed. Thus, a surgeon may exchange the positioner 1440 for another positioner that is configured to provide a different predetermined position for one or more bones (e.g., first metatarsal 208). In this manner, a surgeon can choose intraoperatively to use a different positioner 1440 in order to provide a desired outcome without be limited to a single positioner 1440.

In another aspect, a surgeon may place the positioner 1440 on the first metatarsal 208 such that the second bone engagement surface 1466b engages or registers with a dorsal surface (and/or a lateral surface and/or a medial surface) of the first metatarsal 208. In this example, the medial cuneiform 202 may maintains its original position and the first metatarsal 208 with the engaged positioner 1440 may be repositioned until the positioner 1440 engages the medial cuneiform 202. The first bone engagement surface 1466a may not yet engage with the medial cuneiform 202. Thus, the positioner 1440 partially engages with the medial cuneiform 202 and the first metatarsal 208.

Next, a surgeon repositions the first metatarsal 208, while maintaining engagement between the second bone engagement surface 1466b and the first metatarsal 208, until the first bone engagement surface 1466a engages with the medial cuneiform 202. Now, the positioner 1440 fully engages with both the medial cuneiform 202 and the first metatarsal 208. Advantageously, the first metatarsal 208 is now in a predetermined position. The first metatarsal 208 is in the desired position for the surgical procedure. At this stage, the bones and the positioner 1440 are ready for deployment and/or formation of the reference features. A surgeon is assured that the bones are in substantially the same position are model bones used in preoperative planning.

In one variation to the example above, once a surgeon engages the second bone engagement surface 1466b with the first metatarsal 208 a surgeon may deploy one or more fasteners 1446 through distal openings 1462b which may secure the positioner 1440 to the first metatarsal 208. The first metatarsal 208 may remain free to move in relation to the medial cuneiform 202 until the first bone engagement surface 1466a engages the medial cuneiform 202.

Advantageously, the engagement between the positioner 1440 and the two bones includes sufficient fidelity and congruence that engagement between a bone engagement member 1465 and a bone locks or snaps the bone into place, into engagement with the bone engagement member 1465. In one embodiment, the interface between the bone engagement member 1465 and a bone is such that a tactile feedback is generated when the bone moves from a disengaged state to an engaged state with the bone engagement member 1465. This tactile feedback can also be referred to as a palpable feedback. In the illustrated example of FIG. 18B, a surgeon moving the first metatarsal 208 can feel when the first metatarsal 208 moves into a predetermined position, a position planned preoperatively. When the first metatarsal 208 moves into the predetermined position, the bone engagement member 1465 (e.g., second bone engagement surface 1466b) engages (e.g., fully engages) with the first metatarsal 208. This palpable feedback provide confirmation to a surgeon that the first metatarsal 208 is in a desired position for the surgical procedure. Of course, engagement between a bone engagement member 1465 and a first bone (e.g., medial cuneiform 202) may also provide palpable feedback to the surgeon.

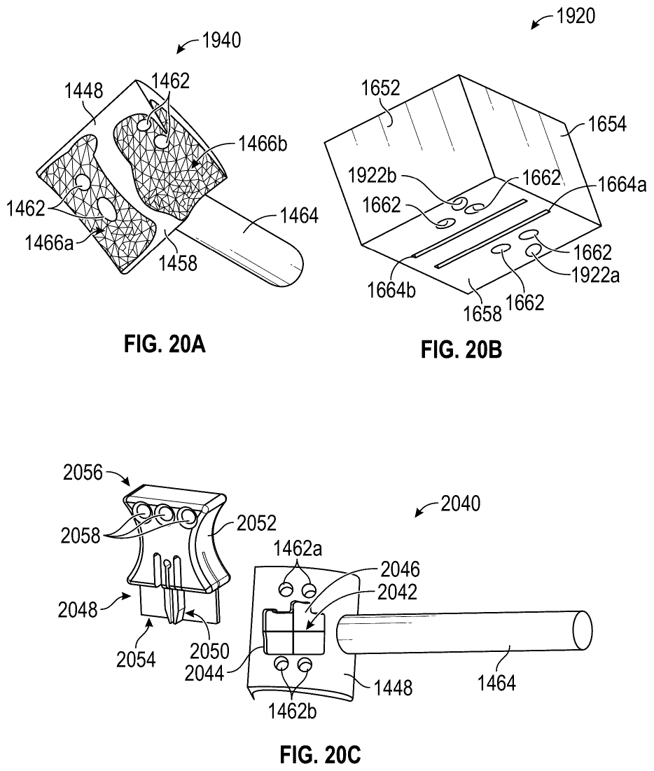

In certain embodiments, the positioner 1440 may also include an alignment guide (See FIGS. 19 and 20). In one example, an alignment guide may be implemented as a hole strategically placed within the handle of the positioner 1440 such that a K-wire that passes through the hole in the handle identifies the trajectory for the first metatarsal 208 once the surgical procedure is completed.

In certain embodiments, surgical procedures a surgeon may perform one or more osteotomies and then reposition bones of a patient to provide a desired outcome (e.g., deformity correction). Alternatively, a surgeon may reposition bones of a patient and subsequently perform one or more osteotomies to provide a desired outcome (e.g., deformity correction). In certain embodiments, for certain surgical procedures positioning bones before performing one or more osteotomies can simplify the type and kind of osteotomy needed to obtain a desired outcome. The present disclosure supports a surgeon in using either technique.

In addition, the present disclosure assists a surgeon in knowing, validating, and/or confirming that repositioned bones (either before or after an osteotomy) are in a desired (e.g., predetermined) position. Specifically, the present disclosure supports a surgeon in determining and confirming a desired position for bones in connection with an osteotomy through preoperative planning, use of anatomic data 412, intraoperative visualization (e.g., window, alignment guides, a positioner, and the like), surgical instruments and/or guides tailored to a particular surgical procedure and/or a particular patient, and tactile feedback (e.g., a surgeon can feel when a positioner engages both a first bone and second bone).

FIG. 18B illustrates one way to position bones of a patient for a surgical procedure. Specifically, a surgeon operates a positioner 1440 to place, deploy, and/or position the positioner 1440. The surgeon also may move and/or reposition one or more bones of a patient to achieve full engagement between the positioner 1440 and the bones. Of course, those of skill in the art will appreciate that use of a positioner 1440 manually by way of a handle is but one of a variety of ways to position patient bones as part of a surgical procedure.

For example, a jig may be associated and/or coupled to a patient such that the jig can position and/or orient one or more bones and/or one or more instruments for a surgical procedure. A jig may be secured to an ankle of a patient, to one or more metatarsals and/or one or more other midfoot bones of the patient. The jig may include one or more arms, struts, or that like that can be coupled to an instrument such as a positioner 1440. In such an embodiment, the positioner 1440 may have a coupler instead of or in addition to a handle 1464. A surgeon may operate one or more arms and/or struts and/or mechanisms of the jig to position the positioner 1440 in a desired position. Further, a surgeon may operate the jig to reposition one or more bones of a patient. In one example, the jig may be coupled to the first metatarsal 208 such that manipulating the jig moves and/or repositions the first metatarsal 208. Thus, a jig may be used by a surgeon to position a positioner 1440 and/or one or more bones that are used with the positioner 1440.

Referring to FIG. 18C, once the positioner 1440 is in the desired position and the bones engaged by the positioner 1440 are in a predetermined position, the openings 1462 (e.g., reference feature guides) of the positioner 1440 provide a guide for forming the reference features in the bone for the surgical procedure. FIG. 18C illustrates one example of forming the reference features in the bone. In one embodiment, the reference features are fasteners 1446 deployed one or more bones. In the illustrated embodiment, the bones are opposite bones of a joint such as a TMT joint. Referring now to FIG. 18C, at this stage a surgeon has the first metatarsal 208 in a predetermined position (e.g., desired position) that matches a corrected position of a bone model.

Next, the surgeon can use the openings 1462 (e.g., reference feature guides) to form one or more reference features in the bones. In this embodiment, the reference features are pins, such a fasteners 1446 or K-wires, deployed through the openings 1462 of the positioner 1440. With the fasteners 1446 deployed, the surgeon now has reference features that can be used for later stages of the surgical procedure. At this stage, the surgeon can remove the positioner 1440. When the positioner 1440 is removed, one or more bones (e.g., first metatarsal 208) may move from the predetermined position due to tension or stress of soft tissue, or gravity. However, this is not a concern because the fasteners 1446 remain in place as reference features for subsequent steps of the surgical procedure.

FIG. 18D illustrates a resection guide 1420 deployed over the fasteners 1446. Next, a surgeon can perform one or more osteotomies of one or more bones of the joint. FIG. 18E Illustrates a stage of the surgical procedure after resections have been made. The surgeon has removed the resection guide 1420. The fasteners 1446 may remain in place or may be removed. Remaining fasteners 1446 can be used for further subsequent steps of the surgical procedure. For example, the fasteners 1446 can be used by a compressor/distractor, such as compressor/distractor 1438. Arms of the compressor/distractor 1438 may slide over the fasteners 1446 and the compressor/distractor 1438 can be activated to either distract (to prepare cut faces for fusion) or compress (to prepare for fixation) the first metatarsal 208 and the medial cuneiform 202. In one embodiment, the compressor/distractor 1438 compresses a cut face of a first bone (e.g., medial cuneiform 202) against a cut face of a second bone (e.g., first metatarsal 208) by engaging a first reference feature (e.g., fastener 1446) of the first bone and a second reference feature (e.g., fastener 1446) of the second bone.

FIG. 18F illustrates the bones of the patient after the surgical procedure is completed. The cut face of the medial cuneiform 202 abuts the cut face of the first metatarsal 208. Fixation, in the form of a bone plate is secured to both the medial cuneiform 202 and the first metatarsal 208. Note that the first metatarsal 208 is in a corrected position relative to other bones of the foot.

FIG. 19 illustrates an exemplary osteotomy system 1900, according to one embodiment. The system 1900 may include similar components or modules to those described in relation to FIG. 14. The structures, features, and functions, operations, and configurations of the system 1900 may be similar or identical to components or modules of system 1400, like parts identified with similar reference numerals.

The osteotomy system 1900 may include a resection guide 1302, one example of which is resection guide 1920 (similar to resection guide 1420), one or more other complementary components 1330, such as positioning guide 1340, one example of which is positioner 1940, a compressor 1338, one example of which is compressor/distractor 1938, and/or an alignment guide 1332, one example of which is alignment guide 1932, and one or more fasteners 1346, such as fasteners 1946. The osteotomy system 1900 can be used for a variety of surgical procedures. In one embodiment, the resection guide 1920 is patient-matched. In one embodiment, the resection guide 1920 is not patient-specific. In one embodiment, the osteotomy system 1900 may include a plurality of resection guides 1920. One of the plurality of resection guide 1920 may be configured to make straight cuts perpendicular to a respective bone surface. Another of the plurality of resection guide 1920 may be configured to make straight cuts angled with respect to the bone surface. Another of the plurality of resection guide 1920 may be configured to make two straight cuts parallel to each other.

FIGS. 20A-20C illustrate different views of instrumentation of the system of FIG. 19, according to one embodiment. The positioner 1940 and resection guide 1920 may include similar components or modules to those described in relation to other embodiments. The structures, features, and functions, operations, and configurations may be similar or identical to components or modules of system 1400 or system 1900, like parts identified with similar reference numerals.

FIG. 20A illustrates an inferior perspective view of one embodiment of a positioner 1940. The positioner 1940 provides one or more reference features (e.g., fasteners deployed in openings 1462) for an osteotomy procedure. In certain embodiments, the reference features may comprise fasteners deployed in reference feature guides 1462 formed in a body of the positioner 1940. Each reference feature guide 1462 is configured to guide formation of a reference feature of a bone. Advantageously, these reference features correspond to one or more model references of a model.

The positioner 1940 includes a body 1448 having a bone-facing side (e.g., inferior side 1458) and a non-bone-facing side (e.g., superior side 1460). The positioner 1940 includes a first bone engagement surface 1466a, a second bone engagement surface 1466b, and a handle 1464. The first bone engagement surface 1466a is on the bone-facing side and is shaped to engage with a surface of a first bone. The surface of the first bone engagement surface 1466a is based at least in part on a model of a first bone. In one embodiment, the surface of the first bone is a surface of the bone. In another embodiment, the surface of the first bone is a surface of the bone that includes a covering of periosteum.

In one embodiment, the positioner 1940 is a patient-specific. The positioner 1940 may be made from a variety of materials. In one embodiment, the positioner 1940 is made from a material such as a polymer or a plastic, one example of which is Nylon 12.

The second bone engagement surface 1466b is on the bone-facing side and is shaped to engage with a surface of a second bone. The surface of the second bone engagement surface 1466b is based at least in part on a model of a second bone. In one embodiment, the surface of the second bone is a surface of the bone. In another embodiment, the surface of the second bone is a surface of the bone that includes a covering of periosteum. Like embodiments described above, in the illustrated embodiment, the first bone engagement surface 1466a is configured to engage a first bone (e.g., medial cuneiform 202) as the first bone maintains an original position and the second bone engagement surface 1466b does not engage a second bone first (e.g., metatarsal 208) until the second bone is repositioned to a predetermined position (e.g., a corrected position). The second bone engagement surface 1466b may contact the second bone, however the second bone engagement surface 1466b is configured to not engage and/or register with or to the second bone until the second bone has been moved into its predetermined position or into one of a plurality of predetermined positions. In one embodiment, a surgeon may define a plurality of predetermined positions for a bone in a preoperative plan.

The handle 1464 extends from the body 1448. In the illustrated embodiment, the handle 1464 extends from a medial side 1454 of the body 1448.

FIG. 20B illustrates an inferior perspective view of one embodiment of a resection guide 1920. The resection guide 1920 is configured to couple to one or more reference features (e.g., fasteners 1446 by way of openings 1662). The resection guide 1920 includes a resection body 1648 that includes a bone-facing side (e.g., inferior side 1658) and non-bone-facing side opposite the bone-facing side (e.g., superior side 1660). FIG. 20B illustrates that the inferior side 1658 is a planar surface in this embodiment. In other words, there is not a patient-specific surface and/or a bone engagement member on the inferior side 1658. In certain embodiments, this may be because the resection guide 1920 is not patient-specific. Instead, the resection guide 1920 may be patient-matched and/or may be reusable. The resection guide 1920 includes a first resection feature (e.g., proximal resection feature 1664a), a second resection feature (e.g., distal resection feature 1664b), and one or more bone attachment features 1922a,b.

The first resection feature 1664a extends through the resection body 1648 from the non-bone-facing side to the bone-facing side and is configured to form a first osteotomy in a first bone. The second resection feature 1664b extends through the resection body 1648 from the non-bone-facing side to the bone-facing side and is configured to form a second osteotomy in a second bone. In one embodiment, because the bones of patient are in a predetermined position, the proximal resection feature 1664a and distal resection feature 1664b may be less complicated resection cut guides than might otherwise be needed. In the illustrated embodiment, the proximal resection feature 1664a is configured to form a straight cut relative to a distal end of a first bone (e.g., a medial cuneiform 202) and the distal resection feature 1664b is configured to form a straight cut relative to a proximal end of a second bone (e.g., a first metatarsal 208).

The bone attachment feature 1922 serves to hold the resection guide 1920 in place while one or more osteotomies are performed. In one embodiment, the bone attachment feature 1922 is one or more holes or openings that extend from a bone-facing side (e.g., inferior side 1658) to a non-bone-facing side (e.g., superior side 1660) together with a fasteners 1446 that is deployed into bone through the hole/opening. The bone attachment feature 1922 is configured to secure the resection guide 1920 to one or more bones, such as a first bone and a second bone. In one embodiment, the openings and/or holes of the bone attachment feature 1922 intersect with a corresponding bone at an oblique angle. The intersection of the opening/hole of the bone attachment feature 1922 may be along a third trajectory. In this manner, even if fasteners 1446 in openings 1662 are perpendicular to the bones, the resection guide 1920 remains securely in place by way of fasteners 1446 in the openings of the bone attachment feature(s) 1922.

FIG. 20C illustrates an alternative embodiment of a positioner 2040. The positioner 2040 may include similar components or modules to those described in relation to other embodiments (e.g., resection guide 1920). The structures, features, and functions, operations, and configurations may be similar or identical to those components or modules, like parts identified with similar reference numerals. In the illustrated embodiment, the positioner 2040 is similar in many respects to those described herein.

In the illustrated embodiment, the positioner 2040 may differ from other embodiments because the positioner 2040 includes a position indicator 2042 and a window 2044. The position indicator 2042 indicates a position of a body of the positioner 2040 (e.g., body 1448) in relation to a first bone and a second bone. In certain embodiments, the position indicator 2042 may be configured to be positioned directly above and aligned with a joint of a patient (e.g., a TMT joint). In one embodiment, the position indicator 2042 includes a pair of pins or wires that are positioned in the body 1448 such that the pins or wires will align and/or be perpendicular to certain anatomical features of a patient such as a gap in a joint between a medial cuneiform 202 and a first metatarsal 208. In one embodiment, the position indicator 2042 is a crosshair that is positioned to mark a center of a joint and longitudinal axes for a joint.

Advantageously, a surgeon can place a positioner 2040 and then check the position indicator 2042 to confirm that the positioner 2040 is in a desired and/or predetermined position. Of course, other structures can be used and can serve as a position indicator 2042. For example, a straight line of a radiopaque material such as tantalum can be positioned in or on the body 1448 and serve as a position indicator 2042. In certain embodiments, a window 2044 can serve as a position indicator 2042.

In the illustrated embodiment, the window 2044 extends from a bone-facing side (e.g., inferior side 1458) to an opposite side of the body 1448. The window 2044 serves to enable a surgeon to visualize one or more bones on one side of the positioner 2040. In the illustrated embodiment, the window 2044 is positioned between the proximal openings 1462a and the distal openings 1462b.

The window 2044 can be made up of a single opening, a pair of openings, or a plurality of openings. In one embodiment, the window 2044 includes a plurality of openings that extend through the body. Of course, the window 2044 may have a variety of forms, shapes, sizes, and/or configurations. In one embodiment, the window 2044 may be a rectangular or other polygonal opening. The window 2044 may enable a surgeon to view past the positioner 2040 to visualize progress before, during or after one or more steps.

In one embodiment, the size and/or shape of the window 2044 can be determined at least in part by the surgeon which plans to use the positioner 2040. In certain embodiments, the window 2044 is sized and/or shaped to enable visualization of as much of the anatomy of the patient, while maintaining the structural integrity of the positioner 2040. Alternatively, or in addition, the window 906 can be a solid structure that is opaque to light but radiolucent to electromagnetic waves (e.g., X-rays, both still x-rays and fluoroscopy).

FIG. 20C also illustrates an alignment guide 1932 that a surgeon can use to check alignment between two or more bones of a patient. For example, as a surgeon positions the positioner 2040 a surgeon may want to check alignment between bones engaging the positioner 2040 and other bones of a patient. For example, in a Lapidus surgical procedure a surgeon may position a positioner 2040 on a medial cuneiform 202 and a first metatarsal 208 and then use an alignment guide 1932 to check and IM angle between the repositioned first metatarsal 208 and a second metatarsal 210.

The alignment guide 1932 is configured to couple to the positioner 2040 and to secure one or more position indicators (e.g., pins or K-wires). The position indicators coupled to the alignment guide 1932 identify a trajectory for one or more of a first bone and a second bone. The trajectory identified may be a trajectory after resecting one or more of the first bone and the second bone. Alternatively, the trajectory identified may be a trajectory before resecting one or more of the first bone and the second bone.

In the illustrated embodiment, the positioner 2040 includes one member 2046 of a coupler configured to engage a corresponding member of the coupler coupled to the alignment guide 1932. Those of skill in the art appreciate that various designs of a coupler may be used. In the illustrated embodiment, the coupler includes an opening such as member 2046 that may extend through the body 1448 and a post 2048. In one embodiment, the opening 2046 may include part of the window 2044. The post 2048 may include an engagement member 2050.

In one embodiment, the opening 2046 and the post 2048 engage each other in a friction fit. For example, the post 2048 may slide into the window 2044 and the engagement member 2050 may slide into the opening 2046. In one embodiment, the engagement member 2050 may include tabs that are biased outward and greater than a diameter of the opening 2046 such that the tabs engage the opening 2046 when inserted and release the opening when the tabs are pressed together.

The alignment guide 1932 includes a body 2052, an inferior end 2054, and superior end 2056 and one or more openings 2058 near the superior end 2056. The openings 2058 may be aligned. A surgeon may use the alignment guide 1932 by engaging the coupler to couple the alignment guide 1932 to the positioner 2040. Next, a surgeon may insert one or more K-wires through the openings 2058. The openings 2058 and alignment guide 1932 may be configured such that K-wires within the openings extend along an anterior-posterior axis and indicate the orientation and alignment of a first cuneiform and first metatarsal once an osteotomy procedure is completed. A surgeon may compare this alignment with the orientation and alignment of other bones of the patient (e.g., a second metatarsal). In this manner, a surgeon can confirm that an osteotomy procedure will accomplish the desired outcome once completed.

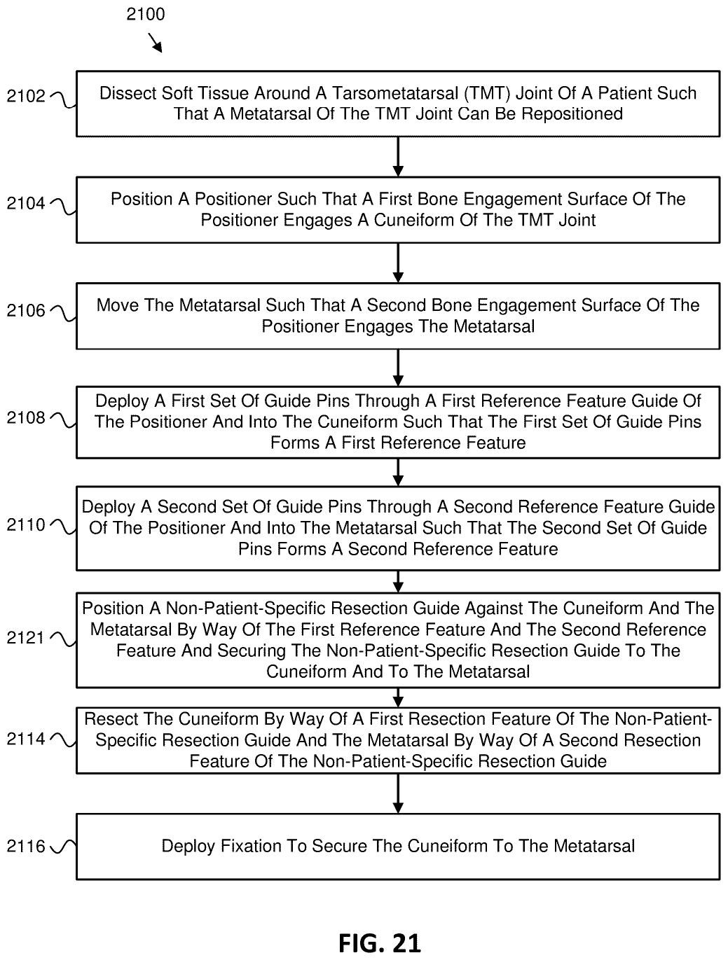

FIG. 21 is a flow chart of a method 2100, according to an example of the present disclosure. According to an example, one or more method blocks of FIG. 21 may be performed by a user, such as a surgeon.

As shown in FIG. 21, method 2100 may include dissecting soft tissue around a tarsometatarsal (TMT) joint of a patient such that a metatarsal of the TMT joint can be repositioned (block 2102).

As in addition shown in FIG. 21, method 2100 may include positioning a positioner such that a first bone engagement surface of the positioner engages a cuneiform of the TMT joint (block 2104). For example, device may position a positioner such that a first bone engagement surface of the positioner engages a cuneiform of the TMT joint, as described above.

As also shown in FIG. 21, method 2100 may include moving the metatarsal such that a second bone engagement surface of the positioner engages the metatarsal (block 2106). For example, a surgeon may move the metatarsal such that a second bone engagement surface of the positioner engages the metatarsal, as described above. In one embodiment, the surgeon may move the metatarsal such that a second bone engagement surface of the positioner fully engages the metatarsal. Alternatively, or in addition, in one embodiment, a surgeon may move the first metatarsal 208 until the surgeon feels the first metatarsal 208 engage fully with a second bone engagement surface of the positioner. This the moving step, block 2106, may include feeling (e.g., by way of palpable feedback) for the first metatarsal 208 engage with the second bone engagement surface. Alternatively, the method 2100 may include an additional step of feeling or checking whether the first metatarsal 208 is fully engaged with the second bone engagement surface of the positioner and/or checking whether the medial cuneiform 202 is fully engaged with the first bone engagement surface of the positioner.

As further shown in FIG. 21, method 2100 may include deploying a first set of guide pins through a first reference feature guide of the positioner and into the cuneiform such that the first set of guide pins forms a first reference feature (block 2108). For example, a surgeon may deploy a first set of guide pins through a first reference feature guide of the positioner and into the cuneiform such that the first set of guide pins forms a first reference feature, as described above. As in addition shown in FIG. 21, method 2100 may include deploying a second set of guide pins through a second reference feature guide of the positioner and into the metatarsal such that the second set of guide pins forms a second reference feature (block 2110). For example, a surgeon may deploy a second set of guide pins through a second reference feature guide of the positioner and into the metatarsal such that the second set of guide pins forms a second reference feature, as described above.

As also shown in FIG. 21, method 2100 may include positioning a non-patient-specific resection guide against the cuneiform and the metatarsal by way of the first reference feature and the second reference feature and securing the non-patient-specific resection guide to the cuneiform and to the metatarsal (block 2112). For example, a surgeon may position a non-patient-specific resection guide against the cuneiform and the metatarsal by way of the first reference feature and the second reference feature and securing the non-patient-specific resection guide to the cuneiform and to the metatarsal, as described above.

As further shown in FIG. 21, method 2100 may include resecting the cuneiform by way of a first resection feature of the non-patient-specific resection guide and the metatarsal by way of a second resection feature of the non-patient-specific resection guide (block 2114). For example, a surgeon may resect the cuneiform by way of a first resection feature of the non-patient-specific resection guide and the metatarsal by way of a second resection feature of the non-patient-specific resection guide, as described above.

As in addition shown in FIG. 21, method 2100 may include deploying fixation to secure the cuneiform to the metatarsal (block 2116). For example, a surgeon may deploy fixation to secure the cuneiform to the metatarsal, as described above.

It should be noted that while FIG. 21 shows example blocks of method 2100, in some implementations, method 2100 may include additional blocks, fewer blocks, different blocks, or differently arranged blocks than those depicted in FIG. 21. Additionally, or alternatively, two or more of the blocks of method 2100 may be performed in parallel.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Alternatively, or in addition, any of the systems, devices, and/or apparatuses herein can be implemented using fewer than the elements and/or components described in the presented embodiments. In addition, components and/or elements and/or structures in one embodiment may be used in other embodiments to replace a component or structure and/or to augment the embodiment within the scope of the claims and present disclosure.

Reference throughout this specification to “an embodiment” or “the embodiment” means that a particular feature, structure or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, Figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. Elements recited in means-plus-function format are intended to be construed in accordance with 35 U.S.C. § 112 Para. 6. It will be apparent to those having skill in the art that changes may be made to the details of the above-described embodiments without departing from the underlying principles set forth herein.

While specific embodiments and applications of the present disclosure have been illustrated and described, it is to be understood that the scope of this disclosure is not limited to the precise configuration and components disclosed herein. Various modifications, changes, and variations which will be apparent to those skilled in the art may be made in the arrangement, operation, and details of the methods and systems of the present disclosure set forth herein without departing from it spirit and scope.

What is claimed is:

1. A patient-specific apparatus for facilitating an osteotomy procedure, the apparatus comprising:
a body;
a patient-specific bone engagement member on a bone-facing side of the body, the patient-specific bone engagement member configured to engage a first bone and a second bone to correct a bone condition of the second bone relative to the first bone; and
a reference feature guide configured with the body to guide formation of a reference feature for at least one of the first bone and the second bone.

2. The apparatus of claim 1, wherein the patient-specific bone engagement member is configured to fully engage with both the first bone and the second bone when both bones are in a predetermined position.

3. The apparatus of claim 1, wherein the patient-specific bone engagement member is configured to partially engage with one of the first bone and the second bone when one of the first bone and the second bone is in a predetermined position.

4. The apparatus of claim 1, wherein the patient-specific bone engagement member comprises a bone engagement surface configured to engage with a cortical surface of at least one of the first bone and the second bone, the bone engagement surface having a contour at least partially determined based on a bone model of a patient's foot.

5. The apparatus of claim 4, wherein the bone engagement surface comprises a first bone engagement surface configured to engage with the first bone and a second bone engagement surface configured to engage with the second bone.

6. The apparatus of claim 5, wherein the first bone engagement surface is configured to engage with the first bone and the second bone engagement surface is configured to engage with the second bone when at least one of the first bone and the second bone is repositioned from an original position to a predetermined position.

7. The apparatus of claim 5, wherein the first bone engagement surface is configured to engage with the first bone and the second bone engagement surface is configured to engage with the second bone when the first bone maintains an original position and the second bone is repositioned from an original position to a predetermined position.

8. The apparatus of claim 5, wherein the second bone engagement surface is configured to provide palpable feedback to a user when the second bone moves to a predetermined position.

9. The apparatus of claim 4, wherein the bone engagement surface comprises a three-dimensional surface having an aspect that is patient-specific.

10. The apparatus of claim 1, wherein the patient-specific bone engagement member comprises an opening in the body, the opening configured to accept at least a portion of one of the first bone and the second bone and at least partially determined based on a bone model.

11. The apparatus of claim 1, wherein the reference feature guide comprises at least one opening in the body that extends from a bone-facing side to a side opposite the bone-facing side.

12. The apparatus of claim 1, further comprising:
a handle configured with the body to extend the handle away from the body; and
a landmark registration feature that extends from a bone-facing side of the body, the landmark registration feature configured to engage with a landmark of a patient.

13. The apparatus of claim 1, further comprising:
a position indicator that indicates a position of the body in relation to the first bone and the second bone; and
a window extending from a bone-facing side of the body to an opposite side of the body.

14. The apparatus of claim 1, wherein the first bone is a cuneiform and the second bone is a metatarsal.

15. A system for an osteotomy procedure comprising:
a positioner that provides a reference feature for an osteotomy procedure, the reference feature corresponding to a model reference of a model, the positioner comprising:
a positioner body having a bone-facing side and a non-bone-facing side;
a first bone engagement surface of the bone-facing side, the first bone engagement surface shaped to engage a surface of a first bone of a patient, the surface shaped based at least in part on a model of the first bone;
a second bone engagement surface of the bone-facing side, the second bone engagement surface shaped to engage a surface of a second bone of a patient, the surface shaped based at least in part on a model of the second bone;
a first reference feature guide formed in the positioner body and configured to guide formation of a first reference feature for the first bone;
a second reference feature guide formed in the positioner body and configured to guide formation of a second reference feature for the second bone;
a handle coupled to the positioner body; and
a resection guide that couples to the reference feature, the resection guide comprising:
a resection body having a bone-facing side and a non-bone-facing side;
a first resection feature extending through the resection body from the non-bone-facing side of the resection body to the bone-facing side, the first resection feature configured to guide a cutting tool to form a first osteotomy in the first bone;
a second resection feature extending through the resection body from the non-bone-facing side of the resection body to the bone-facing side, the second resection feature configured to guide a cutting tool to form a second osteotomy in the second bone; and
a bone attachment feature configured to secure the resection guide to at least one of the first bone and the second bone.

16. The system of claim 15, wherein the first bone engagement surface is configured to engage the first bone when the first bone maintains an original position and the second bone engagement surface does not engage the second bone until the second bone is repositioned to a predetermined position.

17. The system of claim 15, further comprising:
a compressor configured to compress a cut face of the first bone against a cut face of the second bone by engaging the first reference feature of the first bone and the second reference feature of the second bone; and
an alignment guide configured to couple to the positioner and to secure a position indicator, the position indicator identifying a trajectory for one of the first bone and second bone after resecting the first bone and the second bone.

18. The system of claim 15, wherein the first reference feature guide and second reference feature guide comprise a set of holes that extend from the non-bone-facing side to the bone-facing side of the positioner body and wherein at least a first hole of the set of holes has a first trajectory that intersects a surface of the first bone at a perpendicular angle and at least a second hole of the set of holes has a second trajectory that intersects a surface of the second bone at a perpendicular angle.

19. The system of claim 18, wherein the bone attachment feature comprises a hole that extends from the non-bone-facing side to the bone-facing side of the resection body and has a third trajectory that intersects a surface of at least one of the first bone and the second bone at an oblique angle.

20. The system of claim 15, wherein:
the positioner is a patient-specific instrument made from a polymer;
the first resection feature is configured to form a straight cut relative to a distal end of the first bone;
the second resection feature is configured to form a straight cut relative to a proximal end of the second bone; and
wherein the bone-facing side of the resection guide is planar.

21. The system of claim 15, comprising a plurality of positioners each configured to engage the first bone in an original position and engage the second bone in a different predetermined position.

22. A method for remediating a bone condition present in a patient's foot, the method comprising:
dissecting soft tissue around a tarsometatarsal (“TMT”) joint of a patient such that a metatarsal of the TMT joint can be repositioned;
positioning a positioner such that a first bone engagement surface of the positioner engages a cuneiform of the TMT joint;
moving the metatarsal such that a second bone engagement surface of the positioner engages the metatarsal;
deploying a first set of guide pins through a first reference feature guide of the positioner and into the cuneiform such that the first set of guide pins forms a first reference feature;
deploying a second set of guide pins through a second reference feature guide of the positioner and into the metatarsal such that the second set of guide pins forms a second reference feature;
positioning a non-patient-specific resection guide against the cuneiform and the metatarsal by way of the first reference feature and the second reference feature and securing the non-patient-specific resection guide to the cuneiform and to the metatarsal;
resecting the cuneiform by way of a first resection feature of the non-patient-specific resection guide and the metatarsal by way of a second resection feature of the non-patient-specific resection guide; and
deploying fixation to secure the cuneiform to the metatarsal.
